(19) United States
(12) Patent Application Publication
(10) Pub. No.: US 2005/0148029 A1
(43) Pub. Date: Jul. 7, 2005

(54) Methods and compositions for determining treatment regimens in systemic inflammatory response syndromes

(75) Inventors: Kenneth F. Buechler, Rancho Santa Fe, CA (US); Joseph Michael Anderberg, Encinitas, CA (US); Paul H. McPherson, Encinitas, CA (US)

Correspondence Address: FOLEY & LARDNER, P.O. BOX 80278, SAN DIEGO, CA, 92138-0278, US

(73) Assignee: Biosite, Inc.

(21) Appl. No.: 11/022,552

(22) Filed: Dec. 23, 2004

Related U.S. Application Data
(63) Continuation-in-part of application No. 10/952,275, filed on Sep. 27, 2004.
(60) Provisional application No. 60/507,113, filed on Sep. 29, 2003.
(60) Provisional application No. 60/532,777, filed on Dec. 23, 2003.
(60) Provisional application No. 60/558,945, filed on Apr. 2, 2004.

(51) Int. Cl. G01N 33/53; G01N 33/00

(52) U.S. Cl. 435/7.1; 436/86

(57) ABSTRACT

The present invention relates to methods and compositions for symptom-based differential diagnosis, prognosis, and determination of treatment regimens in subjects. In particular, the invention relates to methods and compositions selected to rule in or out SIRS, or for differentiating sepsis, severe sepsis, septic shock and/or MODS from each other and/or from non-infectious SIRS.

The present application is a continuation in part of U.S. patent application Ser. No. 10/952,275 filed Sep. 27, 2004, entitled METHODS AND COMPOSITIONS FOR THE DIAGNOSIS OF SEPSIS (pending), which claims priority to U.S. Provisional Applications 60/507,113 filed Sep. 29, 2003, 60/532,777 filed Dec. 23, 2003, and 60/558,945 filed Apr. 2, 2004, each of which is hereby incorporated in its entirety and from each of which priority is hereby claimed.

### FIELD OF THE INVENTION

The present invention relates to the identification and use of diagnostic markers related to sepsis. In a various aspects, the invention relates to methods and compositions for use in assigning a treatment pathway to subjects suffering from SIRS, sepsis, severe sepsis, septic shock and/or multiple organ dysfunction syndrome.

### BACKGROUND OF THE INVENTION

The following discussion of the background of the invention is merely provided to aid the reader in understanding the invention and is not admitted to describe or constitute prior art to the present invention.

The term “sepsis” has been used to describe a variety of clinical conditions related to systemic manifestations of inflammation accompanied by an infection. Because of clinical similarities to inflammatory responses secondary to non-infectious etiologies, identifying sepsis has been a particularly challenging diagnostic problem. Recently, the American College of Chest Physicians and the American Society of Critical Care Medicine (Bone et al., Chest 101: 1644-53, 1992) published definitions for “Systemic Inflammatory Response Syndrome” (or “SIRS”), which refers generally to a severe systemic,response to an infectious or non-infectious insult, and for the related syndromes “sepsis,” “severe sepsis,” and “septic shock,” and extending to multiple organ dysfunction syndrome (“MODS”). These definitions, described below, are intended for each of these phrases for the purposes of the present application.

“SIRS” refers to a condition that exhibits two or more of the following:
a temperature >38° C. or <36° C.;
a heart rate of >90 beats per minute (tachycardia);
a respiratory rate of >20 breaths per minute (tachypnea) or a PₐCO₂<4.3 kPa; and
a white blood cell count >12,000 per mm³, <4,000 per mm³, or >10% immature (band) forms.

“Sepsis” refers to SIRS, further accompanied by a clinically evident or microbiologically confirmed infection. This infection may be bacterial, fungal, parasitic, or viral.

“Severe sepsis” refers to sepsis, further accompanied by organ hypoperfusion made evident by at least one sign of organ dysfunction such as hypoxemia, oliguria, metabolic acidosis, or altered cerebral function.

“Septic shock” refers to severe sepsis, further accompanied by hypotension, made evident by a systolic blood pressure <90 mm Hg, or the requirement for pharmaceutical intervention to maintain blood pressure.

MODS (multiple organ dysfunction syndrome) is the presence of altered organ function in a patient who is acutely ill such that homeostasis cannot be maintained without intervention. Primary MODS is the direct result of a well-defined insult in which organ dysfunction occurs early and can be directly attributable to the insult itself. Secondary MODS develops as a consequence of a host response and is identified within the context of SIRS.

A systemic inflammatory response leading to a diagnosis of SIRS may be related to both infection and to numerous non-infective etiologies, including bums, pancreatitis, trauma, heat stroke, and neoplasia. While conceptually it may be relatively simple to distinguish between sepsis and non-septic SIRS, no diagnostic tools have been described to unambiguously distinguish these related conditions. See, e.g., Llewelyn and Cohen, Int. Care Med. 27: S10-S32, 2001. For example, because more than 90% of sepsis cases involve bacterial infection, the “gold standard” for confirming infection has been microbial growth from blood, urine, pleural fluid, cerebrospinal fluid, peritoneal fluid, synnovial fluid, sputum, or other tissue specimens. Such culture has been reported, however, to fail to confirm 50% or more of patients exhibiting strong clinical evidence of sepsis. See, e.g., Jaimes et al., Int. Care Med 29: 1368-71, published electronically Jun. 26, 2003.

The physiologic responses leading to the systemic manifestations of inflammation in sepsis remain unclear. Activation of immune cells occurs in response to the LPS endotoxin of gram negative bacteria and exotoxins of gram positive bacteria. This activation leads to a cascade of events mediated by proinflammatory cytokines, adhesion molecules, vasoactive mediators, and reactive oxygen species. Various organs, including the liver, lungs, heart, and kidney are affected directly or indirectly by this cascade. Sepsis is also associated with disseminated intravascular coagulation (“DIC”), mediated presumably by cytokine activation of coagulation. Fluid and electrolyte balance are also affected by increases in capillary perfusion and reduced oxygenation of tissues. Unchecked, the uncontrolled inflammatory response created can lead to ischemia, loss of organ function, and death.

Despite the availability of antibiotics and supportive therapy, sepsis represents a significant cause of morbidity and mortality. A recent study estimated that 751,000 cases of severe sepsis occur in the United States annually, with a mortality rate of from 30-50%. Angus et al., Crit. Care Med. 29: 1303-10, 2001. Recently, an organization of medical care groups referred to as the “Surviving Sepsis Campaign” issued guidelines for managing subjects suffering from severe sepsis and septic shock. Dellinger et al., Crit. Care Med. 32: 858-873, 2004. These guidelines draw from, amongst other sources, the “Early Goal Directed Therapy” therapy regimen developed by Rivers and colleagues. See, e.g., New Engl. J. Med. 345: 1368-77. 2001.

Several laboratory tests have been investigated or proposed for use, in conjunction with a complete clinical examination of a subject, for the diagnosis and prognosis of sepsis. See, e.g., U.S. Pat. Nos. 5,639,617 and 6,303,321; and Charpentier et al., Crit. Care Med. 32: 660-65, 2004; Castillo et al., Int. J. Infect. Dis. 8: 271-74, 2004; Chua and Kang-Hoe, Crit. Care 8: R248-R250, 2004; Witthaut et al., Int. Care Med. 29: 1696-1702, 2003; Jones and Kline, Ann. Int. Med. 42: 714-15, 2003; Maeder et al., Swiss Med. Wkly. 133: 515-18, 2003; Giamarellos-Bourboulis et al., Intensive Care Med. 28: 1351-56, 2002; Harbarth et al., Am. J. Respir. Crit. Care Med. 164: 396-402, 2001; Martin et al., Pediatrics 108: URL: http://www.pediatrics.org/cgi/content/full/108/4/e61, 2001; and Bossink et al., Chest 113: 1533-41, 1998.

### BRIEF SUMMARY OF THE INVENTION

The present invention relates to the identification and use of markers for the detection of sepsis, the differentiation of sepsis from other causes of SIRS, and in the stratification of risk in sepsis patients. The methods and compositions of the present invention can be used to facilitate the treatment of patients and the development of additional diagnostic and/or prognostic indicators and therapies.

In various aspects, the invention relates to materials and procedures for identifying markers that may be used to direct therapy in subjects; to using such markers in treating a patient and/or to monitor the course of a treatment regimen; to using such markers to identify subjects at risk for one or more adverse outcomes related to SIRS; and for screening compounds and pharmaceutical compositions that might provide a benefit in treating or preventing such conditions.

In a first aspect, the invention relates to methods for determining an appropriate treatment regimen for a subject, preferably one suffering from or suspected to suffer from SIRS, sepsis, severe sepsis, septic shock and/or MODS. These methods comprise analyzing a test sample obtained from a subject for the presence or amount of one or more markers related to blood pressure regulation, markers related to coagulation and hemostasis, markers related to apoptosis, and/or markers related to inflammation. The presence or amount of the marker(s) measured may be compared, individually or in groups, to levels of the marker(s) selected to rule in or out one or more particular treatments. Preferred treatments to be ruled in or out are those used to treat SIRS, sepsis, severe sepsis, septic shock and/or MODS, most preferably early sepsis therapy regimens as defined hereinafter.

In a related aspect, the invention relates to methods for determining a prognosis for a subject. These methods comprise analyzing a test sample obtained from a subject for the presence or amount of one or more markers related to blood pressure regulation, markers related to coagulation and hemostasis, markers related to apoptosis, and/or markers related to inflammation. The presence or amount of the marker(s) measured may be compared, individually or in groups, to levels of the marker(s) indicative of a future outcome, either positive (e.g., that the subject is likely to live) or negative (e.g., that the subject is at an increased risk of death).

In certain embodiments, a plurality of such markers, comprising 2, 3, 4, 5, 6, 7, 8, 9, 10, 15, 20, or more or individual markers, are combined into a marker panel. Exemplary markers for inclusion in such panels are described in detail hereinafter.

In certain embodiments, concentrations of the individual markers can each be compared to a level (a “threshold”) that is preselected to rule in or out one or more particular diagnoses, prognoses, and/or therapy regimens. By correlating each of the subject's selected marker levels to thresholds for each marker of interest, a subject may be assigned to or excluded from one or more particular therapies. Similarly, by correlating the subject's marker levels to prognostic thresholds for each marker, the probability that the subject will suffer one or more future adverse outcomes may be determined.

In other embodiments, particular thresholds for one or more markers in a panel are not relied upon to determine if a profile of marker levels obtained from a subject are correlated to a particular therapy or prognosis. Rather, the present invention may utilize an evaluation of the entire profile of markers to provide a single result value (e.g., a “panel response” value expressed either as a numeric score or as a percentage risk). In such embodiments, an increase, decrease, or other change (e.g., slope over time) in a certain subset of markers may be sufficient to indicate a particular outcome in one patient, while an increase, decrease, or other change in a different subset of markers may be sufficient to indicate the same or a different outcome in another patient. Methods for performing such analyses are described hereinafter.

In yet other embodiments, multiple determinations of one or more markers can be made, and a temporal change in the markers can be used to rule in or out one or more particular therapies and/or prognoses. For example, one or more markers may be determined at an initial time, and again at a second time, and the change (or lack thereof) in the marker level(s) over time determined. In such embodiments, an increase in the marker from the initial time to the second time may be indicative of a particular prognosis, rule in or out a particular therapy, etc. Likewise, a decrease in the marker from the initial time to the second time may be indicative of a particular prognosis, rule in or out a particular therapy, etc. In such a panel, the markers need not change in concert with one another. Temporal changes in one or more markers may also be used together with single time point marker levels to increase the discriminating power of marker panels. In yet another alternative, a “panel response” may be treated as a marker, and temporal changes in the panel response may be indicative of a particular prognosis, rule in or out a particular therapy, etc.

In a particularly preferred embodiment, the presence or amount of one or more markers related to blood pressure regulation in a sample are used prognostically to determine a risk of a future complication related to SIRS, sepsis, severe sepsis, septic shock and/or MODS. In these embodiments, a preferred marker related to blood pressure regulation is BNP, or NT-proBNP, or a marker related thereto. Similarly, in another particularly preferred embodiment, the presence or amount of one or more markers related to inflammation in a sample are used prognostically to determine a risk of a future complication related to SIRS, sepsis, severe sepsis, septic shock and/or MODS. In these embodiments, a preferred marker related to blood pressure regulation is BNP, or NT-proBNP, or a marker related thereto. As described hereinafter, such methods may be used to determine an outcome risk in a subject, and this risk used to guide treatment decisions for that subject.

As discussed in detail herein, preferably a plurality of markers are combined to increase the predictive value of the analysis in comparison to that obtained from the markers individually. Such panels may comprise 2, 3, 4, 5, 6, 7, 8, 9, 10, 15, 20, or more or individual markers. The skilled artisan will also understand that diagnostic markers, differential diagnostic markers, prognostic markers, time of onset markers, etc., may be combined in a single assay or device. For example, certain markers measured by a device or instrument may be used provide a prognosis, while a different set of markers measured by the device or instrument may rule in and/or out particular therapies; each of these sets of markers may comprise unique markers, or may include markers that overlap with one or both of the other sets. Markers may also be commonly used for multiple purposes by, for example, applying a different set of analysis parameters (e.g., different midpoint, linear range window and/or weighting factor) to the marker(s) for the different purpose(s).

In certain embodiments, one or more markers are correlated to a therapy, prognosis, condition or disease by merely the presence or absence of the indicator(s). In other embodiments, threshold level(s) of a diagnostic or prognostic indicator(s) can be established, and the level of the indicator(s) in a patient sample can simply be compared to the threshold level(s). The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al., Radiology 143: 29-36 (1982).

In certain embodiments, markers and/or marker panels are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to ±5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to ±5% of a given measurement.

In the case of an odds ratio, a value of 1 indicates that a positive result is equally likely among subjects in both the “diseased” and “control” groups; a value greater than 1 indicates that a positive result is more likely in the diseased group, and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to ±5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to ±5% of a given measurement.

While exemplary panels are described herein, one or more markers may be replaced, added, or subtracted from these exemplary panels while still providing clinically useful results. Panels may comprise both specific markers of a disease (e.g., markers that are increased or decreased in bacterial infection, but not in other disease states) and/or non-specific markers (e.g., markers that are increased or decreased due to inflammation, regardless of the cause; markers that are increased or decreased due to changes in hemostasis, regardless of the cause, etc.). While certain markers may not individually be definitive in the methods described herein, a particular “fingerprint” pattern of changes may, in effect, act as a specific indicator of disease state. As discussed above, that pattern of changes may be obtained from a single sample, or may optionally consider temporal changes in one or more members of the panel (or temporal changes in a panel response value).

Particularly preferred marker panels comprise, for example, one or more first marker(s) selected from the group consisting of atrial natriuretic peptide (“ANP), NT-proANP, pro-ANP, B-type natriuretic peptide (“BNP”), NT-pro BNP, pro-BNP C-type natriuretic peptide, NT-proCNP, pro-CNP, urotensin II, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, renin, and urodilatin, or markers related thereto (referred to collectively as “markers related to blood pressure regulation”); and one or more second markers selected from the group consisting of acute phase reactants, cell adhesion molecules such as vascular cell adhesion molecule (“VCAM”), soluble intercellular adhesion molecule-1 (“sICAM-1”), soluble intercellular adhesion molecule-2 (“sICAM-2”), and soluble intercellular adhesion molecule-3 (“sICAM-3”), C-reactive protein, HMG-1 (also known as HMGB1), interleukins such as IL-1β, IL-6, IL-8, IL-10, and IL-22, chemokines such as the CXCL and CCL families (e.g., CXCL6, CXCL13, CXCL16, CCL8, CCL20, CCL23, and CCL26), interleukin-1 receptor agonist, monocyte chemotactic protein-1, lipocalin-type prostaglandin D synthase, mast cell tryptase, eosinophil cationic protein, KL-6, haptoglobin, tumor necrosis factor a, tumor necrosis factor β, soluble Fas ligand, soluble Fas (Apo-1), TRAIL, TWEAK, TREM-1, fibronectin, macrophage migration inhibitory factor (MIF), and vascular endothelial growth factor (“VEGF”), or markers related thereto (referred to collectively as “markers related to inflammation”). The term “related markers” is defined hereinafter.

One or more additional markers selected from the group consisting of plasmin, fibrinogen, D-dimer, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, von Willebrand factor, tissue factor, and thrombus precursor protein, or markers related thereto (referred to collectively as “markers related to coagulation and hemostasis”) may be included in the panels of the present invention.

Preferred marker(s) related to apoptosis for use in the methods described herein may also be used in the methods described herein, including for example, one or more marker(s) selected from the group consisting of spectrin, cathepsin D, caspase-3, cytochrome c, s-acetyl glutathione, and ubiquitin fusion degradation protein 1 homolog, or markers related thereto.

In addition to those acute phase reactants listed above as “markers related to inflammation,” one or more markers related to inflammation may also be selected from the group of acute phase reactants consisting of hepcidin, HSP-60, HSP-65, HSP-70, asymmetric dimethylarginine (an endogenous inhibitor of nitric oxide synthase), matrix metalloproteins 11, 3, and 9, defensin HBD 1, defensin HBD 2, serum amyloid A, oxidized LDL, insulin like growth factor, transforming growth factor β, inter-α-inhibitors, e-selectin, glutathione-S-transferase, hypoxia-inducible factor-1α, inducible nitric oxide synthase (“I-NOS”), intracellular adhesion molecule, lactate dehydrogenase, matrix metalloproteinase-9 (“MMP-9”), monocyte chemoattractant peptide-1 (“MCP-1”), n-acetyl aspartate, prostaglandin E2, receptor activator of nuclear factor (“RANK”) ligand, TNF receptor superfamily member 1A, and cystatin C, or markers related thereto. Additional markers related to blood pressure regulation, to inflammation, and to coagulation and hemostasis are described hereinafter.

Likewise, one or more markers related to reactive oxygen species may also be measured as part of such a panel. The marker(s) may be selected from the group consisting of superoxide dismutase, glutathione, α-tocopherol, ascorbate, inducible nitric oxide synthase, lipid peroxidation products, nitric oxide, myeloperoxidase, and breath hydrocarbons (preferably ethane), or markers related thereto.

Additional markers and/or marker classes may be added to such panels to provide further ability to discriminate amongst diseases. For example, the inflammatory response and resulting effects on capillaries and reduced oxygenation of tissues implicate one or more markers related to the acute phase response, one or more markers related to vascular tissues, and one or more tissue-specific markers (e.g., neural-specific markers such as CK-BB), the levels of which are increased in ischemic conditions. Thus, one or more markers selected from the group consisting of α-2 actin, basic calponin 1, β-1 integrin, acidic calponin, caldesmon, cysteine rich protein-2 (“CRP 2” or “CSRP 2”), elastin, fibrillin 1, latent transforming growth factor beta binding protein 4 (“LTBP 4”), smooth muscle myosin, smooth muscle myosin heavy chain, and transgelin, or markers related thereto (referred to collectively as “markers related to vascular tissue”) may be included in such a panel. Additional markers and marker classes are described hereinafter.

These markers may be combined in various combinations. For example, preferred panels may include 1, 2, 3, 4, 5, 6, 7, 8, 9, 10, or more markers selected from the group consisting of CRP, caspase-3, CK-BB, IL-1β, IL-1ra, IL-6, IL-8, HMG-1, TNFα, MIF, MCP-1, MMP-9, Fas ligand, soluble Fas (Apo-1), TRAIL, TWEAK, ANP, pro-ANP, BNP, CNP, pro-BNP, pro-CNP, NT-pro-BNP, tissue factor, von Willebrand factor, vWF-A1, vWF-integrin binding domain, and vWF-A3, or markers related thereto. As discussed herein, these markers may be measured at a single time point, and/or may be measured at multiple time points for calculation of a change in the marker level(s) over time.

In a related aspect, the present invention relates to methods for identifying marker panels for use in the foregoing methods. In developing a panel of markers useful in diagnosis, prognosis, and/or therapy, data for a number of potential markers may be obtained from a group of subjects by testing for the presence or level of certain markers. The group of subjects may then be divided into sets. For example, a first set includes subjects who have been confirmed as having a disease or, more generally, being in a first condition state. The confirmation of this condition state may be made through a more rigorous and/or expensive testing, such as culture of a tissue sample for organisms in sepsis. Hereinafter, subjects in this first set will be referred to as “diseased”. A second set of subjects is selected from those who do not fall within the first set. Subjects in this second set will hereinafter be referred to as “non-diseased”.

The data obtained from subjects in these sets includes levels of a plurality of markers. Preferably, data for the same set of markers is available for each patient. Exemplary markers are described herein. Actual known relevance of the marker(s) to the disease of interest is not required. Methods for comparing these subject sets for relevance of one or more markers is described hereinafter. Embodiments of the methods and systems described herein may be used to determine which of the candidate markers are most relevant to the diagnosis of the disease or condition or of a given prognosis.

In yet a further aspect, the invention relates to devices to perform one or more of the methods described herein. Such devices preferably contain a plurality of diagnostic zones, each of which is related to a particular marker of interest. Such devices may be referred to as “arrays” or “microarrays.” Following reaction of a sample with the devices, a signal is generated from the diagnostic zone(s), which may then be correlated to the presence or amount of the markers of interest. Numerous suitable devices are known to those of skill in the art.

### BRIEF DESCRIPTION OF THE FIGURES

FIGS. 1 and 2 show a time course for the levels of various markers measured in samples from sepsis patients, relative to the time of death for those patients, in subjects receiving early goal-directed therapy (FIG. 1) and conventional sepsis therapy (FIG. 2).

### DETAILED DESCRIPTION OF THE INVENTION

The present invention relates to methods and compositions for symptom-based differential diagnosis, prognosis, and determination of treatment regimens in subjects. In particular, the invention relates to methods and compositions selected to rule in or out SIRS, or for differentiating sepsis, severe sepsis, septic shock, and/or MODS from each other and/or from non-infectious SIRS.

Patients presenting for medical treatment often exhibit one or a few primary observable changes in bodily characteristics or functions that are indicative of disease. Often, these “symptoms” are nonspecific, in that a number of potential diseases can present the same observable symptom or symptoms. In the case of SIRS, the condition exists, by definition, whenever two or more of the following symptoms are present:
a temperature >38° C. or <36° C.;
a heart rate of >90 beats per minute (tachycardia);
a respiratory rate of >20 breaths per minute (tachypnea) or a PₐCO₂<4.3 kPa; and a white blood cell count >12,000 per mm³, <4,000 per mm³, or >10% immature (band) forms.

The present invention describes methods and compositions that can assist in the differential diagnosis of one or more nonspecific symptoms by providing diagnostic markers that are designed to rule in or out one, and preferably a plurality, of possible etiologies for the observed symptoms. Symptom-based differential diagnosis described herein can be achieved using panels of diagnostic markers designed to distinguish between possible diseases that underlie a nonspecific symptom observed in a patient.

Definitions

The term “therapy regimen” refers to one or more interventions made by a caregiver in hopes of treating a disease or condition. The term “early sepsis therapy regimen” refers to a set of supportive therapies designed to reduce the risk of mortality when administered within the initial 24 hours, more preferably within the initial 12 hours, and most preferably within the initial 6 hours or earlier, of assigning a diagnosis of SIRS, sepsis, severe sepsis, septic shock, or MODS to a subject. Such supportive therapies comprise a spectrum of treatments including resuscitation, fluid delivery, vasopressor administration, inotrope administration, steroid administration, blood product administration, and/or sedation. See, e.g., Dellinger et al., Crit. Care Med. 32: 858-873, 2004, and Rivers et al., N. Engl. J. Med. 345: 1368-1377, 2001 (providing a description of “early goal directed therapy” as that term is used herein), each of which is hereby incorporated by reference. Preferably, such an early sepsis therapy regimen comprises one or more, and preferably a plurality, of the following therapies:
maintenance of a central venous pressure of 8-12 mm Hg, preferably by administration of crystalloids and/or colloids as necessary;
maintenance of a mean arterial pressure of ≧65 mm Hg, preferably by administration of vasopressors and/or vasodilators as necessary; maintenance of a central venous oxygen saturation of ≧70%, preferably by administration of transfused red blood cells to a hematocrit of at least 30% and/or administration of dobutamine as necessary; and
administration of mechanical ventilation as necessary.

The term “marker” as used herein refers to proteins, polypeptides, glycoproteins, proteoglycans, lipids, lipoproteins, glycolipids, phospholipids, nucleic acids, carbohydrates, etc. or small molecules to be used as targets for screening test samples obtained from subjects. “Proteins or polypeptides” used as markers in the present invention are contemplated to include any fragments thereof, in particular, immunologically detectable fragments.

The term “related marker” as used herein refers to one or more fragments of a particular marker or its biosynthetic parent that may be detected as a surrogate for the marker itself or as independent markers. For example, human BNP is derived by proteolysis of a 108 amino acid precursor molecule, referred to hereinafter as BNP₁₋₁₀₈. Mature BNP, or “the BNP natriuretic peptide,” or “BNP-32” is a 32 amino acid molecule representing amino acids 77-108 of this precursor, which may be referred to as BNP₇₇₋₁₀₈. The remaining residues 1-76 are referred to hereinafter as BNP₁₋₇₆. Additionally, related markers may be the result of covalent modification of the parent marker, for example by oxidation of methionine residues, ubiquitination, cysteinylation, nitrosylation, glycosylation, complex formation, differential splicing, etc.

The sequence of the 108 amino acid BNP precursor pro-BNP (BNP₁₋₁₀₈) is as follows, with mature BNP (BNP₇₇₋₁₀₈) underlined:

| (SEQ ID NO: 1) | |
|---|---|
| HPLGSPGSAS DLETSGLQEQ RNHLQGKLSE LQVEQTSLEP LQESPRPTGV | 50 |
| WKSREVATEG IRGHRKMVLY TLRAPRSPKM VQGSGCFGRK MDRISSSSGL | 100 |
| GCKVLRRH. | 108 |

BNP₁₋₁₀₈ is synthesized as a larger precursor pre-pro-BNP having the following sequence (with the “pre” sequence shown in bold):

| (SEQ ID NO: 2) | |
|---|---|
| MDPQTAPSRA LLLLLFLHLA FLGGRSHPLG SPGSASDLET SGLQEQRNHL | 50 |
| QGKLSELQVE QTSLEPLQES PRPTGVWKSR EVATEGIRGH RKMVLYTLRA | 100 |
| PRSPKMVQGS GCFGRKMDRI SSSSGLGCKV LRRH | 134 |

While mature BNP itself may be used as a marker in the present invention, the prepro-BNP, BNP₁₋₁₀₈ and BNP₁₋₇₆ molecules represent BNP-related markers that may be measured either as surrogates for mature BNP or as markers in and of themselves. In addition, one or more fragments of these molecules, including BNP-related polypeptides selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈ may also be present in circulation. In addition, natriuretic peptide fragments, including BNP fragments, may comprise one or more oxidizable methionines, the oxidation of which to methionine sulfoxide or methionine sulfone produces additional BNP-related markers. See, e.g., U.S. Pat. No. 10/419,059, filed Apr. 17, 2003, which is hereby incorporated by reference in its entirety including all tables, figures and claims.

Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result. In addition, individual antibodies that distinguish amongst a plurality of marker fragments may be individually employed to separately detect the presence or amount of different fragments. The results of this individual detection may provide a more accurate prognostic or diagnostic result than detecting the plurality of fragments in a single assay. For example, different weighting factors may be applied to the various fragment measurements to provide a more accurate estimate of the amount of natriuretic peptide originally present in the sample.

In a similar fashion, many of the markers described herein are synthesized as larger precursor molecules, which are then processed to provide mature marker; and/or are present in circulation in the form of fragments of the marker. Thus, “related markers” to each of the markers described herein may be identified and used in an analogous fashion to that described above for BNP.

Removal of polypeptide markers from the circulation often involves degradation pathways. Moreover, inhibitors of such degradation pathways may hold promise in treatment of certain diseases. See, e.g., Trindade and Rouleau, Heart Fail. Monit. 2: 2-7, 2001. However, the measurement of the polypeptide markers has focused generally upon measurement of the intact form without consideration of the degradation state of the molecules. Assays may be designed with an understanding of the degradation pathways of the polypeptide markers and the products formed during this degradation, in order to accurately measure the biologically active forms of a particular polypeptide marker in a sample. The unintended measurement of both the biologically active polypeptide marker(s) of interest and inactive fragments derived from the markers may result in an overestimation of the concentration of biologically active form(s) in a sample.

The failure to consider the degradation fragments that may be present in a clinical sample may have serious consequences for the accuracy of any diagnostic or prognostic method. Consider for example a simple case, where a sandwich immunoassay is provided for BNP, and a significant amount (e.g., 50%) of the biologically active BNP that had been present has now been degraded into an inactive form. An immunoassay formulated with antibodies that bind a region common to the biologically active BNP and the inactive fragment(s) will overestimate the amount of biologically active BNP present in the sample by 2-fold, potentially resulting in a “false positive” result. Overestimation of the biologically active form(s) present in a sample may also have serious consequences for patient management. Considering the BNP example again, the BNP concentration may be used to determine if therapy is effective (e.g., by monitoring BNP to see if an elevated level is returning to normal upon treatment). The same “false positive” BNP result discussed above may lead the physician to continue, increase, or modify treatment because of the false impression that current therapy is ineffective.

Likewise, it may be necessary to consider the complex state of one or more markers described herein. For example, troponin exists in muscle mainly as a “ternary complex” comprising three troponin polypeptides (T, I and C). But troponin I and troponin T circulate in the blood in forms other than the I/T/C ternery complex. Rather, each of (i) free cardiac-specific troponin I, (ii) binary complexes (e.g., troponin I/C complex), and (iii) ternary complexes all circulate in the blood. Furthermore, the “complex state” of troponin I and T may change over time in a patient, e.g., due to binding of free troponin polypeptides to other circulating troponin polypeptides. Immunoassays that fail to consider the “complex state” of troponin may not detect all of the cardiac-specific isoform of interest.

Preferably, the methods described hereinafter utilize one or more markers that are derived from the subject. The term “subject-derived marker” as used herein refers to protein, polypeptide, phospholipid, nucleic acid, prion, glycoprotein, proteoglycan, glycolipid, lipid, lipoprotein, carbohydrate, or small molecule markers that are expressed or produced by one or more cells of the subject. The presence, absence, amount, or change in amount of one or more markers may indicate that a particular disease is present, or may indicate that a particular disease is absent. Additional markers may be used that are derived not from the subject, but rather that are expressed by pathogenic or infectious organisms that are correlated with a particular disease. Such markers are preferably protein, polypeptide, phospholipid, nucleic acid, prion, or small molecule markers that identify the infectious diseases described above.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

As used herein, a “plurality” as used herein refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are “patients,” i.e., living humans that are receiving medical care for a disease or condition. This includes persons with no defined illness who are being investigated for signs of pathology.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, amount, or change in amount of which is indicative of the presence, severity, or absence of the condition.

Similarly, a prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high level in samples obtained from such patients, the level may signal that the patient is at an increased probability for experiencing a future stroke in comparison to a similar patient exhibiting a lower marker level. A level or a change in level of a prognostic indicator, which in turn is associated with an increased probability of morbidity or death, is referred to as being “associated with an increased predisposition to an adverse outcome” in a patient. Preferred prognostic markers can predict the onset of delayed neurologic deficits in a patient after stroke, or the chance of future stroke.

The term “correlating,” as used herein in reference to the use of markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a profile of marker levels are correlated to a global probability or a particular outcome using ROC curves.

The term “discrete” as used herein refers to areas of a surface that are non-contiguous. That is, two areas are discrete from one another if a border that is not part of either area completely surrounds each of the two areas.

The term “independently addressable” as used herein refers to discrete areas of a surface from which a specific signal may be obtained.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The term “specifically binds” is not intended to indicate that an antibody binds exclusively to its intended target. Rather, an antibody “specifically binds” if its affinity for its intended target is about 5-fold greater when compared to its affinity for a non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, Specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):
where
r=moles of bound ligand/mole of receptor at equilibrium;
c=free ligand concentration at equilibrium;
K=equilibrium association constant; and
n=number of ligand binding sites per receptor molecule
   By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

Identification of Marker Panels

In accordance with the present invention, there are provided methods and systems for the identification of one or more markers useful in diagnosis, prognosis, and/or determining an appropriate therapeutic course. Suitable methods for identifying markers useful for such purposes are described in detail in U.S. Provisional Patent Application No. 60/436,392 filed Dec. 24, 2002, PCT application US03/41426 filed Dec. 23, 2003, U.S. patent application Ser. No. 10/331,127 filed Dec. 27, 2002, and PCT application No. US03/41453, each of which is hereby incorporated by reference in its entirety, including all tables, figures, and claims.

One skilled in the art will also recognize that univariate analysis of markers can be performed and the data from the univariate analyses of multiple markers can be combined to form panels of markers to differentiate different disease conditions. Such methods include multiple linear regression, determining interaction terms, stepwise regression, etc.

In developing a panel of markers, data for a number of potential markers may be obtained from a group of subjects by testing for the presence or level of certain markers. The group of subjects is divided into two sets. The first set includes subjects who have been confirmed as having a disease, outcome, or, more generally, being in a first condition state. For example, this first set of patients may be those diagnosed with SIRS, sepsis, severe sepsis, septic shock and/or MODS that died as a result of that disease. Hereinafter, subjects in this first set will be referred to as “diseased.”

The second set of subjects is simply those who do not fall within the first set. Subjects in this second set will hereinafter be referred to as “non-diseased”. Preferably, the first set and the second set each have an approximately equal number of subjects. This set may be normal patients, and/or patients suffering from another cause of SIRS, and/or that lived to a particular endpoint of interest.

The data obtained from subjects in these sets preferably includes levels of a plurality of markers. Preferably, data for the same set of markers is available for each patient. This set of markers may include all candidate markers that may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a single marker often is incapable of definitively identifying a subject as falling within a first or second group in a prospective fashion. For example, if a patient is measured as having a marker level that falls within an overlapping region in the distribution of diseased and n6n-diseased subjects, the results of the test may be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As discussed above, the measurement of the level of a single marker may have limited usefulness, e.g., it may be non-specifically increased due to inflammation. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. In a preferred embodiment, the panel response (R) for each subject (j) is expressed as:
Rⱼ=ΣwᵢI_{i,j}
where i is the marker index, j is the subject index, wᵢ is the weighting coefficient for marker i, I is the indicator value to which the marker level for marker i is mapped for subject j, and Σ is the summation over all candidate markers i. This panel response value may be referred to as a “panel index.”

One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. Certain markers may be then be changed or even eliminated from the panel, and the process repeated until a satisfactory result is obtained. The effective contribution of each marker in the panel may be determined to identify the relative importance of the markers. In one embodiment, the weighting coefficients resulting from the optimization process may be used to determine the relative importance of each marker. The markers with the lowest coefficients may be eliminated or replaced.

In certain cases, the lower weighting coefficients may not be indicative of a low importance. Similarly, a higher weighting coefficient may not be indicative of a high importance. For example, the optimization process may result in a high coefficient if the associated marker is irrelevant to the diagnosis. In this instance, there may not be any advantage that will drive the coefficient lower. Varying this coefficient may not affect the value of the objective function.

To allow a determination of test accuracy, a “gold standard” test criterion may be selected which allows selection of subjects into two or more groups for comparison by the foregoing methods. In the case of sepsis, this gold standard may be recovery of organisms from culture of blood, urine, pleural fluid, cerebrospinal fluid, peritoneal fluid, synnovial fluid, sputum, or other tissue specimens. This implies that those negative for the gold standard are free of sepsis; however, as discussed above, 50% or more of patients exhibiting strong clinical evidence of sepsis are negative on culture. In this case, those patients showing clinical evidence of sepsis but a negative gold standard result may be omitted from the comparison groups. Alternatively, an initial comparison of confirmed sepsis subjects may be compared to normal healthy control subjects. In the case of a prognosis, mortality is a common test criterion.

Measures of test accuracy may be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, suitable tests may exhibit one or more of the following results on these various measures:
at least 75% sensitivity, combined with at least 75% specificity;
ROC curve area of at least 0.6, more preferably 0.7, still more preferably at least 0.8, even more preferably at least 0.9, and most preferably at least 0.95; and/or
a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

Exemplary Marker Panels

In a preferred embodiment, the following discussion considers BNP, representative of one or more markers related to blood pressure regulation, and C-reactive protein, representative of one or more markers related to inflammation, for inclusion in a marker panel for use in the methods described herein. Additional markers that may be included are one or more markers related to coagulation and hemostasis, and/or one or more markers related to apoptosis, and/or one or more markers related to vascular tissue, and/or one or more acute phase reactants. Additional suitable marker classes are described hereinafter.

BNP

B-type natriuretic peptide (BNP), also called brain-type natriuretic peptide is a 32 amino acid, 4 kDa peptide that is involved in the natriuresis system to regulate blood pressure and fluid balance. Bonow, R. O., Circulation 93:1946-1950 (1996). The precursor to BNP is synthesized as a 108-amino acid molecule, referred to as “proBNP,” that is proteolytically processed into a 76-amino acid N-terminal peptide (amino acids 1-76), referred to as “NT-proBNP” and the 32-amino acid mature hormone, referred to as BNP or BNP 32 (amino acids 77-108). ProBNP itself is synthesized as a larger precursor. It has been suggested that each of these species—NT-proBNP, BNP-32, and proBNP—can circulate in human plasma. Tateyama et al., Biochem. Biophys. Res. Commun. 185: 760-7 (1992); Hunt et al., Biochem. Biophys. Res. Commun. 214: 1175-83 (1995). The 2 forms, proBNP and NT-proBNP, and peptides which are derived from BNP and/or its biosynthetic precursors are collectively described as markers related to or associated with BNP. Preferred markers related to BNP include pro-BNP, NT-proBNP, and fragments such as BNP₃₋₁₀₈, BNP₃₋₁₀₆, BNP₇₉₋₁₀₈, and BNP₇₉₋₁₀₆.

Elevations of BNP are associated with raised atrial and pulmonary wedge pressures, reduced ventricular systolic and diastolic function, left ventricular hypertrophy, and myocardial infarction. Sagnella, G. A., Clinical Science 95: 519-29 (1998). Furthermore, there are numerous reports of elevated BNP concentration associated with congestive heart failure and renal failure. Thus, BNP levels in a patient may be indicative of several possible underlying causes of dyspnea.

C-Reactive Protein

C-reactive protein (CRP) is a homopentameric Ca²⁺-binding acute phase protein with 21 kDa subunits that is involved in host defense. CRP preferentially binds to phosphorylcholine, a common constituent of microbial membranes. Phosphorylcholine is also found in mammalian cell membranes, but it is not present in a form that is reactive with CRP. The interaction of CRP with phosphorylcholine promotes agglutination and opsonization of bacteria, as well as activation of the complement cascade, all of which are involved in bacterial clearance. Furthermore, CRP can interact with DNA and histones, and it has been suggested that CRP is a scavenger of nuclear material released from damaged cells into the circulation (Robey, F. A. et al., J. Biol. Chem. 259:7311-7316, 1984). CRP synthesis is induced by I1-6, and indirectly by IL-1, since IL-1 can trigger the synthesis of IL-6 by Kupffer cells in the hepatic sinusoids. The normal plasma concentration of CRP is <3 μg/ml (30 nM) in 90% of the healthy population, and <10 μg/ml (100 nM) in 99% of healthy individuals. Plasma CRP concentrations can be measured by rate nephelometry or ELISA. The concentration of CRP will be elevated in the plasma from individuals with any condition that may elicit an acute phase response, such as infection, surgery, trauma, myocardial infarction, and stroke. CRP is a secreted protein that is released into the bloodstream soon after synthesis. CRP synthesis is upregulated by IL-6, and the plasma CRP concentration is significantly elevated within 6 hours of stimulation (Biasucci, L. M. et al., Am. J. Cardiol. 77:85-87, 1996). The plasma CRP concentration peaks approximately 50 hours after stimulation, and begins to decrease with a half-life of approximately 19 hours in the bloodstream (Biasucci, L. M. et al., Am. J. Cardiol. 77:85-87, 1996).

A detailed analysis of this exemplary marker panel is provided in the following examples. The skilled artisan will readily acknowledge that other markers may be substituted in or added to this marker panel to further discriminate the causes of SIRS in accordance with the methods for identification and use of diagnostic markers described herein. Additional suitable markers are described in the following sections.

A panel consisting of the markers referenced herein may be constructed to provide relevant information related to the diagnosis of interest. Such a panel may be constructed using 1, 2, 3, 4, 5, 6, 7, 8, 9, 10, 11, 12, 13, 14, 15, 16, 17, 18, 19, 20, or more individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The following provides a brief discussion of additional exemplary markers for use in identifying suitable marker panels by the methods described herein.

(i) Exemplary Markers Related to Blood Pressure Regulation

A-type natriuretic peptide (ANP) (also referred to as atrial natriuretic peptide or cardiodilatin (Forssmann et al Histochem Cell Biol 110: 335-357, 1998) is a 28 amino acid peptide that is synthesized, stored, and released atrial myocytes in response to atrial distension, angiotensin II stimulation, endothelin, and sympathetic stimulation (beta-adrenoceptor mediated). ANP is synthesized as a precursor molecule (pro-ANP) that is converted to an active form, ANP, by proteolytic cleavage and also forming N-terminal ANP (1-98). N-terminal ANP and ANP have been reported to increase in patients exhibiting atrial fibrillation and heart failure (Rossi et al. Journal of the American College of Cardiology 35: 1256-62, 2000). In addition to atrial natriuretic peptide (ANP99-126) itself, linear peptide fragments from its N-terminal prohormone segment have also been reported to have biological activity. As the skilled artisan will recognize, however, because of its relationship to ANP, the concentration of N-terminal ANP molecule can also provide diagnostic or prognostic information in patients. The phrase “marker related to ANP or ANP related peptide” refers to any polypeptide that originates from the pro-ANP molecule (1-126), other than the 28-amino acid ANP molecule itself. Proteolytic degradation of ANP and of peptides related to ANP have also been described in the literature and these proteolytic fragments are also encompassed it the term “ANP related peptides.”

Elevated levels of ANP are found during hypervolemia, atrial fibrillation and congestive heart failure. ANP is involved in the long-term regulation of sodium and water balance, blood volume and arterial pressure. This hormone decreases aldosterone release by the adrenal cortex, increases glomerular filtration rate (GFR), produces natriuresis and diuresis (potassium sparing), and decreases renin release thereby decreasing angiotensin II. These actions contribute to reductions in blood volume and therefore central venous pressure (CVP), cardiac output, and arterial blood pressure. Several isoforms of ANP have been identified, and their relationship to stroke incidence studied. See, e.g., Rubatu et al., Circulation 100:1722-6, 1999; Estrada et al., Am. J. Hypertens. 7:1085-9, 1994.

Chronic elevations of ANP appear to decrease arterial blood pressure primarily by decreasing systemic vascular resistance. The mechanism of systemic vasodilation may involve ANP receptor-mediated elevations in vascular smooth muscle cGMP as well as by attenuating sympathetic vascular tone. This latter mechanism may involve ANP acting upon sites within the central nervous system as well as through inhibition of norepinephrine release by sympathetic nerve terminals. ANP may be viewed as a counter-regulatory system for the renin-angiotensin system.

C-type natriuretic peptide (CNP) is a 22-amino acid peptide that is the primary active natriuretic peptide in the human brain; CNP is also considered to be an endothelium-derived relaxant factor, which acts in the same way as nitric oxide (NO) (Davidson et al., Circulation 93:1155-9, 1996). CNP is structurally related to Atrial natriuretic peptide (ANP) and B-type natriuretic peptide (BNP); however, while ANP and BNP are synthesized predominantly in the myocardium, CNP is synthesized in the vascular endothelium as a precursor (pro-CNP) (Prickett et al., Biochem. Biophys. Res. Commun. 286:513-7, 2001). CNP is thought to possess vasodilator effects on both arteries and veins and has been reported to act mainly on the vein by increasing the intracellular cGMP concentration in vascular smooth muscle cells.

Urotensin II is a peptide having the sequence Ala-Gly-Thr-Ala-Asp-Cys-Phe-Trp-Lys-Tyr-Cys-Val, with a disulfide bridge between Cys6 and Cys 11. Human urotensin 2 (UTN) is synthesized in a prepro form. Processed urotensin 2 has potent vasoactive and cardiostimulatory effects, acting on the G protein-linked receptor GPR14.

Vasopressin (arginine vasopressin, AVP; antidiuretic hormone, ADH) is a peptide hormone released from the posterior pituitary. Its primary function in the body is to regulate extracellular fluid volume by affecting renal handling of water. There are several mechanisms regulating release of AVP. Hypovolemia, as occurs during hemorrhage, results in a decrease in atrial pressure. Specialized stretch receptors within the atrial walls and large veins (cardiopulmonary baroreceptors) entering the atria decrease their firing rate when there is a fall in atrial pressure. Afferent from these receptors synapse within the hypothalamus; atrial receptor firing normally inhibits the release of AVP by the posterior pituitary. With hypovolemia or decreased central venous pressure, the decreased firing of atrial stretch receptors leads to an increase in AVP release. Hypothalamic osmoreceptors sense extracellular osmolarity and stimulate AVP release when osmolarity rises, as occurs with dehydration. Finally, angiotensin II receptors located in a region of the hypothalamus regulate AVP release—an increase in angiotensin II simulates AVP release.

AVP has two principle sites of action: kidney and blood vessels. The most important physiological action of AVP is that it increases water reabsorption by the kidneys by increasing water permeability in the collecting duct, thereby permitting the formation of a more concentrated urine. This is the antidiuretic effect of AVP. This hormone also constricts arterial blood vessels; however, the normal physiological concentrations of AVP are below its vasoactive range.

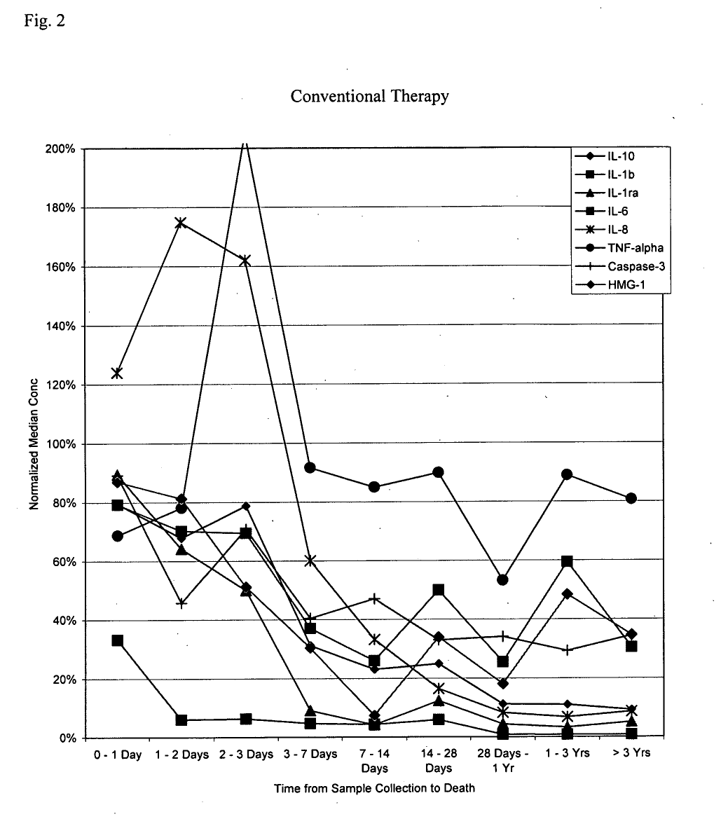

Calcitonin gene related peptide (CGRP) is a polypeptide of 37 amino acids that is a product of the calcitonin gene derived by alternative splicing of the precursor mRNA. The calcitonin gene (CALC-I) primary RNA transcript is processed into different mRNA segments by inclusion or exclusion of different exons as part of the primary transcript. Calcitonin-encoding mRNA is the main product of CALC-I transcription in C-cells of the thyroid, whereas CGRP-I mRNA (CGRP=calcitonin-gene-related peptide) is produced in nervous tissue of the central and peripheral nervous systems (FIG. 2.2.1) (9). In the third mRNA sequence, the calcitonin sequence is lost and alternatively the sequence of CGRP is encoded in the mRNA. CGRP is a markedly vasoactive peptide with vasodilatative properties. CGRP has no effect on calcium and phosphate metabolism and is synthesized predominantly in nerve cells related to smooth muscle cells of the blood vessels (149). ProCGRP, the precursor of CGRP, and PCT have partly identical N-terminal amino acid sequences.

Procalcitonin is a 116 amino acid (14.5 kDa) protein encoded by the Calc-1 gene located on chromosome 11p15.4. The Calc-1 gene produces two transcripts that are the result of alternative splicing events. Pre-procalcitonin contains a 25 amino acid signal peptide which is processed by C-cells in the thyrois to a 57 amino acid N-terminal fragment, a 32 amino acid calcitonin fragment, and a 21 amino acid katacalcin fragment. Procalcitonin is secreted intact as a glycosylated product by other body cells. Whicher et al., Ann. Clin. Biochem. 38: 483-93 (2001). Plasma procalcitonin has been identified as a marker of sepsis and its severity (Yukioka et al., Ann. Acad. Med. Singapore 30: 528-31 (2001)), with day 2 procalcitonin levels predictive of mortality (Pettila et al., Intensive Care Med. 28: 1220-25 (2002). Procalcitonin₃₋₁₁₆, a molecule related to procalcitonin as that term is defined herein, is also found in the circulation. See, e.g., U.S. Pat. 6,756,483.

Angiotensin II is an octapeptide hormone formed by renin action upon a circulating substrate, angiotensinogen, that undergoes proteolytic cleavage to from the decapeptide angiotensin I. Vascular endothelium, particularly in the lungs, has an enzyme, angiotensin converting enzyme (ACE), that cleaves off two amino acids to form the octapeptide, angiotensin II (AII).

All has several very important functions: Constricts resistance vessels (via AII receptors) thereby increasing systemic vascular resistance and arterial pressure; Acts upon the adrenal cortex to release aldosterone, which in turn acts upon the kidneys to increase sodium and fluid retention; Stimulates the release of vasopressin (antidiuretic hormone, ADH) from the posterior pituitary which acts upon the kidneys to increase fluid retention; Stimulates thirst centers within the brain; Facilitates norepinephrine release from sympathetic nerve endings and inhibits norepinephrine re-uptake by nerve endings, thereby enhancing sympathetic adrenergic function; and Stimulates cardiac hypertrophy and vascular hypertrophy.

Adrenomedullin (AM) is a 52-amino acid peptide which is produced in many tissues, including adrenal medulla, lung, kidney and heart (Yoshitomi et al., Clin. Sci. (Colch) 94:135-9, 1998). Intravenous administration of AM causes a long-lasting hypotensive effect, accompanied with an increase in the cardiac output in experimental animals. AM has been reported to enhance the stretch-induced release of ANP from the right atrium, but not to affect ventricular BNP expression. AM is synthesized as a precursor molecule (pro-AM). The N-terminal peptide processed from the AM precursor has also been reported to act as a hypotensive peptide (Kuwasako et al., Ann. Clin. Biochem. 36:622-8, 1999).

The endothelins are three related peptides (endothelin-1, endothelin-2, and endothelin-3) encoded by separate genes that are produced by vascular endothelium, each of which exhibit potent vasoconstricting activity. Endothelin-1 (ET-1) is a 21 amino acid residue peptide, synthesized as a 212 residue precursor (preproET-1), which contains a 17 residue signal sequence that is removed to provide a peptide known as big ET-1. This molecule is further processed by hydrolysis between trp21 and val22 by endothelin converting enzyme. Both big ET-1 and ET-1 exhibit biological activity; however the mature ET-1 form exhibits greater vasoconstricting activity (Brooks and Ergul, J. Mol. Endocrinol. 21:307-15, 1998). Similarly, endothelin-2 and endothelin-3 are also 21 amino acid residues in length, and are produced by hydrolysis of big endothelin-2 and big endothelin-3, respectively (Yap et al., Br. J. Pharmacol. 129:170-6, 2000; Lee et al., Blood 94:1440-50, 1999).

(ii) Exemplary Markers Related to Coagulation and Hemostasis

D-dimer is a crosslinked fibrin degradation product with an approximate molecular mass of 200 kDa. The normal plasma concentration of D-dimer is <150 ng/ml (750 pM). The plasma concentration of D-dimer is elevated in patients with acute myocardial infarction and unstable angina, but not stable angina. Hoffmeister, H. M. et al., Circulation 91: 2520-27 (1995); Bayes-Genis, A. et al., Thromb. Haemost. 81: 865-68 (1999); Gurfinkel, E. et al., Br. Heart J. 71: 151-55 (1994); Kruskal, J. B. et al., N. Engl. J. Med. 317: 1361-65 (1987); Tanaka, M. and Suzuki, A., Thromb. Res. 76: 289-98 (1994).

The plasma concentration of D-dimer also will be elevated during any condition associated with coagulation and fibrinolysis activation, including sepsis, stroke, surgery, atherosclerosis, trauma, and thrombotic thrombocytopenic purpura. D-dimer is released into the bloodstream immediately following proteolytic clot dissolution by plasmin. The plasma concentration of D-dimer can exceed 2 μg/ml in patients with unstable angina. Gurfinkel, E. et al., Br. Heart J. 71: 151-55 (1994). Plasma D-dimer is a specific marker of fibrinolysis and indicates the presence of a prothrombotic state associated with acute myocardial infarction and unstable angina. The plasma concentration of D-dimer is also nearly always elevated in patients with acute pulmonary embolism; thus, normal levels of D-dimer may allow the exclusion of pulmonary embolism. Egermayer et al., Thorax 53: 830-34 (1998).

Plasmin is a 78 kDa serine proteinase that proteolytically digests crosslinked fibrin, resulting in clot dissolution. The 70 kDa serine proteinase inhibitor α2-antiplasmin (α2AP) regulates plasmin activity by forming a covalent 1:1 stoichiometric complex with plasmin. The resulting ˜150 kDa plasmin-α2AP complex (PAP), also called plasmin inhibitory complex (PIC) is formed immediately after α2AP comes in contact with plasmin that is activated during fibrinolysis. The normal serum concentration of PAP is <1 μg/ml (6.9 nM). Elevations in the serum concentration of PAP can be attributed to the activation of fibrinolysis. Elevations in the serum concentration of PAP may be associated with clot presence, or any condition that causes or is a result of fibrinolysis activation. These conditions can include atherosclerosis, disseminated intravascular coagulation, acute myocardial infarction, surgery, trauma, unstable angina, stroke, and thrombotic thrombocytopenic purpura. PAP is formed immediately following proteolytic activation of plasmin. PAP is a specific marker for fibrinolysis activation and the presence of a recent or continual hypercoagulable state.

β-thromboglobulin (PTG) is a 36 kDa platelet a granule component that is released upon platelet activation. The normal plasma concentration of βTG is <40 ng/ml (1.1 nM). Plasma levels of β-TG appear to be elevated in patients with unstable angina and acute myocardial infarction, but not stable angina (De Caterina, R. et al., Eur. Heart J. 9:913-922, 1988; Bazzan, M. et al., Cardiologia 34, 217-220, 1989). Plasma β-TG elevations also seem to be correlated with episodes of ischemia in patients with unstable angina (Sobel, M. et al., Circulation 63:300-306, 1981). Elevations in the plasma concentration of βTG may be associated with clot presence, or any condition that causes platelet activation. These conditions can include atherosclerosis, disseminated intravascular coagulation, surgery, trauma, and thrombotic thrombocytopenic purpura, and stroke (Landi, G. et al., Neurology 37:1667-1671, 1987). βTG is released into the circulation immediately after platelet activation and aggregation. It has a biphasic half-life of 10 minutes, followed by an extended 1 hour half-life in plasma (Switalska, H. I. et al., J. Lab. Clin. Med. 106:690-700, 1985). Plasma βTG concentration is reportedly elevated dring unstable angina and acute myocardial infarction. Special precautions must be taken to avoid platelet activation during the blood sampling process. Platelet activation is common during regular blood sampling, and could lead to artificial elevations of plasma βTG concentration. In addition, the amount of βTG released into the bloodstream is dependent on the platelet count of the individual, which can be quite variable. Plasma concentrations of βTG associated with ACS can approach 70 ng/ml (2 nM), but this value may be influenced by platelet activation during the sampling procedure.

Platelet factor 4 (PF4) is a 40 kDa platelet a granule component that is released upon platelet activation. PF4 is a marker of platelet activation and has the ability to bind and neutralize heparin. The normal plasma concentration of PF4 is <7 ng/ml (175 pM). The plasma concentration of PF4 appears to be elevated in patients with acute myocardial infarction and unstable angina, but not stable angina (Gallino, A. et al., Am. Heart J. 112:285-290, 1986; Sakata, K. et al., Jpn. Circ. J. 60:277-284, 1996; Bazzan, M. et al., Cardiologia 34:217-220, 1989). Plasma PF4 elevations also seem to be correlated with episodes of ischemia in patients with unstable angina (Sobel, M. et al., Circulation 63:300-306, 1981). Elevations in the plasma concentration of PF4 may be associated with clot presence, or any condition that causes platelet activation. These conditions can include atherosclerosis, disseminated intravascular coagulation, surgery, trauma, thrombotic thrombocytopenic purpura, and acute stroke (Carter, A. M. et al., Arterioscler. Thromb. Vasc. Biol. 18:1124-1131, 1998). PF4 is released into the circulation immediately after platelet activation and aggregation. It has a biphasic half-life of 1 minute, followed by an extended 20 minute half-life in plasma. The half-life of PF4 in plasma can be extended to 20-40 minutes by the presence of heparin (Rucinski, B. et al., Am. J. Physiol. 251:H800-H807, 1986). Plasma PF4 concentration is reportedly elevated during unstable angina and acute myocardial infarction, but these studies may not be completely reliable. Special precautions must be taken to avoid platelet activation during the blood sampling process. Platelet activation is common during regular blood sampling, and could lead to artificial elevations of plasma PF4 concentration. In addition, the amount of PF4 released into the bloodstream is dependent on the platelet count of the individual, which can be quite variable. Plasma concentrations of PF4 associated with disease can exceed 100 ng/ml (2.5 nM), but it is likely that this value may be influenced by platelet activation during the sampling procedure.

Fibrinopeptide A (FPA) is a 16 amino acid, 1.5 kDa peptide that is liberated from amino terminus of fibrinogen by the action of thrombin. Fibrinogen is synthesized and secreted by the liver. The normal plasma concentration of FPA is <5 ng/ml (3.3 nM). The plasma FPA concentration is elevated in patients with acute myocardial infarction, unstable angina, and variant angina, but not stable angina (Gensini, G. F. et al., Thromb. Res. 50:517-525, 1988; Gallino, A. et al., Am. Heart J. 112:285-290, 1986; Sakata, K. et al., Jpn. Circ. J. 60:277-284, 1996; Theroux, P. et al., Circulation 75:156-162, 1987; Merlini, P. A. et al., Circulation 90:61-68, 1994; Manten, A. et al., Cardiovasc. Res. 40:389-395, 1998). Furthermore, plasma FPA may indicate the severity of angina (Gensini, G. F. et al., Thromb. Res. 50:517-525, 1988). Elevations in the plasma concentration of FPA are associated with any condition that involves activation of the coagulation pathway, including stroke, surgery, cancer, disseminated intravascular coagulation, nephrosis, sepsis, and thrombotic thrombocytopenic purpura. FPA is released into the circulation following thrombin activation and cleavage of fibrinogen. Because FPA is a small polypeptide, it is likely cleared from the bloodstream rapidly. FPA has been demonstrated to be elevated for more than one month following clot formation, and maximum plasma FPA concentrations can exceed 40 ng/ml in active angina (Gensini, G. F. et al., Thromb. Res. 50:517-525, 1988; Tohgi, H. et al., Stroke 21:1663-1667, 1990).

Platelet-derived growth factor (PDGF) is a 28 kDa secreted homo- or heterodimeric protein composed of the homologous subunits A and/or B (Mahadevan, D. et al., J. Biol. Chem. 270:27595-27600, 1995). PDGF is a potent mitogen for mesenchymal cells, and has been implicated in the pathogenesis of atherosclerosis. PDGF is released by aggregating platelets and monocytes near sites of vascular injury. The normal plasma concentration of PDGF is <0.4 ng/ml (15 pM). Plasma PDGF concentrations are higher in individuals with acute myocardial infarction and unstable angina than in healthy controls or individuals with stable angina (Ogawa, H. et al., Am. J. Cardiol. 69:453-456, 1992; Wallace, J. M. et al., Ann. Clin. Biochem. 35:236-241, 1998; Ogawa, H. et al., Coron. Artery Dis. 4:437-442, 1993). Changes in the plasma PDGF concentration in these individuals is most likely due to increased platelet and monocyte activation. Plasma PDGF is elevated in individuals with brain tumors, breast cancer, and hypertension (Kurimoto, M. et al., Acta Neurochir. (Wien) 137:182-187, 1995; Seymour, L. et al., Breast Cancer Res. Treat. 26:247-252, 1993; Rossi, E. et al., Am. J. Hypertens. 11:1239-1243, 1998). Plasma PDGF may also be elevated in any pro-inflammatory condition or any condition that causes platelet activation including surgery, trauma, sepsis, disseminated intravascular coagulation, and thrombotic thrombocytopenic purpura. PDGF is released from the secretory granules of platelets and monocytes upon activation. PDGF has a biphasic half-life of approximately 5 minutes and 1 hour in animals (Cohen, A. M. et al., J. Surg. Res. 49:447-452, 1990; Bowen-Pope, D. F. et al., Blood 64:458-469, 1984). The plasma PDGF concentration in ACS can exceed 0.6 ng/ml (22 pM) (Ogawa, H. et al., Am. J. Cardiol. 69:453-456, 1992). PDGF may be a sensitive and specific marker of platelet activation. In addition, it may be a sensitive marker of vascular injury, and the accompanying monocyte and platelet activation.

Prothrombin fragment 1+2 is a 32 kDa polypeptide that is liberated from the amino terminus of thrombin during thrombin activation. The normal plasma concentration of F1+2 is <32 ng/ml (1 nM). The plasma concentration of F1+2 is reportedly elevated in patients with acute myocardial infarction and unstable angina, but not stable angina, but the changes were not robust (Merlini, P. A. et al., Circulation 90:61-68, 1994). Other reports have indicated that there is no significant change in the plasma F1+2 concentration in cardiovascular disease (Biasucci, L. M. et al., Circulation 93:2121-2127, 1996; Manten, A. et al., Cardiovasc. Res. 40:389-395, 1998). The concentration of F1+2 in plasma can be elevated during any condition associated with coagulation activation, including stroke, surgery, trauma, thrombotic thrombocytopenic purpura, and disseminated intravascular coagulation. F1+2 is released into the bloodstream immediately upon thrombin activation. F1+2 has a half-life of approximately 90 minutes in plasma, and it has been suggested that this long half-life may mask bursts of thrombin formation (Biasucci, L. M. et al., Circulation 93:2121-2127, 1996).

P-selectin, also called granule membrane protein-140, GMP-140, PADGEM, and CD-62P, is a ˜140 kDa adhesion molecule expressed in platelets and endothelial cells. P-selectin is stored in the alpha granules of platelets and in the Weibel-Palade bodies of endothelial cells. Upon activation, P-selectin is rapidly translocated to the surface of endothelial cells and platelets to facilitate the “rolling” cell surface interaction with neutrophils and monocytes. Membrane-bound and soluble forms of P-selectin have been identified. Soluble P-selectin may be produced by shedding of membrane-bound P-selectin, either by proteolysis of the extracellular P-selectin molecule, or by proteolysis of components of the intracellular cytoskeleton in close proximity to the surface-bound P-selectin molecule (Fox, J. E., Blood Coagul. Fibrinolysis 5:291-304, 1994). Additionally, soluble P-selectin may be translated from mRNA that does not encode the N-terminal transmembrane domain (Dunlop, L. C. et al., J. Exp. Med. 175:1147-1150, 1992; Johnston, G. I. et al., J. Biol. Chem. 265:21381-21385, 1990).

Activated platelets can shed membrane-bound P-selectin and remain in the circulation, and the shedding of P-selectin can elevate the plasma P-selectin concentration by approximately 70 ng/ml (Michelson, A. D. et al., Proc. Natl. Acad. Sci. U.S.A. 93:11877-11882, 1996). Soluble P-selectin may also adopt a different conformation than membrane-bound P-selectin. Soluble P-selectin has a monomeric rod-like structure with a globular domain at one end, and the membrane-bound molecule forms rosette structures with the globular domain facing outward (Ushiyama, S. et al., J. Biol. Chem. 268:15229-15237, 1993). Soluble P-selectin may play an important role in regulating inflammation and thrombosis by blocking interactions between leukocytes and activated platelets and endothelial cells (Gamble, J. R. et al., Science 249:414-417, 1990). The normal plasma concentration of soluble P-selectin is <200 ng/ml. Blood is normally collected using citrate as an anticoagulant, but some studies have used EDTA plasma with additives such as prostaglandin E to prevent platelet activation. EDTA may be a suitable anticoagulant that will yield results comparable to those obtained using citrate. Furthermore, the plasma concentration of soluble P-selectin may not be affected by potential platelet activation during the sampling procedure. The plasma soluble P-selectin concentration was significantly elevated in patients with acute myocardial infarction and unstable angina, but not stable angina, even following an exercise stress test (Ikeda, H. et al., Circulation 92:1693-1696, 1995; Tomoda, H. and Aoki, N., Angiology 49:807-813, 1998; Hollander, J. E. et al., J. Am. Coll. Cardiol. 34:95-105, 1999; Kaikita, K. et al., Circulation 92:1726-1730, 1995; Ikeda, H. et al., Coron. Artery Dis. 5:515-518, 1994). The sensitivity and specificity of membrane-bound P-selectin versus soluble P-selectin for acute myocardial infarction is 71% versus 76% and 32% versus 45% (Hollander, J. E. et al., J. Am. Coll. Cardiol. 34:95-105, 1999). The sensitivity and specificity of membrane-bound P-selectin versus soluble P-selectin for unstable angina+acute myocardial infarction is 71% versus 79% and 30% versus 35% (Hollander, J. E. et al., J. Am. Coll. Cardiol. 34:95-105, 1999). P-selectin expression is greater in coronary atherectomy specimens from individuals with unstable angina than stable angina (Tenaglia, A. N. et al., Am. J. Cardiol. 79:742-747, 1997). Furthermore, plasma soluble P-selectin may be elevated to a greater degree in patients with acute myocardial infarction than in patients with unstable angina. Plasma soluble and membrane-bound P-selectin also is elevated in individuals with non-insulin dependent diabetes mellitus and congestive heart failure (Nomura, S. et al., Thromb. Haemost. 80:388-392, 1998; O'Connor, C. M. et al., Am. J. Cardiol. 83:1345-1349, 1999). Soluble P-selectin concentration is elevated in the plasma of individuals with idiopathic thrombocytopenic purpura, rheumatoid arthritis, hypercholesterolemia, acute stroke, atherosclerosis, hypertension, acute lung injury, connective tissue disease, thrombotic thrombocytopenic purpura, hemolytic uremic syndrome, disseminated intravascular coagulation, and chronic renal failure (Katayama, M. et al., Br. J. Haematol. 84:702-710, 1993; Haznedaroglu, I. C. et al., Acta Haematol. 101: 16-20, 1999; Ertenli, I. et al., J. Rheumatol. 25:1054-1058, 1998; Davi, G. et al., Circulation 97:953-957, 1998; Frijns, C. J. et al., Stroke 28:2214-2218, 1997; Blann, A. D. et al., Thromb. Haemost. 77:1077-1080, 1997; Blann, A. D. et al., J. Hum. Hypertens. 11:607-609, 1997; Sakamaki, F. et al., A. J. Respir. Crit. Care Med. 151:1821-1826, 1995; Takeda, I. et al., Int. Arch. Allergy Immunol. 105:128-134, 1994; Chong, B. H. et al., Blood 83:1535-1541, 1994; Bonomini, M. et al., Nephron 79:399-407, 1998). Additionally, any condition that involves platelet activation can potentially be a source of plasma elevations in P-selectin. P-selectin is rapidly presented on the cell surface following platelet of endothelial cell activation. Soluble P-selectin that has been translated from an alternative mRNA lacking a transmembrane domain is also released into the extracellular space following this activation. Soluble P-selectin can also be formed by proteolysis involving membrane-bound P-selectin, either directly or indirectly.

Plasma soluble P-selectin is elevated on admission in patients with acute myocardial infarction treated with tPA or coronary angioplasty, with a peak elevation occurring 4 hours after onset (Shimomura, H. et al., Am. J. Cardiol. 81:397-400, 1998). Plasma soluble P-selectin was elevated less than one hour following an anginal attack in patients with unstable angina, and the concentration decreased with time, approaching baseline more than 5 hours after attack onset (Ikeda, H. et al., Circulation 92:1693-1696, 1995). The plasma concentration of soluble P-selectin can approach 1 μg/ml in ACS (Ikeda, H. et al., Coron. Artery Dis. 5:515-518, 1994). Further investigation into the release of soluble P-selectin into and its removal from the bloodstream need to be conducted. P-selectin may be a sensitive and specific marker of platelet and endothelial cell activation, conditions that support thrombus formation and inflammation. It is not, however, a specific marker of ACS. When used with another marker that is specific for cardiac tissue injury, P-selectin may be useful in the discrimination of unstable angina and acute myocardial infarction from stable angina. Furthermore, soluble P-selectin may be elevated to a greater degree in acute myocardial infarction than in unstable angina. P-selectin normally exists in two forms, membrane-bound and soluble. Published investigations note that a soluble form of P-selectin is produced by platelets and endothelial cells, and by shedding of membrane-bound P-selectin, potentially through a proteolytic mechanism. Soluble P-selectin may prove to be the most useful currently identified marker of platelet activation, since its plasma concentration may not be as influenced by the blood sampling procedure as other markers of platelet activation, such as PF4 and β-TG.

Thrombin is a 37 kDa serine proteinase that proteolytically cleaves fibrinogen to form fibrin, which is ultimately integrated into a crosslinked network during clot formation. Antithrombin III (ATIII) is a 65 kDa serine proteinase inhibitor that is a physiological regulator of thrombin, factor XIa, factor XIIa, and factor IXa proteolytic activity. The inhibitory activity of ATIII is dependent upon the binding of heparin. Heparin enhances the inhibitory activity of ATIII by 2-3 orders of magnitude, resulting in almost instantaneous inactivation of proteinases inhibited by ATIII. ATIII inhibits its target proteinases through the formation of a covalent 1:1 stoichiometric complex. The normal plasma concentration of the approximately 100 kDa thrombin-ATIII complex (TAT) is <5 ng/ml (50 pM). TAT concentration is elevated in patients with acute myocardial infarction and unstable angina, especially during spontaneous ischemic episodes (Biasucci, L. M. et al., Am. J. Cardiol. 77:85-87, 1996; Kienast, J. et al., Thromb. Haemost. 70:550-553, 1993). Furthermore, TAT may be elevated in the plasma of individuals with stable angina (Manten, A. et al., Cardiovasc. Res. 40:389-395, 1998). Other published reports have found no significant differences in the concentration of TAT in the plasma of patients with ACS (Manten, A. et al., Cardiovasc. Res. 40:389-395, 1998; Hoffmeister, H. M. et al., Atherosclerosis 144:151-157, 1999). Further investigation is needed to determine plasma TAT concentration changes associated with ACS. Elevation of the plasma TAT concentration is associated with any condition associated with coagulation activation, including stroke, surgery, trauma, disseminated intravascular coagulation, and thrombotic thrombocytopenic purpura. TAT is formed immediately following thrombin activation in the presence of heparin, which is the limiting factor in this interaction. TAT has a half-life of approximately 5 minutes in the bloodstream (Biasucci, L. M. et al., Am. J. Cardiol. 77:85-87, 1996). TAT concentration is elevated in, exhibits a sharp drop after 15 minutes, and returns to baseline less than 1 hour following coagulation activation. The plasma concentration of TAT can approach 50 ng/ml in ACS (Biasucci, L. M. et al., Circulation 93:2121-2127, 1996). TAT is a specific marker of coagulation activation, specifically, thrombin activation.

von Willebrand factor (vWF) is a plasma protein produced by platelets, megakaryocytes, and endothelial cells composed of 220 kDa monomers that associate to form a series of high molecular weight multimers. These multimers normally range in molecular weight from 600-20,000 kDa. vWF participates in the coagulation process by stabilizing circulating coagulation factor VIII and by mediating platelet adhesion to exposed subendothelium, as well as to other platelets. The A1 domain of vWF binds to the platelet glycoprotein Ib-IX-V complex and non-fibrillar collagen type VI, and the A3 domain binds fibrillar collagen types I and III (Emsley, J. et al., J. Biol. Chem. 273:10396-10401, 1998). Other domains present in the vWF molecule include the integrin binding domain, which mediates platelet-platelet interactions, the protease cleavage domain, which appears to be relevant to the pathogenesis of type 11A von Willebrand disease. The interaction of vWF with platelets is tightly regulated to avoid interactions between vWF and platelets in normal physiologic conditions. vWF normally exists in a globular state, and it undergoes a conformation transition to an extended chain structure under conditions of high sheer stress, commonly found at sites of vascular injury. This conformational change exposes intramolecular domains of the molecule and allows vWF to interact with platelets. Furthermore, shear stress may cause vWF release from endothelial cells, making a larger number of vWF molecules available for interactions with platelets. The conformational change in vWF can be induced in vitro by the addition of non-physiological modulators like ristocetin and botrocetin (Miyata, S. et al., J. Biol. Chem. 271:9046-9053, 1996). At sites of vascular injury, vWF rapidly associates with collagen in the subendothelial matrix, and virtually irreversibly binds platelets, effectively forming a bridge between platelets and the vascular subendothelium at the site of injury. Evidence also suggests that a conformational change in vWF may not be required for its interaction with the subendothelial matrix (Sixma, J. J. and de Groot, P. G., Mayo Clin. Proc. 66:628-633, 1991). This suggests that vWF may bind to the exposed subendothelial matrix at sites of vascular injury, undergo a conformational change because of the high localized shear stress, and rapidly bind circulating platelets, which will be integrated into the newly formed thrombus.

Measurement of the total amount of vWF would allow one who is skilled in the art to identify changes in total vWF concentration. This measurement could be performed through the measurement of various forms of the vWF molecule. Measurement of the A1 domain would allow the measurement of active vWF in the circulation, indicating that a pro-coagulant state exists because the A1 domain is accessible for platelet binding. In this regard, an assay that specifically measures vWF molecules with both the exposed A1 domain and either the integrin binding domain or the A3 domain would also allow for the identification of active vWF that would be available for mediating platelet-platelet interactions or mediate crosslinking of platelets to vascular subendothelium, respectively. Measurement of any of these vWF forms, when used in an assay that employs antibodies specific for the protease cleavage domain may allow assays to be used to determine the circulating concentration of various vWF forms in any individual, regardless of the presence of von Willebrand disease. The normal plasma concentration of vWF is 5-10 μg/ml, or 60-110% activity, as measured by platelet aggregation. The measurement of specific forms of vWF may be of importance in any type of vascular disease, including stroke and cardiovascular disease. The plasma vWF concentration is reportedly elevated in individuals with acute myocardial infarction and unstable angina, but not stable angina (Goto, S. et al., Circulation 99:608-613, 1999; Tousoulis, D. et al., Int. J. Cardiol. 56:259-262, 1996; Yazdani, S. et al., J Am Coll Cardiol 30:1284-1287, 1997; Montalescot, G. et al., Circulation 98:294-299).

The plasma concentration of vWF may be elevated in conjunction with any event that is associated with endothelial cell damage or platelet activation. vWF is present at high concentration in the bloodstream, and it is released from platelets and endothelial cells upon activation. vWF would likely have the greatest utility as a marker of platelet activation or, specifically, conditions that favor platelet activation and adhesion to sites of vascular injury. The conformation of VWF is also known to be altered by high shear stress, as would be associated with a partially stenosed blood vessel. As the blood flows past a stenosed vessel, it is subjected to shear stress considerably higher than is encountered in the circulation of an undiseased individual.

Tissue factor (TF) is a 45 kDa cell surface protein expressed in brain, kidney, and heart, and in a transcriptionally regulated manner on perivascular cells and monocytes. TF forms a complex with factor VIIa in the presence of Ca²⁺ ions, and it is physiologically active when it is membrane bound. This complex proteolytically cleaves factor X to form factor Xa. It is normally sequestered from the bloodstream. Tissue factor can be detected in the bloodstream in a soluble form, bound to factor VIIa, or in a complex with factor VIIa, and tissue factor pathway inhibitor that can also include factor Xa. TF also is expressed on the surface of macrophages, which are commonly found in atherosclerotic plaques. The normal serum concentration of TF is <0.2 ng/ml (4.5 pM). The plasma TF concentration is elevated in patients with ischemic heart disease (Falciani, M. et al., Thromb. Haemost. 79:495-499, 1998). TF is elevated in patients with unstable angina and acute myocardial infarction, but not in patients with stable angina (Falciani, M. et al., Thromb. Haemost. 79:495-499, 1998; Suefuji, H. et al., Am. Heart J. 134:253-259, 1997; Misumi, K. et al., Am. J. Cardiol. 81:22-26, 1998). Furthermore, TF expression on macrophages and TF activity in atherosclerotic plaques is more common in unstable angina than stable angina (Soejima, H. et al., Circulation 99:2908-2913, 1999; Kaikita, K. et al., Arterioscler. Thromb. Vasc. Biol. 17:2232-2237, 1997; Ardissino, D. et al., Lancet 349:769-771, 1997).

The differences in plasma TF concentration in stable versus unstable angina may not be of statistical significance. Elevations in the serum concentration of TF are associated with any condition that causes or is a result of coagulation activation through the extrinsic pathway. These conditions can include subarachnoid hemorrhage, disseminated intravascular coagulation, renal failure, vasculitis, and sickle cell disease (Hirashima, Y. et al., Stroke 28:1666-1670, 1997; Takahashi, H. et al., Am. J. Hematol. 46:333-337, 1994; Koyama, T. et al., Br. J. Haematol. 87:343-347, 1994). TF is released immediately when vascular injury is coupled with extravascular cell injury. TF levels in ischemic heart disease patients can exceed 800 pg/ml within 2 days of onset (Falciani, M. et al., Thromb. Haemost. 79:495-499, 1998. TF levels were decreased in the chronic phase of acute myocardial infarction, as compared with the chronic phase (Suefuji, H. et al., Am. Heart J. 134:253-259, 1997). TF is a specific marker for activation of the extrinsic coagulation pathway and the presence of a general hypercoagulable state. It may be a sensitive marker of vascular injury resulting from plaque rupture

The coagulation cascade can be activated through either the extrinsic or intrinsic pathways. These enzymatic pathways share one final common pathway. The first step of the common pathway involves the proteolytic cleavage of prothrombin by the factor Xa/factor Va prothrombinase complex to yield active thrombin. Thrombin is a serine proteinase that proteolytically cleaves fibrinogen. Thrombin first removes fibrinopeptide A from fibrinogen, yielding desAA fibrin monomer, which can form complexes with all other fibrinogen-derived proteins, including fibrin degradation products, fibrinogen degradation products, desAA fibrin, and fibrinogen. The desAA fibrin monomer is generically referred to as soluble fibrin, as it is the first product of fibrinogen cleavage, but it is not yet crosslinked via factor XIIIa into an insoluble fibrin clot. DesAA fibrin monomer also can undergo further proteolytic cleavage by thrombin to remove fibrinopeptide B, yielding desAABB fibrin monomer. This monomer can polymerize with other desAABB fibrin monomers to form soluble desAABB fibrin polymer, also referred to as soluble fibrin or thrombus precursor protein (TpP^{TM}). TpP™ is the immediate precursor to insoluble fibrin, which forms a “mesh-like” structure to provide structural rigidity to the newly formed thrombus. In this regard, measurement of TpP™ in plasma is a direct measurement of active clot formation.

The normal plasma concentration of TpP™ is <6 ng/ml (Laurino, J. P. et al., Ann. Clin. Lab. Sci. 27:338-345, 1997). American Biogenetic Sciences has developed an assay for TpP™ (US Pat. Nos. 5,453,359 and 5,843,690) and states that its TpP™ assay can assist in the early diagnosis of acute myocardial infarction, the ruling out of acute myocardial infarction in chest pain patients, and the identification of patients with unstable angina that will progress to acute myocardial infarction. Other studies have confirmed that TpP™ is elevated in patients with acute myocardial infarction, most often within 6 hours of onset (Laurino, J. P. et al., Ann. Clin. Lab. Sci. 27:338-345, 1997; Carville, D. G. et al., Clin. Chem. 42:1537-1541, 1996). The plasma concentration of TpP™ is also elevated in patients with unstable angina, but these elevations may be indicative of the severity of angina and the eventual progression to acute myocardial infarction (Laurino, J. P. et al., Ann. Clin. Lab. Sci. 27:338-345, 1997). The concentration of TpP™ in plasma will theoretically be elevated during any condition that causes or is a result of coagulation activation, including disseminated intravascular coagulation, deep venous thrombosis, congestive heart failure, surgery, cancer, gastroenteritis, and cocaine overdose (Laurino, J. P. et al., Ann. Clin. Lab. Sci. 27:338-345, 1997). TpP™ is released into the bloodstream immediately following thrombin activation. TpP™ likely has a short half-life in the bloodstream because it will be rapidly converted to insoluble fibrin at the site of clot formation. Plasma TpP™ concentrations peak within 3 hours of acute myocardial infarction onset, returning to normal after 12 hours from onset. The plasma concentration of TpP™ can exceed 30 ng/ml in CVD (Laurino, J. P. et al., Ann. Clin. Lab. Sci. 27:338-345, 1997). TpP™ is a sensitive and specific marker of coagulation activation. It has been demonstrated that TpP™ is useful in the diagnosis of acute myocardial infarction, but only when it is used in conjunction with a specific marker of cardiac tissue injury.

(iii) Exemplary Markers Related to the Acute Phase Response

Human neutrophil elastase (HNE) is a 30 kDa serine proteinase that is normally contained within the azurophilic granules of neutrophils. HNE is released upon neutrophil activation, and its activity is regulated by circulating α₁-proteinase inhibitor. Activated neutrophils are commonly found in atherosclerotic plaques, and rupture of these plaques may result in the release of HNE. The plasma HNE concentration is usually measured by detecting HNE-α₁-PI complexes. The normal concentration of these complexes is 50 ng/ml, which indicates a normal concentration of approximately 25 ng/ml (0.8 nM) for HNE. HNE release also can be measured through the specific detection of fibrinopeptide Bβ₃₀₋₄₃, a specific HNE-derived fibrinopeptide, in plasma. Plasma HNE is elevated in patients with coronary stenosis, and its elevation is greater in patients with complex plaques than those with simple plaques (Kosar, F. et al., Angiology 49:193-201, 1998; Amaro, A. et al., Eur. Heart J. 16:615-622, 1995). Plasma HNE is not significantly elevated in patients with stable angina, but is elevated inpatients with unstable angina and acute myocardial infarction, as determined by measuring fibrinopeptide Bβ₃₀₋₄₃, with concentrations in unstable angina being 2.5-fold higher than those associated with acute myocardial infarction (Dinerman, J. L. et al., J. Am. Coll. Cardiol. 15:1559-1563, 1990; Mehta, J. et al., Circulation 79:549-556, 1989). Serum HNE is elevated in cardiac surgery, exercise-induced muscle damage, giant cell arteritis, acute respiratory distress syndrome, appendicitis, pancreatitis, sepsis, smoking-associated emphysema, and cystic fibrosis (Genereau, T. et al., J. Rheumatol. 25:710-713, 1998; Mooser, V. et al., Arterioscler. Thromb. Vasc. Biol. 19:1060-1065, 1999; Gleeson, M. et al. Eur. J. Appl. Physiol. 77:543-546, 1998; Gando, S. et al., J. Trauma 42:1068-1072, 1997; Eriksson, S. et al., Eur. J. Surg. 161:901-905, 1995; Liras, G. et al., Rev. Esp. Enferm. Dig. 87:641-652, 1995; Endo, S. et al., J. Inflamm. 45:136-142, 1995; Janoff, A., Annu Rev Med 36:207-216, 1985). HNE may also be released during blood coagulation (Plow, E. F. and Plescia, J., Thromb. Haemost. 59:360-363, 1988; Plow, E. F., J. Clin. Invest. 69:564-572, 1982). Serum elevations of HNE could also be associated with any non-specific infection or inflammatory state that involves neutrophil recruitment and activation. It is most likely released upon plaque rupture, since activated neutrophils are present in atherosclerotic plaques. HNE is presumably cleared by the liver after it has formed a complex with α₁-PI.

Inducible nitric oxide synthase (iNOS) is a 130 kDa cytosolic protein in epithelial cells macrophages whose expression is regulated by cytokines, including interferon-γ, interleukin-1β, interleukin-6, and tumor necrosis factor α, and lipopolysaccharide. iNOS catalyzes the synthesis of nitric oxide (NO) from L-arginine, and its induction results in a sustained high-output production of NO, which has antimicrobial activity and is a mediator of a variety of physiological and inflammatory events. NO production by iNOS is approximately 100 fold more than the amount produced by constitutively-expressed NOS (Depre, C. et al., Cardiovasc. Res. 41:465-472, 1999). There are no published investigations of plasma iNOS concentration changes associated with ACS. iNOS is expressed in coronary atherosclerotic plaque, and it may interfere with plaque stability through the production of peroxynitrate, which is a product of NO and superoxide and enhances platelet adhesion and aggregation (Depre, C. et al., Cardiovasc. Res. 41:465-472, 1999). iNOS expression during myocardial ischemia may not be elevated, suggesting that iNOS may be useful in the differentiation of angina from acute myocardial infarction (Hammerman, S. I. et al., Am. J. Physiol. 277:H1579-H1592, 1999; Kaye, D. M. et al., Life Sci 62:883-887, 1998). Elevations in the plasma iNOS concentration may be associated with cirrhosis, iron-deficiency anemia, or any other condition that results in macrophage activation, including bacterial infection (Jimenez, W. et al., Hepatology 30:670-676, 1999; Ni, Z. et al., Kidney Int. 52:195-201, 1997). iNOS may be released into the bloodstream as a result of atherosclerotic plaque rupture, and the presence of increased amounts of iNOS in the bloodstream may not only indicate that plaque rupture has occurred, but also that an ideal environment has been created to promote platelet adhesion. However, iNOS is not specific for atherosclerotic plaque rupture, and its expression can be induced during non-specific inflammatory conditions.

Lysophosphatidic acid (LPA) is a lysophospholipid intermediate formed in the synthesis of phosphoglycerides and triacylglycerols. It is formed by the acylation of glycerol-3 phosphate by acyl-coenzyme A and during mild oxidation of low-density lipoprotein (LDL). LPA is a lipid second messenger with vasoactive properties, and it can function as a platelet activator. LPA is a component of atherosclerotic lesions, particularly in the core, which is most prone to rupture (Siess, W., Proc. Natl. Acad. Sci. U.S.A. 96, 6931-6936, 1999). The normal plasma LPA concentration is 540 nM. Serum LPA is elevated in renal failure and in ovarian cancer and other gynecologic cancers (Sasagawa, T. et al., J. Nutr. Sci. Vitaminol. (Tokyo) 44:809-818, 1998; Xu, Y. et al., JAMA 280:719-723, 1998). In the context of unstable angina, LPA is most likely released as a direct result of plaque rupture. The plasma LPA concentration can exceed 60 μM in patients with gynecologic cancers (Xu, Y. et al., JAMA 280:719-723, 1998). Serum LPA may be a useful marker of atherosclerotic plaque rupture.

Malondialdehyde-modified low-density lipoprotein (MDA-modified LDL) is formed during the oxidation of the apoB-100 moiety of LDL as a result of phospholipase activity, prostaglandin synthesis, or platelet activation. MDA-modified LDL can be distinguished from oxidized LDL because MDA modifications of LDL occur in the absence of lipid peroxidation (Holvoet, P., Acta Cardiol. 53:253-260, 1998). The normal plasma concentration of MDA-modified LDL is less than 4 μg/ml (˜10 μM). Plasma concentrations of oxidized LDL are elevated in stable angina, unstable angina, and acute myocardial infarction, indicating that it may be a marker of atherosclerosis (Holvoet, P., Acta Cardiol. 53:253-260, 1998; Holvoet, P. et al., Circulation 98:1487-1494, 1998). Plasma MDA-modified LDL is not elevated in stable angina, but is significantly elevated in unstable angina and acute myocardial infarction (Holvoet, P., Acta Cardiol. 53:253-260, 1998; Holvoet, P. et al., Circulation 98:1487-1494, 1998; Holvoet, P. et al., JAMA 281:1718-1721, 1999). Plasma MDA-modified LDL is elevated in individuals with beta-thallasemia and in renal transplant patients (Livrea, M. A. et al., Blood 92:3936-3942, 1998; Ghanem, H. et al., Kidney Int. 49:488-493, 1996; van den Dorpel, M. A. et al., Transpl. Int. 9 Suppl. 1:S54-S57, 1996). Furthermore, serum MDA-modified LDL may be elevated during hypoxia (Balagopalakrishna, C. et al., Adv. Exp. Med. Biol. 411:337-345, 1997). The plasma concentration of MDA-modified LDL is elevated within 6-8 hours from the onset of chest pain. Plasma concentrations of MDA-modified LDL can approach 20 μg/ml (˜50 μM) in patients with acute myocardial infarction, and 15 μg/ml (˜40 μM) in patients with unstable angina (Holvoet, P. et al., Circulation 98:1487-1494, 1998). Plasma MDA-modified LDL has a half-life of less than 5 minutes in mice (Ling, W. et al., J. Clin. Invest. 100:244-252, 1997). MDA-modified LDL appears to be a specific marker of atherosclerotic plaque rupture in acute coronary symptoms. It is unclear, however, if elevations in the plasma concentration of MDA-modified LDL are a result of plaque rupture or platelet activation. The most reasonable explanation is that the presence of increased amounts of MDA-modified LDL is an indication of both events. MDA-modified LDL may be useful in discriminating unstable angina and acute myocardial infarction from stable angina.

Matrix metalloproteinase-1 (MMP-1), also called collagenase-1, is a 41/44 kDa zinc- and calcium-binding proteinase that cleaves primarily type I collagen, but can also cleave collagen types II, III, VII and X. The active 41/44 kDa enzyme can undergo autolysis to the still active 22/27 kDa form. MMP-1 is synthesized by a variety of cells, including smooth muscle cells, mast cells, macrophage-derived foam cells, T lymphocytes, and endothelial cells (Johnson, J. L. et al., Arterioscler. Thromb. Vasc. Biol. 18:1707-1715, 1998). MMP-1, like other MMPs, is involved in extracellular matrix remodeling, which can occur following injury or during intervascular cell migration. MMP-1 can be found in the bloodstream either in a free form or in complex with TIMP-1, its natural inhibitor. MMP-1 is normally found at a concentration of <25 ng/ml in plasma. MMP-1 is found in the shoulder region of atherosclerotic plaques, which is the region most prone to rupture, and may be involved in atherosclerotic plaque destabilization (Johnson, J. L. et al., Arterioscler. Thromb. Vasc. Biol. 18:1707-1715, 1998). Furthermore, MMP-1 has been implicated in the pathogenesis of myocardial reperfusion injury (Shibata, M. et al., Angiology 50:573-582, 1999). Serum MMP-1 may be elevated inflammatory conditions that induce mast cell degranulation. Serum MMP-1 concentrations are elevated in patients with arthritis and systemic lupus erythematosus (Keyszer, G. et al., Z Rheumatol 57:392-398, 1998; Keyszer, G. J. Rheumatol. 26:251-258, 1999). Serum MMP-1 also is elevated in patients with prostate cancer, and the degree of elevation corresponds to the metastatic potential of the tumor (Baker, T. et al., Br. J. Cancer 70:506-512, 1994). The serum concentration of MMP-1 may also be elevated in patients with other types of cancer. Serum MMP-1 is decreased in patients with hemochromatosis and also in patients with chronic viral hepatitis, where the concentration is inversely related to the severity (George, D. K. et al., Gut 42:715-720, 1998; Murawaki, Y. et al., J. Gastroenterol. Hepatol. 14:138-145, 1999). Serum MMP-1 was decreased in the first four days following acute myocardial infarction, and increased thereafter, reaching peak levels 2 weeks after the onset of acute myocardial infarction (George, D. K. et al., Gut 42:715-720, 1998).

Matrix metalloproteinase-2 (MMP-2), also called gelatinase A, is a 66 kDa zinc- and calcium-binding proteinase that is synthesized as an inactive 72 kDa precursor. Mature MMP-3 cleaves type I gelatin and collagen of types IV, V, VII, and X. MMP-2 is synthesized by a variety of cells, including vascular smooth muscle cells, mast cells, macrophage-derived foam cells, T lymphocytes, and endothelial cells (Johnson, J. L. et al., Arterioscler. Thromb. Vasc. Biol. 18:1707-1715, 1998). MMP-2 is usually found in plasma in complex with TIMP-2, its physiological regulator (Murawaki, Y. et al., J. Hepatol. 30:1090-1098, 1999). The normal plasma concentration of MMP-2 is <˜550 ng/ml (8 nM). MMP-2 expression is elevated in vascular smooth muscle cells within atherosclerotic lesions, and it may be released into the bloodstream in cases of plaque instability (Kai, H. et al., J. Am. Coll. Cardiol. 32:368-372, 1998). Furthermore, MMP-2 has been implicated as a contributor to plaque instability and rupture (Shah, P. K. et al., Circulation 92:1565-1569, 1995). Serum MMP-2 concentrations were elevated in patients with stable angina, unstable angina, and acute myocardial infarction, with elevations being significantly greater in unstable angina and acute myocardial infarction than in stable angina (Kai, H. et al., J. Am. Coll. Cardiol. 32:368-372, 1998). There was no change in the serum MMP-2 concentration in individuals with stable angina following a treadmill exercise test (Kai, H. et al., J. Am. Coll. Cardiol. 32:368-372, 1998). Serum and plasma MMP-2 is elevated in patients with gastric cancer, hepatocellular carcinoma, liver cirrhosis, urothelial carcinoma, rheumatoid arthritis, and lung cancer (Murawaki, Y. et al., J. Hepatol. 30:1090-1098, 1999; Endo, K. et al., Anticancer Res. 17:2253-2258, 1997; Gohji, K. et al., Cancer 78:2379-2387, 1996; Gruber, B. L. et al., Clin. Immunol. Immunopathol. 78:161-171, 1996; Garbisa, S. et al., Cancer Res. 52:4548-4549, 1992). Furthermore, MMP-2 may also be translocated from the platelet cytosol to the extracellular space during platelet aggregation (Sawicki, G. et al., Thromb. Haemost. 80:836-839, 1998). MMP-2 was elevated on admission in the serum of individuals with unstable angina and acute myocardial infarction, with maximum levels approaching 1.5 μg/ml (25 nM) (Kai, H. et al., J. Am. Coll. Cardiol. 32:368-372, 1998). The serum MMP-2 concentration peaked 1-3 days after onset in both unstable angina and acute myocardial infarction, and started to return to normal after 1 week (Kai, H. et al., J. Am. Coll. Cardiol. 32:368-372, 1998).

Matrix metalloproteinase-3 (MMP-3), also called stromelysin-1, is a 45 kDa zinc- and calcium-binding proteinase that is synthesized as an inactive 60 kDa precursor. Mature MMP-3 cleaves proteoglycan, fibrinectin, laminin, and type IV collagen, but not type I collagen. MMP-3 is synthesized by a variety of cells, including smooth muscle cells, mast cells, macrophage-derived foam cells, T lymphocytes, and endothelial cells (Johnson, J. L. et al., Arterioscler. Thromb. Vasc. Biol. 18:1707-1715, 1998). MMP-3, like other MMPs, is involved in extracellular matrix remodeling, which can occur following injury or during intervascular cell migration. MMP-3 is normally found at a concentration of <125 ng/ml in plasma. The serum MMP-3 concentration also has been shown to increase with age, and the concentration in males is approximately 2 times higher in males than in females (Manicourt, D. H. et al., Arthritis Rheum. 37:1774-1783, 1994). MMP-3 is found in the shoulder region of atherosclerotic plaques, which is the region most prone to rupture, and may be involved in atherosclerotic plaque destabilization (Johnson, J. L. et al., Arterioscler. Thromb. Vasc. Biol. 18:1707-1715, 1998). Therefore, MMP-3 concentration may be elevated as a result of atherosclerotic plaque rupture in unstable angina. Serum MMP-3 may be elevated inflammatory conditions that induce mast cell degranulation. Serum MMP-3 concentrations are elevated in patients with arthritis and systemic lupus erythematosus (Zucker, S. et al. J. Rheumatol. 26:78-80, 1999; Keyszer, G. et al., Z Rheumatol. 57:392-398, 1998; Keyszer, G. et al. J. Rheumatol. 26:251-258, 1999). Serum MMP-3 also is elevated in patients with prostate and urothelial cancer, and also glomerulonephritis (Lein, M. et al., Urologe A 37:377-381, 1998; Gohji, K. et al., Cancer 78:2379-2387, 1996; Akiyama, K. et al., Res. Commun. Mol. Pathol. Pharmacol. 95:115-128, 1997). The serum concentration of MMP-3 may also be elevated in patients with other types of cancer. Serum MMP-3 is decreased in patients with hemochromatosis (George, D. K. et al., Gut 42:715-720, 1998).

Matrix metalloproteinase-9 (MMP-9) also called gelatinase B, is an 84 kDa zinc- and calcium-binding proteinase that is synthesized as an inactive 92 kDa precursor. Mature MMP-9 cleaves gelatin types I and V, and collagen types IV and V. MMP-9 exists as a monomer, a homodimer, and a heterodimer with a 25 kDa α₂-microglobulin-related protein (Triebel, S. et al., FEBS Lett. 314:386-388, 1992). MMP-9 is synthesized by a variety of cell types, most notably by neutrophils. The normal plasma concentration of MMP-9 is <35 ng/ml (400 pM). MMP-9 expression is elevated in vascular smooth muscle cells within atherosclerotic lesions, and it may be released into the bloodstream in cases of plaque instability (Kai, H. et al., J. Am. Coll. Cardiol. 32:368-372, 1998). Furthermore, MMP-9 may have a pathogenic role in the development of ACS (Brown, D. L. et al., Circulation 91:2125-2131, 1995). Plasma MMP-9 concentrations are significantly elevated in patients with unstable angina and acute myocardial infarction, but not stable angina (Kai, H. et al., J. Am. Coll. Cardiol. 32:368-372, 1998). The elevations in patients with acute myocardial infarction may also indicate that those individuals were suffering from unstable angina. Elevations in the plasma concentration of MMP-9 may also be greater in unstable angina than in acute myocardial infarction. There was no significant change in plasma MMP-9 levels after a treadmill exercise test in patients with stable angina (Kai, H. et al., J. Am. Coll. Cardiol. 32:368-372, 1998). Plasma MMP-9 is elevated in individuals with rheumatoid arthritis, septic shock, giant cell arteritis and various carcinomas (Gruber, B. L. et al., Clin. Immunol. Immunopathol. 78:161-171, 1996; Nakamura, T. et al., Am. J. Med. Sci. 316:355-360, 1998; Blankaert, D. et al., J. Acquir. Immune Defic. Syndr. Hum. Retrovirol. 18:203-209, 1998; Endo, K. et al. Anticancer Res. 17:2253-2258, 1997; Hayasaka, A. et al., Hepatology 24:1058-1062, 1996; Moore, D. H. et al., Gynecol. Oncol. 65:78-82, 1997; Sorbi, D. et al., Arthritis Rheum. 39:1747-1753, 1996; lizasa, T. et al., Clin., Cancer Res. 5:149-153, 1999). Furthermore, the plasma MMP-9 concentration may be elevated in stroke and cerebral hemorrhage (Mun-Bryce, S. and Rosenberg, G. A., J. Cereb. Blood Flow Metab. 18:1163-1172, 1998; Romanic, A. M. et al., Stroke 29:1020-1030, 1998; Rosenberg, G. A., J. Neurotrauma 12:833-842, 1995). MMP-9 was elevated on admission in the serum of individuals with unstable angina and acute myocardial infarction, with maximum levels approaching 150 ng/ml (1.7 nM) (Kai, H. et al., J. Am. Coll. Cardiol. 32:368-372, 1998). The serum MMP-9 concentration was highest on admission in patients unstable angina, and the concentration decreased gradually after treatment, approaching baseline more than 1 week after onset (Kai, H. et al., J. Am. Coll. Cardiol. 32:368-372, 1998).

The balance between matrix metalloproteinases and their inhibitors is a critical factor which affects tumor invasion and metastasis. The TIMP family represents a class of small (21-28 kDa) related proteins that inhibit the metalloproteinases. Tissue inhibitor of metalloproteinase 1 (TIMP 1) is reportedly involved in the regulation of bone modeling and remodeling in normal developing human bone, involved in the invasive phenotype of acute myelogenous leukemia, demonstrating polymorphic X-chromosome inactivation. TIMP 1 is known to act on mmp-1, mmp-2, mmp-3, mmp-7, mmp-8, mmp-9, mmp-10, mmp-11, mmp-12, mmp-13 and mmp-16. Tissue inhibitor of metalloproteinase 2 (TIMP2) complexes with metalloproteinases (such as collagenases) and irreversibly inactivates them. TIMP 2 is known to act on mmp-1, mmp-2, mmp-3, mmp-7, mmp-8, mmp-9, mmp-10, mmp-13, mmp-14, mmp-15, mmp-16 and mmp-19. Two alternatively spliced forms may be associated with SYN4, and involved in the invasive phenotype of acute myelogenous leukemia. Unlike the inducible expression of some other TIMP gene family members, the expression of this gene is largely constitutive. Tissue inhibitor of metalloproteinase 3 (TIMP3) antagonizes matrix metalloproteinase activity and can suppress tumor growth, angiogenesis, invasion, and metastasis. Loss of TIMP-3 has been related to the acquisition of tumorigenesis.

The inter-alpha-inhibitor (I-α-I) family encompasses four plasma proteins (free bikunin, I-α-I (or inter-α-trypsin inhibitor), pre-alpha-inhibitor (P-α-I) and inter-α-like inhibitor (I-α-LI). Each of the last three proteins is a distinct assembly of one bikunin chain with one or more unique heavy (H) chains designated H1, H2 and H3. The three H chains and the bikunin chain are encoded by four distinct mRNAs. These molecules and chains, as well as the corresponding mRNAs, have been quantified in sera from patients with or without mild or severe acute infection. In acute inflammation the H2 and bikunin chains are reported to be down-regulated and the relevant molecules (I-α-I and I-α-LI) behave as negative acute-phase proteins, whereas the H3 chain is up-regulated and the corresponding P-α-I molecule is a positive acute-phase protein. The H1 gene does not seem to be affected by the inflammatory condition. See, e.g., Salier et al., Biochem. J. 315: 1-9, 1996; see also, International Publication No. WO01/63280.

(iv) Exemplary Markers Related to Inflammation

Pulmonary surfactant protein D (SP-D) is a 43 kDa protein synthesized and secreted into the airspaces of the lung by the respiratory epithelium. At the alveolar level, SP-D is constitutively synthesized and secreted by alveolar type II cells. SP-D, a collagenous calcium-dependent lectin (or collectin), binds to surface glycoconjugates expressed by a wide variety of microorganisms, and to oligosaccharides associated with the surface of various complex organic antigens. SP-D also specifically interacts with glycoconjugates and other molecules expressed on the surface of macrophages, neutrophils, and lymphocytes. In addition, SP-D binds to specific surfactant-associated lipids and can influence the organization of lipid mixtures containing phosphatidylinositol in vitro. Consistent with these diverse in vitro activities is the observation that SP-D-deficient transgenic mice show abnormal accumulations of surfactant lipids, and respond abnormally to challenge with respiratory viruses and bacterial lipopolysaccharides. The phenotype of macrophages isolated from the lungs of SP-D-deficient mice is altered, and there is circumstantial evidence that abnormal oxidant metabolism and/or increased metalloproteinase expression contributes to the development of emphysema. The expression of SP-D is increased in response to many forms of lung injury, and deficient accumulation of appropriately oligomerized SP-D might contribute to the pathogenesis of a variety of human lung diseases. See, e.g., Crouch, Respir. Res. 1: 93-108 (2000).

Interleukins (ILs) are part of a larger class of polypeptides known as cytokines. These are messenger molecules that transmit signals between various cells of the immune system. They are mostly secreted by macrophages and lymphocytes and their production is induced in response to injury or infection. Their actions influence other cells of the immune system as well as other tissues and organs including the liver and brain. There are at least 18 ILs described. IL-1β, IL-2, IL-4, IL-6, IL-8, IL-10, IL-12, IL-13, IL-18, IL-22, IL-23, and IL-25 are preferred for use as markers in the present invention. The following table shows selected functions of representative interleukins.

TABLE 1
Selected Functions of Representative Interleukins*

| Functions | IL-1 | IL-2 | IL-4 | IL-6 | IL-8 | IL-10 |
|---|---|---|---|---|---|---|
| Enhance immune responses | + | + | + | + | − | + |
| Suppress immune responses | − | − | − | − | − | + |
| Enhance inflammation | + | + | + | + | + | − |
| Suppress inflammation | − | − | − | − | − | + |
| Promote cell growth | + | + | − | − | − | − |
| Chemotactic (chemokines) | − | − | − | − | + | − |
| Pyrogenic | + | − | − | − | − | − |

Interleukin-1β (IL-1β) is a 17 kDa secreted proinflammatory cytokine that is involved in the acute phase response and is a pathogenic mediator of many diseases. IL-1β is normally produced by macrophages and epithelial cells. IL-1β is also released from cells undergoing apoptosis. The normal serum concentration of IL-1β is <30 pg/ml (1.8 pM). In theory, IL-1β would be elevated earlier than other acute phase proteins such as CRP in unstable angina and acute myocardial infarction, since IL-1β is an early participant in the acute phase response. Furthermore, IL-1β is released from cells undergoing apoptosis, which may be activated in the early stages of ischemia. In this regard, elevation of the plasma IL-1β concentration associated with ACS requires further investigation using a high-sensitivity assay. Elevations of the plasma IL-1β concentration are associated with activation of the acute phase response in proinflammatory conditions such as trauma and infection. IL-1β has a biphasic physiological half-life of 5 minutes followed by 4 hours (Kudo, S. et al., Cancer Res. 50:5751-5755, 1990). IL-1β is released into the extracellular milieu upon activation of the inflammatory response or apoptosis.

Interleukin-1 receptor antagonist (IL-1ra) is a 17 kDa member of the IL-1 family predominantly expressed in hepatocytes, epithelial cells, monocytes, macrophages, and neutrophils. IL-1ra has both intracellular and extracellular forms produced through alternative splicing. IL-1ra is thought to participate in the regulation of physiological IL-1 activity. IL-1ra has no IL-1-like physiological activity, but is able to bind the IL-1 receptor on T-cells and fibroblasts with an affinity similar to that of IL-1β blocking the binding of IL-1a and IL-1β and inhibiting their bioactivity (Stockman, B. J. et al., Biochemistry 31:5237-5245, 1992; Eisenberg, S. P. et al., Proc. Natl. Acad. Sci. U.S.A. 88:5232-5236, 1991; Carter, D. B. et al., Nature 344:633-638, 1990). IL-1ra is normally present in higher concentrations than IL-1 in plasma, and it has been suggested that IL-1ra levels are a better correlate of disease severity than IL-1 (Biasucci, L. M. et al., Circulation 99:2079-2084, 1999). Furthermore, there is evidence that IL-1ra is an acute phase protein (Gabay, C. et al., J. Clin. Invest. 99:2930-2940, 1997). The normal plasma concentration of IL-1ra is <200 pg/ml (12 pM). The plasma concentration of IL-1ra is elevated in patients with acute myocardial infarction and unstable angina that proceeded to acute myocardial infarction, death, or refractory angina (Biasucci, L. M. et al., Circulation 99:2079-2084, 1999; Latini, R. et al., J. Cardiovasc. Pharmacol. 23:1-6, 1994). Furthermore, IL-1ra was significantly elevated in severe acute myocardial infarction as compared to uncomplicated acute myocardial infarction (Latini, R. et al., J. Cardiovasc. Pharmacol. 23:1-6, 1994). Elevations in the plasma concentration of IL-1ra are associated with any condition that involves activation of the inflammatory or acute phase response, including infection, trauma, and arthritis. IL-1ra is released into the bloodstream in pro-inflammatory conditions, and it may also be released as a participant in the acute phase response. The major sources of clearance of IL-1ra from the bloodstream appear to be kidney and liver (Kim, D. C. et al., J. Pharm. Sci. 84:575-580, 1995). IL-1ra concentrations were elevated in the plasma of individuals with unstable angina within 24 hours of onset, and these elevations may even be evident within 2 hours of onset (Biasucci, L. M. et al., Circulation 99:2079-2084, 1999). In patients with severe progression of unstable angina, the plasma concentration of IL-1ra was higher 48 hours after onset than levels at admission, while the concentration decreased in patients with uneventful progression (Biasucci, L. M. et al., Circulation 99:2079-2084, 1999). In addition, the plasma concentration of IL-1ra associated with unstable angina can approach 1.4 ng/ml (80 pM). Changes in the plasma concentration of IL-1ra appear to be related to disease severity. Furthermore, it is likely released in conjunction with or soon after IL-1 release in pro-inflammatory conditions, and it is found at higher concentrations than IL-1. This indicates that IL-1ra may be a useful indirect marker of IL-1 activity, which elicits the production of IL-6.

Interleukin-6 (IL-6) is a 20 kDa secreted protein that is a hematopoietin family proinflammatory cytokine. IL-6 is an acute-phase reactant and stimulates the synthesis of a variety of proteins, including adhesion molecules. Its major function is to mediate the acute phase production of hepatic proteins, and its synthesis is induced by the cytokine IL-1. IL-6 is normally produced by macrophages and T lymphocytes. The normal serum concentration of IL-6 is <3 pg/ml (0.15 pM). The plasma concentration of IL-6 is elevated in patients with acute myocardial infarction and unstable angina, to a greater degree in acute myocardial infarction (Biasucci, L. M. et al., Circulation 94:874-877, 1996; Manten, A. et al., Cardiovasc. Res. 40:389-395, 1998; Biasucci, L. M. et al., Circulation 99:2079-2084, 1999). IL-6 is not significantly elevated in the plasma of patients with stable angina (Biasucci, L. M. et al., Circulation 94:874-877, 1996; Manten, A. et al., Cardiovasc. Res. 40:389-395, 1998). Furthermore, IL-6 concentrations increase over 48 hours from onset in the plasma of patients with unstable angina with severe progression, but decrease in those with uneventful progression (Biasucci, L. M. et al., Circulation 99:2079-2084, 1999). This indicates that IL-6 may be a useful indicator of disease progression. Plasma elevations of IL-6 are associated with any nonspecific proinflammatory condition such as trauma, infection, or other diseases that elicit an acute phase response. IL-6 has a half-life of 4.2 hours in the bloodstream and is elevated following acute myocardial infarction and unstable angina (Manten, A. et al., Cardiovasc. Res. 40:389-395, 1998). The plasma concentration of IL-6 is elevated within 8-12 hours of acute myocardial infarction onset, and can approach 100 pg/ml. The plasma concentration of IL-6 in patients with unstable angina was elevated at peak levels 72 hours after onset, possibly due to the severity of insult (Biasucci, L. M. et al., Circulation 94:874-877, 1996).

Interleukin-8 (IL-8) is a 6.5 kDa chemokine produced by monocytes, endothelial cells, alveolar macrophages and fibroblasts. IL-8 induces chemotaxis and activation of neutrophils and T cells. Known IL-8-related molecules include IL-8₆₋₇₇, IL-8_{7-77,} IL-8₈₋₇₇, and IL-8₉₋₇₇.

Interleukin 10 (“IL-10”) is a 160 amino acid (18.5 kDa predicted mass) cytokine that is a member of the four α-helix bundle family of cytokines. In solution, IL-10 forms a homodimer having an apparent molecular weight of 39 kDa. The human IL-10 gene is located on chromosome 1. Viera et al., Proc. Natl. Acad Sci. USA 88: 1172-76 (1991); Kim et al., J. Immunol. 148: 3618-23 (1992). Overproduction of IL-10 has been identified as a marker in sepsis, and is predictive of severity and mortality. Gogos et al., J. Infect. Dis. 181: 176-80 (2000).

In addition to the interleukins, there exist numerous molecules of the “small inducible cytokine” family, such as CXCL6 (human: Swiss-Prot P80162), CXCL13 (human: Swiss-Prot O43927), CXCL16 (human: Swiss-Prot Q9H2A7), CCL8 (human: Swiss-Prot P80075), CCL20 (human: Swiss-Prot P78556), CCL23 (human: Swiss-ProtP55773), and CCL26 (human: Swiss-Prot Q9Y258).

Tumor necrosis factor α (TNFα) is a 17 kDa secreted proinflammatory cytokine that is involved in the acute phase response and is a pathogenic mediator of many diseases. TNFα is normally produced by macrophages and natural killer cells. TNF-alpha is a protein of 185 amino acids glycosylated at positions 73 and 172. It is synthesized as a precursor protein of 212 amino acids. Monocytes express at least five different molecular forms of TNF-alpha with molecular masses of 21.5-28 kDa. They mainly differ by post-translational alterations such as glycosylation and phosphorylation. The normal serum concentration of TNFα is <40 pg/ml (2 pM). The plasma concentration of TNFα is elevated in patients with acute myocardial infarction, and is marginally elevated in patients with unstable angina (Li, D. et al., Am. Heart J. 137:1145-1152, 1999; Squadrito, F. et al., Inflamm. Res. 45:14-19, 1996; Latini, R. et al., J. Cardiovasc. Pharmacol. 23:1-6, 1994; Carlstedt, F. et al., J. Intern. Med. 242:361-365, 1997). Elevations in the plasma concentration of TNFα are associated with any proinflammatory condition, including trauma, stroke, and infection. TNFα has a half-life of approximately 1 hour in the bloodstream, indicating that it may be removed from the circulation soon after symptom onset. In patients with acute myocardial infarction, TNFα was elevated 4 hours after the onset of chest pain, and gradually declined to normal levels within 48 hours of onset (Li, D. et al., Am. Heart J. 137:1145-1152, 1999). The concentration of TNFα in the plasma of acute myocardial infarction patients exceeded 300 pg/ml (15 pM) (Squadrito, F. et al., Inflamm. Res. 45:14-19, 1996). Release of TNFα by monocytes has also been related to the progression of pneumoconiosis in coal workers. Schins and Borm, Occup. Environ. Med. 52: 441-50 (1995).

Soluble intercellular adhesion molecule (sICAM-1), also called CD54, is a 85-110 kDa cell surface-bound immunoglobulin-like integrin ligand that facilitates binding of leukocytes to antigen-presenting cells and endothelial cells during leukocyte recruitment and migration. sICAM-1 is normally produced by vascular endothelium, hematopoietic stem cells and non-hematopoietic stem cells, which can be found in intestine and epidermis. sICAM-1 can be released from the cell surface during cell death or as a result of proteolytic activity. The normal plasma concentration of sICAM-1 is approximately 250 ng/ml (2.9 nM). The plasma concentration of sICAM-1 is significantly elevated in patients with acute myocardial infarction and unstable angina, but not stable angina (Pellegatta, F. et al., J. Cardiovasc. Pharmacol. 30:455-460, 1997; Miwa, K. et al., Cardiovasc. Res. 36:37-44, 1997; Ghaisas, N. K. et al., Am. J. Cardiol. 80:617-619, 1997; Ogawa, H. et al., Am. J. Cardiol. 83:38-42, 1999). Furthermore, ICAM-1 is expressed in atherosclerotic lesions and in areas predisposed to lesion formation, so it may be released into the bloodstream upon plaque rupture (Iiyama, K. et al., Circ. Res. 85:199-207, 1999; Tenaglia, A. N. et al., Am. J. Cardiol. 79:742-747, 1997). Elevations of the plasma concentration of sICAM-1 are associated with ischemic stroke, head trauma, atherosclerosis, cancer, preeclampsia, multiple sclerosis, cystic fibrosis, and other nonspecific inflammatory states (Kim, J. S., J. Neurol. Sci. 137:69-78, 1996; Laskowitz, D. T. et al., J. Stroke Cerebrovasc. Dis. 7:234-241, 1998). The plasma concentration of sICAM-1 is elevated during the acute stage of acute myocardial infarction and unstable angina. The elevation of plasma sICAM-1 reaches its peak within 9-12 hours of acute myocardial infarction onset, and returns to normal levels within 24 hours (Pellegatta, F. et al., J. Cardiovasc. Pharmacol. 30:455-460, 1997). The plasma concentration of sICAM can approach 700 ng/ml (8 nM) in patients with acute myocardial infarction (Pellegatta, F. et al., J. Cardiovasc. Pharmacol. 30:455-460, 1997). sICAM-1 is elevated in the plasma of individuals with acute myocardial infarction and unstable angina, but it is not specific for these diseases. It may, however, be useful marker in the differentiation of acute myocardial infarction and unstable angina from stable angina since plasma elevations are not associated with stable angina. Interestingly, ICAM-1 is present in atherosclerotic plaques, and may be released into the bloodstream upon plaque rupture. Additional ICAM molecules are well known in the art, including ICAM-2 (also called CD102) and ICAM-3 (also called CD50), which may also be present in the blood.

TREM-1 is an activating receptor expressed at high levels on neutrophils and monocytes that infiltrate human tissues infected with bacteria. It is upregulated on peritoneal neutrophils of patients with microbial sepsis and mice with experimental lipopolysaccaride (LPS)-induced shock. It has been suggested to be a diagnostic marker of sepsis. See, e.g., Gibot et al., Ann. Int. Med. 141: 9-15, 2004.

Vascular cell adhesion molecule (VCAM), also called CD106, is a 100-110 kDa cell surface-bound immunoglobulin-like integrin ligand that facilitates binding of B lymphocytes and developing T lymphocytes to antigen-presenting cells during lymphocyte recruitment. VCAM is normally produced by endothelial cells, which line blood and lymph vessels, the heart, and other body cavities. VCAM-1 can be released from the cell surface during cell death or as a result of proteolytic activity. The normal serum concentration of sVCAM is approximately 650 ng/ml (6.5 nM). The plasma concentration of sVCAM-1 is marginally elevated in patients with acute myocardial infarction, unstable angina, and stable angina (Mulvihill, N. et al., Am. J. Cardiol. 83:1265-7, A9, 1999; Ghaisas, N. K. et al., Am. J. Cardiol. 80:617-619, 1997). However, sVCAM-1 is expressed in atherosclerotic lesions and its plasma concentration may correlate with the extent of atherosclerosis (Iiyama, K. et al., Circ. Res. 85:199-207, 1999; Peter, K. et al., Arterioscler. Thromb. Vasc. Biol. 17:505-512, 1997). Elevations in the plasma concentration of sVCAM-1 are associated with ischemic stroke, cancer, diabetes, preeclampsia, vascular injury, and other nonspecific inflammatory states (Bitsch, A. et al., Stroke 29:2129-2135, 1998; Otsuki, M. et al., Diabetes 46:2096-2101, 1997; Banks, R. E. et al., Br. J. Cancer 68:122-124, 1993; Steiner, M. et al., Thromb. Haemost. 72:979-984, 1994; Austgulen, R. et al., Eur. J. Obstet. Gynecol. Reprod. Biol. 71:53-58, 1997).

“Prolyl-specific dipeptidyl peptidase” or “prolyl-specific DPP” refer to serine proteases that cleave dipeptides from the N-terminal of substrate polypeptides, and that exhibit a preference for proline in the second position (i.e., NH2-X-pro-peptide-COOH, where X is an amino acid, and the bond between pro and the remaining peptide is cleaved). Such proteases are generally classified under E.C.3.4.14.X, including E.C.3.4.14.5 and 3.4.14.11. DPPs are often classified into types such as DPP-II and DPP-IV. DPP-IV, also called CD26, is a Type II membrane protein, and also exists in a soluble form that differs substantially from the membrane bound equivalent. See, e.g., U.S. Pat. No. 6,265,551.

Monocyte chemotactic protein-I (MCP-1) is a 10 kDa chemotactic factor that attracts monocytes and basophils, but not neutrophils or eosiniphils. MCP-1 is normally found in equilibrium between a monomeric and homodimeric form, and it is normally produced in and secreted by monocytes and vascular endothelial cells (Yoshimura, T. et al., FEBS Lett. 244:487-493, 1989; Li, Y. S. et al., Mol. Cell. Biochem. 126:61-68, 1993). MCP-1 has been implicated in the pathogenesis of a variety of diseases that involve monocyte infiltration, including psoriasis, rheumatoid arthritis, and atherosclerosis. The normal concentration of MCP-1 in plasma is <0.1 ng/ml. The plasma concentration of MCP-1 is elevated in patients with acute myocardial infarction, and may be elevated in the plasma of patients with unstable angina, but no elevations are associated with stable angina (Soejima, H. et al., J. Am. Coll. Cardiol. 34:983-988, 1999; Nishiyama, K. et al., Jpn. Circ. J. 62:710-712, 1998; Matsumori, A. et al., J. Mol. Cell. Cardiol. 29:419-423, 1997). Interestingly, MCP-1 also may be involved in the recruitment of monocytes into the arterial wall during atherosclerosis. Elevations of the serum concentration of MCP-1 are associated with various conditions associated with inflammation, including alcoholic liver disease, interstitial lung disease, sepsis, and systemic lupus erythematosus (Fisher, N. C. et al., Gut 45:416-420, 1999; Suga, M. et al., Eur. Respir. J. 14:376-382, 1999; Bossink, A. W. et al., Blood 86:3841-3847, 1995; Kaneko, H. et al. J. Rheumatol. 26:568-573, 1999). MCP-1 is released into the bloodstream upon activation of monocytes and endothelial cells. The concentration of MCP-1 in plasma form patients with acute myocardial infarction has been reported to approach 1 ng/ml (100 pM), and can remain elevated for one month (Soejima, H. et al., J. Am. Coll. Cardiol. 34:983-988, 1999). MCP-1 is a specific marker of the presence of a pro-inflammatory condition that involves monocyte migration.

Macrophage migration inhibitory factor (MIF) is a lymphokine involved in cell-mediated immunity, immunoregulation, and inflammation. It plays a role in the regulation of macrophage function in host defense through the suppression of anti-inflammatory effects of glucocorticoids. Monocytes and macrophages are reported to be a significant source of MIF after stimulation with endotoxin (lipopolysaccharide, or LPS) or with the cytokines tumor necrosis factor α (TNFα) and interferon-γ (IFNγ). MIF also was described to mediate certain pro-inflammatory effects, stimulating macrophages to produce TNFα and nitric oxide when given in combination with IFNγ (8, 9). Like TNFα and IL-1β, MIF plays a central role in the host response to endotoxemia. Coinjection of recombinant MIF and LPS exacerbates LPS lethality, whereas neutralizing anti-MIF antibodies fully protect mice from endotoxic shock.

Hemoglobin (Hb) is an oxygen-carrying iron-containing globular protein found in erythrocytes. It is a heterodimer of two globin subunits. α₂γ₂ is referred to as fetal Hb, α₂β₂ is called adult HbA, and α₂δ₂ is called adult HbA₂. 90-95% of hemoglobin is HbA, and the α₂ globin chain is found in all Hb types, even sickle cell hemoglobin. Hb is responsible for carrying oxygen to cells throughout the body. Hbα₂ is not normally detected in serum.

Human lipocalin-type prostaglandin D synthase (hPDGS), also called β-trace, is a 30 kDa glycoprotein that catalyzes the formation of prostaglandin D2 from prostaglandin H. The upper limit of hPDGS concentrations in apparently healthy individuals is reported to be approximately 420 ng/ml (Patent No. EP0999447A1). Elevations of hPDGS have been identified in blood from patients with unstable angina and cerebral infarction (Patent No. EP0999447A1). Furthermore, hPDGS appears to be a useful marker of ischemic episodes, and concentrations of hPDGS were found to decrease over time in a patient with angina pectoris following percutaneous transluminal coronary angioplasty (PTCA), suggesting that the hPGDS concentration decreases as ischemia is resolved (Patent No. EP0999447A1).

Mast cell tryptase, also known as alpha tryptase, is a 275 amino acid (30.7 kDa) protein that is the major neutral protease present in mast cells. Mast cell tryptase is a specific marker for mast cell activation, and is a marker of allergic airway inflammation in asthma and in allergic reactions to a diverse set of allergens. See, e.g., Taira et al., J. Asthma 39: 315-22 (2002); Schwartz et al., N. Engl. J. Med. 316: 1622-26 (1987). Elevated serum tryptase levels (>1 ng/mL) between 1 and 6 hours after an event provides a specific indication of mast cell degranulation.

Eosinophil cationic protein (ECP) is a heterogeneous protein with molecular weight variants from 16-24 kDa and a pI of pH 10.8. ECP is highly cytotoxic and is released by activated eosinophils. Venge, Clinical and experimental allergy, 23 (suppl. 2): 3-7 (1993). Concentrations of ECP in the bronchoalveolar lavage fluid (BALF) of asthma patients vary with the severity of their disease, and ECP concentrations in sputum have also been shown to reflect the pathophysiology of the disease. Bousquet et al., New Engl. J Med. 323: 1033-9 (1990). Virchow et al., Am. Rev. Respir. Dis. 146: 604-6 (1992). Assessment of serum ECP may be assumed to reflect pulmonary inflammation in bronchial asthma. Koller et al., Arch. Dis. Childhood 73: 413-7 (1995); see also, Sorkness et al., Clin. Exp. Allergy 32: 1355-59 (2002); Badr-elDin et al., East Mediterr. Health J. 5: 664-75 (1999).

KL-6 (also referred to as MUC1) is a high molecular weight (>300 kDa) mucinous glycoprotein expressed on pneumonocytes. Serum levels of KL-6 are reportedly elevated in interstitial lung diseases, which are characterized by exertional dyspnea. KL-6 has been shown to be a marker of various interstitial lung diseases, including pulmonary fibrosis, interstitial pneumonia, sarcoidosis, and interstitial pneumonitis. See, e.g., Kobayashi and Kitamura, Chest 108: 311-15 (1995); Kohno, J. Med. Invest. 46: 151-58 (1999); Bandoh et al., Ann. Rheum. Dis. 59: 257-62 (2000); and Yamane et al., J. Rheumatol. 27: 930-4 (2000).

(v) Exemplary Specific Markers for Neural Tissue Injury

Tissue injury markers, including markers of myocardial injury, vascular tissue injury, collagen synthesis and degradation, pulmonary injury, and neural tissue injury, may be particularly relevant to SIRS-related diseases, as organ dysfunction may be a hallmark of worsening or end-stage disease. The following provides an exemplary list of markers of neural tissue injury. Other exemplary tissue injury markers are described herein.

Adenylate kinase (AK) is a ubiquitous 22 kDa cytosolic enzyme that catalyzes the interconversion of ATP and AMP to ADP. Four isoforms of adenylate kinase have been identified in mammalian tissues (Yoneda, T. et al., Brain Res Mol Brain Res 62:187-195, 1998). The AK1 isoform is found in brain, skeletal muscle, heart, and aorta. The normal serum mass concentration of AKI is currently unknown, because a functional assay is typically used to measure total AK concentration. The normal serum AK concentration is <5 units/liter and AK elevations have been performed using CSF (Bollensen, E. et al., Acta Neurol Scand 79:53-582, 1989). Serum AK1 appears to have the greatest specificity of the AK isoforms as a marker of cerebral injury. AK may be best suited as a cerebrospinal fluid marker of cerebral ischemia, where its dominant source would be neural tissue.

Neurotrophins are a family of growth factors expressed in the mammalian nervous system. Some examples include nerve growth factor (NGF), brain-derived neurotrophic factor (BDNF), neurotrophin-3 (NT-3) and neurotrophin-4/5 (NT-4/5). Neurotrophins exert their effects primarily as target-derived paracrine or autocrine neurotrophic factors. The role of the neurotrophins in survival, differentiation and maintenance of neurons is well known. They exhibit partially overlapping but distinct patterns of expression and cellular targets. In addition to the effects in the central nervous system, neurotrophins also affect peripheral afferent and efferent neurons.

BDNF is a potent neurotrophic factor which supports the growth and survivability of nerve and/or glial cells. BDNF is expressed as a 32 kDa precursor “pro-BDNF” molecule that is cleaved to a mature BDNF form. Mowla et al., J. Biol. Chem. 276: 12660-6 (2001). The most abundant active form of human BDNF is a 27 kDa homodimer, formed by two identical 119 amino acid subunits, which is held together by strong hydrophobic interactions; however, pro-BDNF is also released extracellularly and is biologically active. BDNF is widely distributed throughout the CNS and displays in vitro trophic effects on a wide range of neuronal cells, including hippocampal, cerebellar, and cortical neurons. In vivo, BDNF has been found to rescue neural cells from traumatic and toxic brain injury. For example, studies have shown that after transient middle cerebral artery occlusion, BDNF mRNA is upregulated in cortical neurons (Schabiltz et al., J. Cereb. Blood Flow Metab. 14:500-506, 1997). In experimentally induced focal, unilateral thrombotic stroke, BDNF mRNA was increased from 2 to 18 h following the stroke. Such results suggest that BDNF potentially plays a neuroprotective role in focal cerebral ischemia.

NT-3 is also a 27 kDa homodimer consisting of two 119-amino acid subunits. The addition of NT-3 to primary cortical cell cultures has been shown to exacerbate neuronal death caused by oxygen-glucose deprivation, possible via oxygen free radical mechanisms (Bates et al., Neurobiol. Dis. 9:24-37, 2002). NT-3 is expressed as an inactive pro-NT-3 molecule, which is cleaved to the mature biologically active form.

Calbindin-D is a 28 kDa cytosolic vitamin D-dependent Ca²⁺-binding protein that may serve a cellular protective function by stabilizing intracellular calcium levels. Calbindin-D is found in the central nervous system, mainly in glial cells, and in cells of the distal renal tubule (Hasegawa, S. et al., J. Urol. 149:1414-1418, 1993). The normal serum concentration of calbindin-D is <20 pg/ml (0.7 pM). Serum calbindin-D concentration is reportedly elevated following cardiac arrest, and this elevation is thought to be a result of CNS damage due to cerebral ischemia (Usui, A. et al., J. Neurol. Sci. 123:134-139, 1994). Elevations of serum calbindin-D are elevated and plateau soon after reperfusion following ischemia. Maximum serum calbindin-D concentrations can be as much as 700 pg/ml (25 pM).

Creatine kinase (CK) is a cytosolic enzyme that catalyzes the reversible formation of ADP and phosphocreatine from ATP and creatine. The brain-specific CK isoform (CK-BB) is an 85 kDa cytosolic protein that accounts for approximately 95% of the total brain CK activity. It is also present in significant quantities in cardiac tissue, intestine, prostate, rectum, stomach, smooth muscle, thyroid uterus, urinary bladder, and veins (Johnsson, P. J., Cardiothorac. Vasc. Anesth. 10:120-126, 1996). The normal serum concentration of CK-BB is <10 ng/ml (120 pM). Serum CK-BB is elevated after hypoxic and ischemic brain injury, but a further investigation is needed to identify serum elevations in specific stroke types (Laskowitz, D. T. et al., J. Stroke Cerebrovasc. Dis. 7:234-241, 1998). Elevations of CK-BB in serum can be attributed to cerebral injury due to ischemia, coupled with increased permeability of the blood brain barrier. No correlation of the serum concentration of CK-BB with the extent of damage (infarct volume) or neurological outcome has been established. CK-BB has a half-life of 1-5 hours in serum and is normally detected in serum at a concentration of <10 ng/ml (120 pM). In severe stroke, serum concentrations CK-BB are elevated and peak soon after the onset of stroke (within 24 hours), gradually returning to normal after 3-7 days (4). CK-BB concentrations in the serum of individuals with head injury peak soon after injury and return to normal between 3.5-12 hours after injury, depending on the injury severity (Skogseid, I. M. et al., Acta Neurochir. (Wien.) 115:106-111, 1992). Maximum serum CK-BB concentrations can exceed 250 ng/ml (3 nM). CK-BB may be best suited as a CSF marker of cerebral ischemia, where its dominant source would be neural tissue. CK-BB might be more suitable as a serum marker of CNS damage after head injury because it is elevated for a short time in these individuals, with its removal apparently dependent upon the severity of damage.

Glial fibrillary acidic protein (GFAP) is a 55 kDa cytosolic protein that is a major structural component of astroglial filaments and is the major intermediate filament protein in astrocytes. GFAP is specific to astrocytes, which are interstitial cells located in the CNS and can be found near the blood-brain barrier. GFAP is not normally detected in serum. Serum GFAP is elevated following ischemic stroke (Niebroj-Dobosz, I., et al., Folia Neuropathol. 32:129-137, 1994). Current reports investigating serum GFAP elevations associated with stroke are severely limited, and much further investigation is needed to establish GFAP as a serum marker for all stroke types. Most studies investigating GFAP as a stroke marker have been performed using cerebrospinal fluid. Elevations of GFAP in serum can be attributed to cerebral injury due to ischemia, coupled with increased permeability of the blood brain barrier. No correlation of the serum concentration of GFAP with the extent of damage (infarct volume) or neurological outcome has been established. GFAP is elevated in cerebrospinal fluid of individuals with various neuropathies affecting the CNS, but there are no reports currently available describing the release of GFAP into the serum of individuals with diseases other than stroke (Albrechtsen, M. and Bock, E. J., Neuroimmunol. 8:301-309, 1985). Serum concentrations GFAP appear to be elevated soon after the onset of stroke, continuously increase and persist for an amount of time (weeks) that may correlate with the severity of damage. GFAP appears to a very specific marker for severe CNS injury, specifically, injury to astrocytes due to cell death caused by ischemia or physical damage.

Lactate dehydrogenase (LDH) is a ubiquitous 135 kDa cytosolic enzyme. It is a tetramer of A and B chains that catalyzes the reduction of pyruvate by NADH to lactate. Five isoforms of LDH have been identified in mammalian tissues, and the tissue-specific isoforms are made of different combinations of A and B chains. The normal serum mass concentration of LDH is currently unknown, because a functional assay is typically used to measure total LDH concentration. The normal serum LDH concentration is <600 units/liter (Ray, P. et al., Cancer Detect. Prev. 22:293-304, 1998). A great majority of investigations into LDH elevations in the context of stroke have been performed using cerebrospinal fluid, and elevations correlate with the severity of injury. Elevations in serum LDH activity are reported following both ischemic and hemorrhagic stroke, but further studies are needed in serum to confirm this observation and to determine a correlation with the severity of injury and neurological outcome (Aggarwal, S. P. et al., J. Indian Med. Assoc. 93:331-332, 1995; Maiuri, F. et al., Neurol. Res. 11:6-8, 1989). LDH may be best suited as a cerebrospinal fluid marker of cerebral ischemia, where its dominant source would be neural tissue.

Myelin basic protein (MBP) is actually a 14-21 kDa family of cytosolic proteins generated by alternative splicing of a single MBP gene that is likely involved in myelin compaction around axons during the myelination process. MBP is specific to oligodendrocytes in the CNS and in Schwann cells of the peripheral nervous system (PNS). It accounts for approximately 30% of the total myelin protein in the CNS and approximately 10% of the total myelin protein in the PNS. The normal serum concentration of MBP is <7 ng/ml (400 pM). Serum MBP is elevated after all types of severe stroke, specifically thrombotic stroke, embolic stroke, intracerebral hemorrhage, and subarachnoid hemorrhage, while elevations in MBP concentration are not reported in the serum of individuals with strokes of minor to moderate severity, which would include lacunar infarcts or transient ischemic attacks (Palfreyman, J. W. et al., Clin. Chim. Acta 92:403-409, 1979). Elevations of MBP in serum can be attributed to cerebral injury due to physical damage or ischemia caused by infarction or cerebral hemorrhage, coupled with increased permeability of the blood brain barrier. The serum concentration of MBP has been reported to correlate with the extent of damage (infarct volume), and it may also correlate with neurological outcome. The amount of available information regarding serum MBP elevations associated with stroke is limited, because most investigations have been performed using cerebrospinal fluid. MBP is normally detected in serum at an upper limit of 7 ng/ml (400 pM), is elevated after severe stroke and cerebral injury. Serum MBP is thought to be elevated within hours after stroke onset, with concentrations increasing to a maximum level within 2-5 days after onset. After the serum concentration reaches its maximum, which can exceed 120 ng/ml (6.9 nM), it can take over one week to gradually decrease to normal concentrations. Because the severity of damage has a direct effect on the release of MBP, it will affect the release kinetics by influencing the length of time that MBP is elevated in the serum. MBP will be present in the serum for a longer period of time as the severity of injury increases. The release of MBP into the serum of patients with head injury is thought to follow similar kinetics as those described for stroke, except that serum MBP concentrations reportedly correlate with the neurological outcome of individuals with head injury (Thomas, D. G. et al., Acta Neurochir. Suppl. (Wien) 28:93-95, 1979). The release of MBP into the serum of patients with intracranial tumors is thought to be persistent, but still needs investigation. Finally, serum MBP concentrations can sometimes be elevated in individuals with demyelinating diseases, but no conclusive investigations have been reported. As reported in individuals with multiple sclerosis, MBP is frequently elevated in the cerebrospinal fluid, but matched elevations in serum are often not present (Jacque, C. et al., Arch. Neurol. 39:557-560, 1982). This could indicate that cerebral damage has to be accompanied by an increase in the permeability of the blood-brain barrier to result in elevation of serum MBP concentrations. However, MBP can also be elevated in the population of individuals having intracranial tumors. The presence of these individuals in the larger population of individuals that would be candidates for an assay using this marker for stroke is rare. These individuals, in combination with individuals undergoing neurosurgical procedures or with demyelinating diseases, would nonetheless have an impact on determining the specificity of MBP for cerebral injury. Additionally, serum MBP may be useful as a marker of severe stroke, potentially identifying individuals that would not benefit from stroke therapies and treatments, such as tPA administration.

Neural cell adhesion molecule (NCAM), also called CD56, is a 170 kDa cell surface-bound immunoglobulin-like integrin ligand that is involved in the maintenance of neuronal and glial cell interactions in the nervous system, where it is expressed on the surface of astrocytes, oligodendrocytes, Schwann cells, neurons, and axons. NCAM is also localized to developing skeletal muscle myotubes, and its expression is upregulated in skeletal muscle during development, denervation and renervation. The normal serum mass concentration of NCAM has not been reported. NCAM is commonly measured by a functional enzyme immunoassay and is reported to have a normal serum concentration of <20 units/ml. Changes in serum NCAM concentrations specifically related to stroke have not been reported. NCAM may be best suited as a CSF marker of cerebral ischemia, where its dominant source would be neural tissue.

Enolase is a 78 kDa homo- or heterodimeric cytosolic protein produced from α, β, and γ subunits. It catalyzes the interconversion of 2-phosphoglycerate and phosphoenolpyruvate in the glycolytic pathway. Enolase can be present as αα, ββ, αγ, and γγ isoforms. The α subunit is found in glial cells and most other tissues, the β subunit is found in muscle tissue, and the y subunit if found mainly in neuronal and neuroendocrine cells (Quinn, G. B. et al., Clin. Chem. 40:790-795, 1994). The γγ enolase isoform is most specific for neurons, and is referred to as neuron-specific enolase (NSE). NSE, found predominantly in neurons and neuroendocrine cells, is also present in platelets and erythrocytes. The normal serum concentration of NSE is <12.5 ng/ml (160 pM).

NSE is made up of two subunits; thus, the most feasible immunological assay used to detect NSE concentrations would be one that is directed against one of the subunits. In this case, the γ subunit would be the ideal choice. However, the γ subunit alone is not as specific for cerebral tissue as the γγ isoform, since a measurement of the γ subunit alone would detect both the αγ and γγ isoforms. In this regard, the best immunoassay for NSE would be a two-site assay that could specifically detect the γγ isoform. Serum NSE is reportedly elevated after all stroke types, including TIAs, which are cerebral in origin and are thought to predispose an individual to having a more severe stroke at a later date (Isgro, F. et al., Eur. J. Cardiothorac. Surg. 11:640-644, 1997). Elevations of NSE in serum can be attributed to cerebral injury due to physical damage or ischemia caused by infarction or cerebral hemorrhage, coupled with increased permeability of the blood brain barrier, and the serum concentration of NSE has been reported to correlate with the extent of damage (infarct volume) and neurological outcome (Martens, P. et al., Stroke 29:2363-2366, 1998). Additionally, a secondary elevation of serum NSE concentration may be an indicator of delayed neuronal injury resulting from cerebral vasospasm (Laskowitz, D. T. et al., J. Stroke Cerebrovasc. Dis. 7, 234-241, 1998). NSE, which has a biological half-life of 48 hours and is normally detected in serum at an upper limit of 12.5 ng/ml (160 pM), is elevated after stroke and cerebral injury. Serum NSE is elevated after 4 hours from stroke onset, with concentrations reaching a maximum 1-3 days after onset (Missler, U. et al., Stroke 28:1956-1960, 1997). After the serum concentration reaches its maximum, which can exceed 300 ng/ml (3.9 nM), it gradually decreases to normal concentrations over approximately one week. Because the severity of damage has a direct effect on the release of NSE, it will affect the release kinetics by influencing the length of time that NSE is elevated in the serum. NSE will be present in the serum for a longer period of time as the severity of injury increases.

The release of NSE into the serum of patients with head injury follows different kinetics as seen with stroke, with the maximum serum concentration being reached within 1-6 hours after injury, often returning to baseline within 24 hours (Skogseid, I. M. et al., Acta Neurochir. (Wien.) 115:106-111, 1992). NSE is a specific marker for cerebral injury, specifically, injury to neuronal cells due to cell death caused by ischemia or physical damage. Neurons are about 10-fold less abundant in the brain than glial cells, so any cerebral injury coupled with increased permeability of the blood-brain barrier will have to occur in a region that has a significant regional population of neurons to significantly increase the serum NSE concentration. In addition, elevated serum concentrations of NSE can also indicate complications related to cerebral injury after AMI and cardiac surgery. Elevations in the serum concentration of NSE correlate with the severity of damage and the neurological outcome of the individual. NSE can be used as a marker of all stroke types, including TIAs.

Proteolipid protein (PLP) is a 30 kDa integral membrane protein that is a major structural component of CNS myelin. PLP is specific to oligodendrocytes in the CNS and accounts for approximately 50% of the total CNS myelin protein in the central sheath, although extremely low levels of PLP have been found (<1%) in peripheral nervous system (PNS) myelin. The normal serum concentration of PLP is <9 ng/ml (300 pM). Serum PLP is elevated after cerebral infarction, but not after transient ischemic attack (Trotter, J. L. et al., Ann. Neurol. 14:554-558, 1983). Current reports investigating serum PLP elevations associated with stroke are severely limited. Elevations of PLP in serum can be attributed to cerebral injury due to physical damage or ischemia caused by infarction or cerebral hemorrhage, coupled with increased permeability of the blood brain barrier. Correlation of the serum concentration of PLP with the extent of damage (infarct volume) or neurological outcome has not been established. No investigations examining the release kinetics of PLP into serum and its subsequent removal have been reported, but maximum concentrations approaching 60 ng/ml (2 nM) have been reported in encephalitis patients, which nearly doubles the concentrations found following stroke. PLP appears to a very specific marker for severe CNS injury, specifically, injury to oligodendrocytes. The available information relating PLP serum elevations and stroke is severely limited. PLP is also elevated in the serum of individuals with various neuropathies affecting the CNS. The undiagnosed presence of these individuals in the larger population of individuals that would be candidates for an assay using this marker for stroke is rare.

S-100 is a 21 kDa homo- or heterodimeric cytosolic Ca²⁺-binding protein produced from α and β subunits. It is thought to participate in the activation of cellular processes along the Ca2+-dependent signal transduction pathway (Bonfrer, J. M. et al., Br. J. Cancer 77:2210-2214, 1998). S-100ao (αα isoform) is found in striated muscles, heart and kidney, S-100a (αβ isoform) is found in glial cells, but not in Schwann cells, and S-100(ββ isoform) is found in high concentrations in glial cells and Schwann cells, where it is a major cytosolic component. The β subunit is specific to the nervous system, predominantly the CNS, under normal physiological conditions and, in fact, accounts for approximately 96% of the total S-100 protein found in the brain (Jensen, R. et al., J. Neurochem. 45:700-705, 1985). In addition, S-100β can be found in tumors of neuroendocrine origin, such as gliomas, melanomas, Schwannomas, neurofibromas, and highly differentiated neuroblastomas, like ganglioneuroblastoma and ganglioneuroma (Persson, L. et al., Stroke 18:911-918, 1987). The normal serum concentration of S-100β is <0.2 ng/ml (19 pM), which is the detection limit of the immunological detection assays used. Serum S-100β is elevated after all stroke types, including TIAs. Elevations of S-100β in serum can be attributed to cerebral injury due to physical damage or ischemia caused by infarction or cerebral hemorrhage, coupled with increased permeability of the blood-brain barrier, and the serum concentration of S-100b has been shown to correlate with the extent of damage (infarct volume) and neurological outcome (Martens, P. et al., Stroke 29:2363-2366, 1998; Missler, U. et al., Stroke 28:1956-1960, 1997).

S-100b has a biological half-life of 2 hours and is not normally detected in serum, but is elevated after stroke and cerebral injury. Serum S-100β is elevated after 4 hours from stroke onset, with concentrations reaching a maximum 2-3 days after onset. After the serum concentration reaches its maximum, which can approach 20 ng/ml (1.9 mM), it gradually decreases to normal over approximately one week. Because the severity of damage has a direct effect on the release of S-100b, it will affect the release kinetics by influencing the length of time that S-100b is elevated in the serum. S-100b will be present in the serum for a longer period of time as the seventy of injury increases. The release of S-100b into the serum of patients with head injury seems to follow somewhat similar kinetics as reported with stroke, with the only exception being that serum S-100β can be detected within 2.5 hours of onset and the maximum serum concentration is reached approximately 1 day after onset (Woertgen, C. et al., Acta Neurochir. (Wien) 139:1161-1164, 1997). S-100β is a specific marker for cerebral injury, specifically, injury to glial cells due to cell death caused by ischemia or physical damage. Glial cells are about 10 times more abundant in the brain than neurons, so any cerebral injury coupled with increased permeability of the blood-brain barrier will likely produce elevations of serum S-100β. Furthermore, elevated serum concentrations of S-100b can indicate complications related to cerebral injury after AMI and cardiac surgery. S-100b has been virtually undetectable in normal individuals, and elevations in its serum concentration correlate with the seventy of damage and the neurological outcome of the individual. S-100b can be used as a marker of all stroke types, including TIAs.

Thrombomodulin (TM) is a 70 kDa single chain integral membrane glycoprotein found on the surface of vascular endothelial cells. TM demonstrates anticoagulant activity by changing the substrate specificity of thrombin. The formation of a 1:1 stoichiometric complex between thrombin and TM changes thrombin function from procoagulant to anticoagulant. This change is facilitated by a change in thrombin substrate specificity that causes thrombin to activate protein C (an inactivator of factor Va and factor VIIIa), but not cleave fibrinogen or activate other coagulation factors (Davie, E. W. et al., Biochem. 30:10363-10370, 1991). The normal serum concentration of TM is 25-60 ng/ml (350-850 pM). Current reports describing serum TM concentration alterations following ischemic stroke are mixed, reporting no changes or significant increases (Seki, Y. et al., Blood Coagul. Fibrinolysis 8:391-396, 1997). Serum elevations of TM concentration reflect endothelial cell injury and would not indicate coagulation or fibrinolysis activation.

The gamma isoform of protein kinase C (PKCg) is specific for CNS tissue and is not normally found in the circulation. PKCg is activated during cerebral ischemia and is present in the ischemic penumbra at levels 2-24-fold higher than in contralateral tissue, but is not elevated in infarcted tissue (Krupinski, J. et al., Acta Neurobiol. Exp. (Warz) 58:13-21, 1998). In addition, animal models have identified increased levels of PKCg in the peripheral circulation of rats following middle cerebral artery occlusion (Cornell-Bell, A. et al., Patent No. WO 01/16599 A1). Additional isoforms of PKC, beta I and beta II were found in increased levels in the infarcted core of brain tissue from patients with cerebral ischemia (Krupinski, J. et al., Acta Neurobiol. Exp. (Warz). 58:13-21, 1998). Furthermore, the alpha and delta isoforms of PKC (PKCa and PKCd, respectively) have been implicated in the development of vasospasm following subarachnoid hemorrhage using a canine model of hemorrhage. PKCd expression was significantly elevated in the basilar artery during the early stages of vasospasm, and PKCa was significantly elevated as vasospasm progressed (Nishizawa, S. et al., Eur. J. Pharmacol. 398:113-119, 2000). Therefore, it may be of benefit to measure various isoforms of PKC, either individually or in various combinations thereof, for the identification of cerebral damage, the presence of the ischemic penumbra, as well as the development and progression of cerebral vasospasm following subarachnoid hemorrhage. Ratios of PKC isoforms such as PKCg and either PKCbI, PKCbII, or both also may be of benefit in identifying a progressing stroke, where the ischemic penumbra is converted to irreversibly damaged infarcted tissue. In this regard, PKCg may be used to identify the presence and volume of the ischemic penumbra, and either PKCbI, PKCbII, or both may be used to identify the presence and volume of the infarcted core of irreversibly damaged tissue during stroke. PKCd, PKCa, and ratios of PKCd and PKCa may be useful in identifying the presence and progression of cerebral vasospasm following subarachnoid hemorrhage.

(vi) Other Non-Specific Markers for Tissue Injury

Human vascular endothelial growth factor (VEGF) is a dimeric protein, the reported activities of which include stimulation of endothelial cell growth, angiogenesis, and capillary permeability. VEGF is secreted by a variety of vascularized tissues. In an oxygen-deficient environment, vascular endothelial cells may be damaged and may not ultimately survive. However, such endothelial damage stimulates VEGF production by vascular smooth muscle cells. Vascular endothelial cells may exhibit increased survival in the presence of VEGF, an effect that is believed to be mediated by expression of Bc1-2. VEGF can exist as a variety of splice variants known as VEGF(189), VEGF(165), VEGF(164), VEGFB(155), VEGF(148), VEGF(145), and VEGF(121). Related molecules include prokineticin (EG-VEGF).

Insulin-like growth factor-1 (IGF-1) is a ubiquitous 7.5 kDa secreted protein that mediates the anabolic and somatogenic effects of growth hormone during development (1, 2). In the circulation, IGF-1 is normally bound to an IGF-binding protein that regulates IGF activity. The normal serum concentration of IGF-1 is approximately 160 ng/ml (21.3 nM). Serum IGF-1 concentrations are reported to be significantly decreased in individuals with ischemic stroke, and the magnitude of reduction appears to correlate with the severity of injury (Schwab, S. et al., Stroke 28:1744-1748, 1997). Decreased IGF-1 serum concentrations have been reported in individuals with trauma and massive activation of the immune system. Due to its ubiquitous expression, serum IGF-1 concentrations could also be decreased in cases of non-cerebral ischemia. Interestingly, IGF-1 serum concentrations are decreased following ischemic stroke, even though its cellular expression is upregulated in the infarct zone (Lee, W. H. and Bondy, C., Ann. N. Y. Acad. Sci. 679:418-422, 1993). The decrease in serum concentration could reflect an increased demand for growth factors or an increased metabolic clearance rate. Serum levels were significantly decreased 24 hours after stroke onset, and remained decreased for over 10 days (Schwab, S. et al., Stroke 28:1744-1748, 1997). Serum IGF-1 may be a sensitive indicator of cerebral injury. However, the ubiquitous expression pattern of IGF-1 indicates that all tissues can potentially affect serum concentrations of IGF-1, compromising the specificity of any assay using IGF-1 as a marker for stroke. In this regard, IGF-1 may be best suited as a cerebrospinal fluid marker of cerebral ischemia, where its dominant source would be neural tissue.

Adhesion molecules are involved in the inflammatory response can also be considered as acute phase reactants, as their expression levels are altered as a result of insult. Examples of such adhesion molecules include E-selectin, intercellular adhesion molecule-1, vascular cell adhesion molecule, and the like.

E-selectin, also called ELAM-1 and CD62E, is a 140 kDa cell surface C-type lectin expressed on endothelial cells in response to IL-1 and TNFα that mediates the “rolling” interaction of neutrophils with endothelial cells during neutrophil recruitment. The normal serum concentration of E-selectin is approximately 50 ng/ml (2.9 nM). Investigations into the changes on serum E-selectin concentrations following stroke have reported mixed results. Some investigations report increases in serum E-selectin concentration following ischemic stroke, while others find it unchanged (Bitsch, A. et al., Stroke 29:2129-2135, 1998; Kim, J. S., J. Neurol. Sci. 137:69-78, 1996; Shyu, K. G. et al., J. Neurol. 244:90-93, 1997). E-selectin concentrations are elevated in the CSF of individuals with subarachnoid hemorrhage and may predict vasospasm (Polin, R. S. et al., J. Neurosurg. 89:559-567, 1998). Elevations in the serum concentration of E-selectin would indicate immune system activation. Serum E-selectin concentrations are elevated in individuals with, atherosclerosis, various forms of cancer, preeclampsia, diabetes, cystic fibrosis, AMI, and other nonspecific inflammatory states (Hwang, S. J. et al., Circulation 96:4219-4225, 1997; Banks, R. E. et al., Br. J. Cancer 68:122-124, 1993; Austgulen, R. et al., Eur. J. Obstet. Gynecol. Reprod Biol. 71:53-58, 1997; Steiner, M. et al., Thromb. Haemost. 72:979-984, 1994; De Rose, V. et al., Am. J. Respir. Crit. Care Med. 157:1234-1239, 1998). The serum concentration of E-selectin may be elevated following ischemic stroke, but it is not clear if these changes are transient or regulated by an as yet unidentified mechanism. Serum E-selectin may be a specific marker of endothelial cell injury. It is not, however, a specific marker for stroke or cerebral injury, since it is elevated in the serum of individuals with various conditions causing the generation of an inflammatory state. Furthermore, elevation of serum E-selectin concentration is associated with some of the risk factors associated with stroke.

Head activator (HA) is an 11 amino acid, 1.1 kDa neuropeptide that is found in the hypothalamus and intestine. It was originally found in the freshwater coelenterate hydra, where it acts as a head-specific growth and differentiation factor. In humans, it is thought to be a growth regulating agent during brain development. The normal serum HA concentration is <0.1 ng/ml (100 pM) Serum HA concentration is persistently elevated in individuals with tumors of neural or neuroendocrine origin (Schaller, H. C. et al., J Neurooncol. 6:251-258, 1988; Winnikes, M. et al., Eur. J. Cancer 28:421-424, 1992). No studies have been reported regarding HA serum elevations associated with stroke. HA is presumed to be continually secreted by tumors of neural or neuroendocrine origin, and serum concentration returns to normal following tumor removal. Serum HA concentration can exceed 6.8 ng/ml (6.8 nM) in individuals with neuroendocrine-derived tumors. The usefulness of HA as part of a stroke panel would be to identify individuals with tumors of neural or neuroendocrine origin. These individuals may have serum elevations of markers associated with cerebral injury as a result of cancer, not cerebral injury related to stroke. Although these individuals may be a small subset of the group of individuals that would benefit from a rapid diagnostic of cerebral injury, the use of HA as a marker would aid in their identification. Finally, angiotensin converting enzyme, a serum enzyme, has the ability to degrade HA, and blood samples would have to be drawn using EDTA as an anticoagulant to inhibit this activity.

Glycated hemoglobin HbA1c measurement provides an assessment of the degree to which blood glucose has been elevated over an extended time period, and so has been related to the extent diabetes is controlled in a patient. Glucose binds slowly to hemoglobin A, forming the A1c subtype. The reverse reaction, or decomposition, proceeds relatively slowly, so any buildup persists for roughly 4 weeks. With normal blood glucose levels, glycated hemoglobin is expected to be 4.5% to 6.7%. As blood glucose concentration rise, however, more binding occurs. Poor blood sugar control over time is suggested when the glycated hemoglobin measure exceeds 8.0%.

(vii) Markers Related to Apoptosis

Caspase-3, also called CPP-32, YAMA, and apopain, is an interleukin-1β converting enzyme (ICE)-like intracellular cysteine proteinase that is activated during cellular apoptosis. Caspase-3 is present as an inactive 32 kda precursor that is proteolytically activated during apoptosis induction into a heterodimer of 20 kDa and 11 kDa subunits (Fernandes-Alnemri, T. et al., J. Biol. Chem. 269:30761-30764, 1994). Its cellular substrates include poly(ADP-ribose) polymerase (PARP) and sterol regulatory element binding proteins (SREBPs) (Liu, X. et al., J. Biol. Chem. 271:13371-13376, 1996). The normal plasma concentration of caspase-3 is unknown. There are no published investigations into changes in the plasma concentration of caspase-3 associated with ACS. There are increasing amounts of evidence supporting the hypothesis of apoptosis induction in cardiac myocytes associated with ischemia and hypoxia (Saraste, A., Herz 24:189-195, 1999; Ohtsuka, T. et al., Coron. Artery Dis. 10:221-225, 1999; James, T. N., Coron. Artery Dis. 9:291-307, 1998; Bialik, S. et al., J. Clin. Invest. 100:1363-1372, 1997; Long, X. et al., J. Clin. Invest. 99:2635-2643, 1997). Elevations in the plasma caspase-3 concentration may be associated with any physiological event that involves apoptosis. There is evidence that suggests apoptosis is induced in skeletal muscle during and following exercise and in cerebral ischemia (Carraro, U. and Franceschi, C., Aging (Milano) 9:19-34, 1997; MacManus, J. P. et al., J. Cereb. Blood Flow Metab. 19:502-510, 1999).

Cathepsin D (E.C.3.4.23.5.) is a soluble lysosomal aspartic proteinase. It is synthesized in the endoplasmic reticulum as a preprocathepsin D. Having a mannose-6-phosphate tag, procathepsin D is recognized by a mannose-6-phosphate receptor. Upon entering into an acidic lysosome, the single-chain procathepsin D (52 KDa) is activated to cathepsin D and subsequently to a mature two-chain cathepsin D (31 and 14 KDa, respectively). The two mannose-6-phosphate receptors involved in the lysosomal targeting of procathepsin D are expressed both intracellularly and on the outer cell membrane. The glycosylation is believed to be crucial for normal intracellular trafficking. The fundamental role of cathepsin D is to degrade intracellular and internalized proteins. Cathepsin D has been suggested to take part in antigen processing and in enzymatic generation of peptide hormones. The tissue-specific function of cathepsin D seems to be connected to the processing of prolactin. Rat mammary glands use this enzyme for the formation of biologically active fragments of prolactin. Cathepsin D is functional in a wide variety of tissues during their remodeling or regression, and in apoptosis.

Brain α spectrin (also referred to as a fodrin) is a cytoskeletal protein of about 284 kDa that interacts with calmodulin in a calcium-dependent manner. Like erythroid spectrin, brain a spectrin forms oligomers (in particular dimers and tetramers). Brain α spectrin contains two EF-hand domains and 23 spectrin repeats. The caspase 3-mediated cleavage of α spectrin during apoptotic cell death may play an important role in altering membrane stability and the formation of apoptotic bodies.

Other Preferred Markers

The following table provides a list of additional preferred markers, associated with a disease or condition for which each marker can provide useful information for differential diagnosis. As understood by the skilled artisan and described herein, markers may indicate different conditions when considered with additional markers in a panel; alternatively, markers may indicate different conditions when considered in the entire clinical context of the patient.

| Marker | Classification |
|---|---|
| Myoglobin | Tissue injury |
| E-selectin | Tissue injury |
| VEGF | Tissue injury |
| EG-VEGF | Tissue injury |
| Troponin I and complexes | Myocardial injury |
| Troponin T and complexes | Myocardial injury |
| Annexin V | Myocardial injury |
| B-enolase | Myocardial injury |
| CK-MB | Myocardial injury |
| Glycogen phosphorylase-BB | Myocardial injury |
| Heart type fatty acid binding protein | Myocardial injury |
| Phosphoglyceric acid mutase | Myocardial injury |
| S-100ao | Myocardial injury |
| ANP | Blood pressure regulation |
| CNP | Blood pressure regulation |
| Kininogen | Blood pressure regulation |
| CGRP II | Blood pressure regulation |
| urotensin II | Blood pressure regulation |
| BNP | Blood pressure regulation |
| calcitonin gene related peptide | Blood pressure regulation |
| arg-Vasopressin | Blood pressure regulation |
| Endothelin-1 (and/or Big ET-1) | Blood pressure regulation |
| Endothelin-2 (and/or Big ET-2) | Blood pressure regulation |
| Endothelin-3 (and/or Big ET-3) | Blood pressure regulation |
| procalcitonin | Blood pressure regulation |
| calcyphosine | Blood pressure regulation |
| adrenomedullin | Blood pressure regulation |
| aldosterone | Blood pressure regulation |
| angiotensin 1 | Blood pressure regulation |
| angiotensin 2 | Blood pressure regulation |
| angiotensin 3 | Blood pressure regulation |
| Bradykinin | Blood pressure regulation |
| Tachykinin-3 | Blood pressure regulation |
| calcitonin | Blood pressure regulation |
| Endothelin-2 | Blood pressure regulation |
| Endothelin-3 | Blood pressure regulation |
| Renin | Blood pressure regulation |
| Urodilatin | Blood pressure regulation |
| Ghrelin | Blood pressure regulation |
| Plasmin | Coagulation and hemostasis |
| Thrombin | Coagulation and hemostasis |
| Antithrombin-III | Coagulation and hemostasis |
| Fibrinogen | Coagulation and hemostasis |
| von Willebrand factor | Coagulation and hemostasis |
| D-dimer | Coagulation and hemostasis |
| PAI-1 | Coagulation and hemostasis |
| Protein C | Coagulation and hemostasis |
| Soluble Endothelial Protein C Receptor | Coagulation and hemostasis |
| (EPCR) | |
| TAFI | Coagulation and hemostasis |
| Fibrinopeptide A | Coagulation and hemostasis |
| Plasmin alpha 2 antiplasmin complex | Coagulation and hemostasis |
| Platelet factor 4 | Coagulation and hemostasis |
| Platelet-derived growth factor | Coagulation and hemostasis |
| P-selectin | Coagulation and hemostasis |
| Prothrombin fragment 1 + 2 | Coagulation and hemostasis |
| B-thromboglobulin | Coagulation and hemostasis |
| Thrombin antithrombin III complex | Coagulation and hemostasis |
| Thrombomodulin | Coagulation and hemostasis |
| Thrombus Precursor Protein | Coagulation and hemostasis |
| Tissue factor | Coagulation and hemostasis |
| Tissue factor pathway inhibitor-α | Coagulation and hemostasis |
| Tissue factor pathway inhibitor-β | Coagulation and hemostasis |
| basic calponin 1 | Vascular tissue |
| beta like 1 integrin | Vascular tissue |
| Calponin | Vascular tissue |
| CSRP2 | Vascular tissue |
| elastin | Vascular tissue |
| Endothelial cell-selective adhesion | Vascular tissue |
| molecule (ESAM) | |
| Fibrillin 1 | Vascular tissue |
| Junction Adhesion Molecule-2 | Vascular tissue |
| LTBP4 | Vascular tissue |
| smooth muscle myosin | Vascular tissue |
| transgelin | Vascular tissue |
| Carboxyterminal propeptide of type I | Collagen synthesis |
| procollagen (PICP) | |
| Collagen carboxyterminal telopeptide (ICTP) | Collagen degradation |
| APRIL (TNF ligand superfamily member 13) | Inflammatory |
| Complement C3a | Inflammatory |
| CCL-5 (RANTES) | Inflammatory |
| CCL-8 (MCP-2) | Inflammatory |
| CCL-19 (macrophage inflammatory | Inflammatory |
| protein-3β) | |
| CCL-20 (MIP-3α) | Inflammatory |
| CCL-23 (MIP-3) | Inflammatory |
| CXCL-13 (small inducible cytokine B13) | Inflammatory |
| CXCL-16 (small inducible cytokine B16) | Inflammatory |
| Glutathione S Transferase | Inflammatory |
| HIF 1 ALPHA | Inflammatory |
| IL-25 | Inflammatory |
| IL-23 | Inflammatory |
| IL-22 | Inflammatory |
| IL-18 | Inflammatory |
| IL-13 | Inflammatory |
| IL-12 | Inflammatory |
| IL-10 | Inflammatory |
| IL-1-Beta | Inflammatory |
| IL-1ra | Inflammatory |
| IL-4 | Inflammatory |
| IL-6 | Inflammatory |
| IL-8 | Inflammatory |
| Lysophosphatidic acid | Inflammatory |
| MDA-modified LDL | Inflammatory |
| Human neutrophil elastase | Inflammatory |
| C-reactive protein | Inflammatory |
| Insulin-like growth factor | Inflammatory |
| Inducible nitric oxide synthase | Inflammatory |
| Intracellular adhesion molecule | Inflammatory |
| Lipocalin-2 | Inflammatory |
| Lactate dehydrogenase | Inflammatory |
| MCP-1 | Inflammatory |
| MDA-LDL | Inflammatory |
| MMP-1 | Inflammatory |
| MMP-2 | Inflammatory |
| MMP-3 | Inflammatory |
| MMP-9 | Inflammatory |
| TIMP-1 | Inflammatory |
| TIMP-2 | Inflammatory |
| TIMP-3 | Inflammatory |
| n-acetyl aspartate | Inflammatory |
| PTEN | Inflammatory |
| Phospholipase A2 | Inflammatory |
| TNF Receptor Superfamily Member 1A | Inflammatory |
| Transforming growth factor beta | Inflammatory |
| TREM-1 | Inflammatory |
| TL-1 (TNF ligand related molecule-1) | Inflammatory |
| TL-1a | Inflammatory |
| Tumor necrosis factor alpha | Inflammatory |
| Vascular cell adhesion molecule | Inflammatory |
| Vascular endothelial growth factor | Inflammatory |
| cystatin C | Inflammatory |
| substance P | Inflammatory |
| Myeloperoxidase (MPO) | Inflammatory |
| macrophage inhibitory factor | Inflammatory |
| Fibronectin | Inflammatory |
| cardiotrophin 1 | Inflammatory |
| Haptoglobin | Inflammatory |
| PAPPA | Inflammatory |
| s-CD40 ligand | Inflammatory |
| HMG-1 (or HMGB1) | Inflammatory |
| IL-2 | Inflammatory |
| IL-4 | Inflammatory |
| IL-11 | Inflammatory |
| IL-13 | Inflammatory |
| IL-18 | Inflammatory |
| Eosinophil cationic protein | Inflammatory |
| Mast cell tryptase | Inflammatory |
| VCAM | Inflammatory |
| sICAM-1 | Inflammatory |
| TNFα | Inflammatory |
| Osteoprotegerin | Inflammatory |
| Prostaglandin D-synthase | Inflammatory |
| Prostaglandin E2 | Inflammatory |
| RANK ligand | Inflammatory |
| HSP-60 | Inflammatory |
| Serum Amyloid A | Inflammatory |
| s-iL 18 receptor | Inflammatory |
| S-iL-1 receptor | Inflammatory |
| s-TNF P55 | Inflammatory |
| s-TNF P75 | Inflammatory |
| sTLR-1 (soluble toll-like receptor-1) | Inflammatory |
| sTLR-2 | Inflammatory |
| sTLR-4 | Inflammatory |
| TGF-beta | Inflammatory |
| MMP-11 | Inflammatory |
| Beta NGF | Inflammatory |
| CD44 | Inflammatory |
| EGF | Inflammatory |
| E-selectin | Inflammatory |
| Fibronectin | Inflammatory |
| RAGE | Inflammatory |
| Neutrophil elastase | Pulmonary injury |
| KL-6 | Pulmonary injury |
| LAMP 3 | Pulmonary injury |
| LAMP3 | Pulmonary injury |
| Lung Surfactant protein A | Pulmonary injury |
| Lung Surfactant protein B | Pulmonary injury |
| Lung Surfactant protein C | Pulmonary injury |
| Lung Surfactant protein D | Pulmonary injury |
| phospholipase D | Pulmonary injury |
| PLA2G5 | Pulmonary injury |
| SFTPC | Pulmonary injury |
| MAPK10 | Neural tissue injury |
| KCNK4 | Neural tissue injury |
| KCNK9 | Neural tissue injury |
| KCNQ5 | Neural tissue injury |
| 14-3-3 | Neural tissue injury |
| 4.1B | Neural tissue injury |
| APO E4-1 | Neural tissue injury |
| myelin basic protein | Neural tissue injury |
| Atrophin 1 | Neural tissue injury |
| brain Derived neurotrophic factor | Neural tissue injury |
| Brain Fatty acid binding protein | Neural tissue injury |
| brain tubulin | Neural tissue injury |
| CACNA1A | Neural tissue injury |
| Calbindin D | Neural tissue injury |
| Calbrain | Neural tissue injury |
| Carbonic anhydrase XI | Neural tissue injury |
| CBLN1 | Neural tissue injury |
| Cerebellin 1 | Neural tissue injury |
| Chimerin 1 | Neural tissue injury |
| Chimerin 2 | Neural tissue injury |
| CHN1 | Neural tissue injury |
| CHN2 | Neural tissue injury |
| Ciliary neurotrophic factor | Neural tissue injury |
| CK-BB | Neural tissue injury |
| CRHR1 | Neural tissue injury |
| C-tau | Neural tissue injury |
| DRPLA | Neural tissue injury |
| GFAP | Neural tissue injury |
| GPM6B | Neural tissue injury |
| GPR7 | Neural tissue injury |
| GPR8 | Neural tissue injury |
| GRIN2C | Neural tissue injury |
| GRM7 | Neural tissue injury |
| HAPIP | Neural tissue injury |
| HIP2 | Neural tissue injury |
| LDH | Neural tissue injury |
| Myelin basic protein | Neural tissue injury |
| NCAM | Neural tissue injury |
| NT-3 | Neural tissue injury |
| NDPKA | Neural tissue injury |
| Neural cell adhesion molecule | Neural tissue injury |
| NEUROD2 | Neural tissue injury |
| Neurofiliment L | Neural tissue injury |
| Neuroglobin | Neural tissue injury |
| neuromodulin | Neural tissue injury |
| Neuron specific enolase | Neural tissue injury |
| Neuropeptide Y | Neural tissue injury |
| Neurotensin | Neural tissue injury |
| Neurotrophin 1, 2, 3, 4 | Neural tissue injury |
| NRG2 | Neural tissue injury |
| PACE4 | Neural tissue injury |
| phosphoglycerate mutase | Neural tissue injury |
| PKC gamma | Neural tissue injury |
| proteolipid protein | Neural tissue injury |
| PTEN | Neural tissue injury |
| PTPRZ1 | Neural tissue injury |
| RGS9 | Neural tissue injury |
| RNA Binding protein Regulatory Subunit | Neural tissue injury |
| S-100β | Neural tissue injury |
| SCA7 | Neural tissue injury |
| secretagogin | Neural tissue injury |
| SLC1A3 | Neural tissue injury |
| SORL1 | Neural tissue injury |
| SREB3 | Neural tissue injury |
| STAC | Neural tissue injury |
| STX1A | Neural tissue injury |
| STXBP1 | Neural tissue injury |
| Syntaxin | Neural tissue injury |
| thrombomodulin | Neural tissue injury |
| transthyretin | Neural tissue injury |
| adenylate kinase-1 | Neural tissue injury |
| BDNF | Neural tissue injury |
| neurokinin A | Neural tissue injury |
| neurokinin B | Neural tissue injury |
| s-acetyl Glutathione | apoptosis |
| cytochrome C | apoptosis |
| Caspase 3 | apoptosis |
| Cathepsin D | apoptosis |
| α-spectrin | apoptosis |

Ubiquitination and Sepsis

Ubiquitin-mediated degradation of proteins plays an important role in the control of numerous processes, such as the way in which extracellular materials are incorporated into a cell, the movement of biochemical signals from the cell membrane, and the regulation of cellular functions such as transcriptional on-off switches. The ubiquitin system has been implicated in the immune response and development. Ubiquitin is a 76-amino acid polypeptide that is conjugated to proteins targeted for degradation. The ubiquitin-protein conjugate is recognized by a 26S proteolytic complex that splits ubiquitin from the protein, which is subsequently degraded.

It has been reported that sepsis stimulates protein breakdown in skeletal muscle by a nonlysosomal energy-dependent proteolytic pathway, and because muscle levels of ubiquitin mRNA were also increased, the results were interpreted as indicating that sepsis-induced muscle protein breakdown is caused by upregulated activity of the energy-ubiquitin-dependent proteolytic pathway. The same proteolytic pathway has been implicated in muscle breakdown caused by denervation, fasting, acidosis, cancer, and burn injury. Thus, levels of ubiquitinated proteins generally, or of specific ubiquitin-protein conjugates or fragments thereof, can be measured as additional markers of the invention. See, Tiao et al., J. Clin. Invest. 99: 163-168, 1997. Moreover, circulating levels of ubiquitin itself can be a useful marker in the methods described herein. See, e.g., Majetschak et al., Blood 101: 1882-90, 2003.

Interestingly, ubiquitination of a protein or protein fragment may convert a non-specific marker into a more specific marker of sepsis. For example, muscle damage can increase the concentration of muscle proteins in circulation. But sepsis, by specifically upregulating the ubiquitination pathway, may result in an increase of ubiquitinated muscle proteins, thus distinguishing non-specific muscle damage from sepsis-induced muscle damage.

The skilled artisan will recognize that an assay for ubiquitin may be designed that recognizes ubiquitin itself, ubiquitin-protein conjugates, or both ubiquitin and ubiquitin-protein conjugates. For example, antibodies used in a sandwich immunoassay may be selected so that both the solid phase antibody and the labeled antibody recognize a portion of ubiquitin that is available for binding in both unconjugated ubiquitin and ubiquitin conjugates. Alternatively, an assay specific for ubiquitin conjugates of the muscle protein troponin could use one antibody (on a solid phase or label) that recognizes ubiquitin, and a second antibody (the other of the solid phase or label) that recognizes troponin.

The present invention contemplates measuring ubiquitin conjugates of any marker described herein. Preferred ubiquitin-muscle protein conjugates for detection as markers include, but are not limited to, troponin I-ubiquitin, troponin T-ubiquitin, troponin C-ubiquitin, binary and ternary troponin complex-ubiquitin, actin-ubiquitin, myosin-ubiquitin, tropomyosin-ubiquitin, and α-actinin-ubiquitin.

Exemplary SIRS Markers and Marker Panels

Exemplary markers and marker panels are preferably designed to diagnose sepsis, to differentiate sepsis, severe sepsis, septic shock and/or MODS from other causes of SIRS, to assist in the stratification of risk in sepsis patients, and most preferably to direct treatment of subjects. Particularly preferred markers are matrix metalloproteinase 9 (MMP-9), interleukin-1β (IL-1β), interleukin-6 (IL-6), interleukin-8 (IL-8), interleukin-10 (IL-10), interleukin-22 (IL-22), IL-1 receptor agonist (IL-1ra), CXCL6, CXCL13, CXCL16, CCL8, CCL20, CCL23, CCL26, D-dimer, HMG-1, tumor necrosis factor-α (TNF-α), B-type natriuretic protein (BNP), A-type natriuretic protein (ANP), C-type natriuretic protein (BNP), C-reactive protein (CRP), caspase-3, calcitonin, procalcitonin₃₋₁₁₆, soluble DPP-IV, soluble FAS ligand (sFasL), creatine kinase-BB (CK-BB), vascular endothelial growth factor (VEGF), myeloperoxidase (MPO), and soluble intercellular adhesion molecule-1 (sICAM-1), or immunologically detectable fragments of these proteins or their biosynthetic precursors.

These individual markers may also be grouped into marker panels. Preferred panels include one or more markers related to inflammation and one or more markers related to blood pressure regulation; one or more markers related to inflammation and one or more markers related to coagulation and hemostasis; or one or more markers related to inflammation, one or-more markers related to coagulation and hemostasis, and one or more markers related to blood pressure regulation.

Particularly preferred marker panels comprise a plurality of markers, and most preferably 3, 4, 5, 6, 7, 8, 9, 10, or more markers, selected from the group consisting of matrix metalloproteinase 9 (MMP-9), interleukin-1β (IL-1β), interleukin-6 (IL-6), interleukin-8 (IL-8), interleukin-10 (IL-10), interleukin-22 (IL-22), IL-1 receptor agonist (IL-1ra), CXCL6, CXCL13, CXCL16, CCL8, CCL20, CCL23, CCL26, D-dimer, HMG-1, tumor necrosis factor-α (TNF-α), B-type natriuretic protein (BNP), A-type natriuretic protein (ANP), C-type natriuretic protein (BNP), C-reactive protein (CRP), caspase-3, calcitonin, procalcitonin₃₋₁₁₆, soluble DPP-IV, soluble FAS ligand (sFasL), creatine kinase-BB (CK-BB), vascular endothelial growth factor (VEGF), myeloperoxidase (MPO), and soluble intercellular adhesion molecule-1 (sICAM-1), and immunologically detectable fragments thereof.

Assay Measurement Strategies

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwitch, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of markers may be carried out separately or simultaneously with one test sample. For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses or protein chips perform simultaneous assays of a plurality of markers on a single surface. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease status that includes, but is not limited to identifying the approximate time from onset of the event, the presence and amount of salvagable tissue, the appropriateness of drug therapies, the effectiveness of various therapies as indicated by reperfusion or resolution of symptoms, differentiation of the various types of ACS, identification of the severity of the event, identification of the disease severity, and identification of the patient's outcome, including risk of future events.

A panel consisting of the markers referenced above may be constructed to provide relevant information related to differential diagnosis. Such a panel may be constucted using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W. B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses. Other measurement strategies applicable to the methods described herein include chromatography (e.g., HPLC), mass spectrometry, receptor-based assays, and combinations of the foregoing.

Selection of Antibodies

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

Selecting a Treatment Regimen

Just as the potential causes of any particular nonspecific symptom may be a large and diverse set of conditions, the appropriate treatments for these potential causes may be equally large and diverse. However, once a diagnosis is obtained, the clinician can readily select a treatment regimen that is compatible with the diagnosis. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999. With regard to SIRS, sepsis, severe sepsis, and septic shock, recent guidelines provide additional information for the clinician. See, e.g., Dellinger et al., Crit. Care Med. 32: 858-73, 2004, which is hereby incorporated by reference in its entirety.

While the present invention may be used to determine if any SIRS-related (that is, applicable to SIRS, sepsis, severe sepsis, septic shock, and MODS) treatment should be undertaken at all, the invention is preferably used to assign a particular treatment regimen from amongst two or more possible choices of SIRS-related treatment regimens. For example, in exemplary embodiments, the present invention is used to determine if subjects should receive standard therapy or early goal-directed therapy. Thus, the methods and compositions described herein may be used to select one or more of the following treatments for inclusion in a therapy regimen
Administration of intravenous antibiotic therapy;
maintenance of a central venous pressure of 8-12 mm Hg;
administration of crystalloids and/or colloids, preferably to maintain such a central venous pressure;
maintenance of a mean arterial pressure of ≧65 mm Hg;
administration of one or more vasopressors (e.g., norepinephrine, dopamine, and/or vasopressin) and/or vasodilators (e.g., prostacyclin, pentoxifylline, N-acetyl-cysteine);
administration of one or more corticosteroids (e.g., hydrocortisone);
administration of recombinant activated protein C;
maintenance of a central venous oxygen saturation of ≧70%;
administration of transfused red blood cells to a hematocrit of at least 30%;
administration of one or more inotropics (e.g., dobutamine); and
administration of mechanical ventilation.

This list is not meant to be limiting. In addition, since the methods and compositions described herein provide prognostic information, the panels and markers of the present invention may be used to monitor a course of treatment. For example, inproved or worsened prognostic state may indicate that a particular treatment is or is not efficacious.

### EXAMPLES

The following examples serve to illustrate the present invention. These examples are in no way intended to limit the scope of the invention.

### Example 1

### Subject Population

The subjects in the following examples are a subset of those reported in Rivers et al., N. Engl. J. Med. 345: 1368-77, 2001, which is hereby incorporated in its entirety. Samples were obtained at admission, and the subjects were then subdivided into two random groups, one of which received standard sepsis therapies, the other of which received an early goal-directed therapy (“EGDT”) regimen devised by the authors. In general, blood specimens are collected by trained study personnel using EDTA as the anticoagulant and centrifuged for greater than or equal to 10 minutes. The plasma component is transferred into a sterile cryovial and frozen at −20° C. or colder. Clinical histories are available for each of the patients to aid in the statistical analysis of the assay data.

### Example 2

### Biochemical Analyses

Markers are measured using standard immunoassay techniques. These techniques involved the use of antibodies to specifically bind the protein targets. A monoclonal antibody directed against a selected marker is biotinylated using N-hydroxysuccinimide biotin (NHS-biotin) at a ratio of about 5 NHS-biotin moieties per antibody. The antibody-biotin conjugate is then added to wells of a standard avidin 384 well microtiter plate, and antibody conjugate not bound to the plate is removed. This forms the “anti-marker” in the microtiter plate. Another monoclonal antibody directed against the same marker is conjugated to alkaline phosphatase using succinimidyl 4-[N-maleimidomethyl]-cyclohexane-1-carboxylate (SMCC) and N-succinimidyl 3-[2-pyridyldithio]propionate (SPDP) (Pierce, Rockford, Ill.).

Immunoassays are performed on a TECAN Genesis RSP 200/8 Workstation. Biotinylated antibodies are pipetted into microtiter plate wells previously coated with avidin and incubated for 60 min. The solution containing unbound antibody is removed, and the wells washed with a wash buffer, consisting of 20 mM borate (pH 7.42) containing 150 mM NaCl, 0.1% sodium azide, and 0.02% Tween-20. The plasma samples (10 μl ) are pipeted into the microtiter plate wells, and incubated for 60 min. The sample is then removed and the wells washed with a wash buffer. The antibody-alkaline phosphatase conjugate is then added to the wells and incubated for an additional 60 min, after which time, the antibody conjugate is removed and the wells washed with a wash buffer. A substrate, (AttoPhos®, Promega, Madison, Wis.) is added to the wells, and the rate of formation of the fluorescent product was related to the concentration of the marker in the patient samples.

The markers analyzed are reported in the following examples using the following units: BNP—pg/ml; BNP₃₋₁₀₈—pg/ml; BNP₇₉₋₁₀₈—pg/ml; calcitonin—pg/ml; caspase-3—ng/ml; CK-BB—ng/ml; CRP—μg/ml; D-dimer—μg/ml; sFasL—ng/ml; sICAM-1—ng/ml; HMG-1—ng/ml; IL-10—pg/ml; IL-1β—pg/ml; IL-1ra—pg/ml; IL-6—pg/ml; IL-8—pg/ml; MMP-9—ng/ml; MPO—ng/ml; TNF-α—pg/ml; VEGF—pg/ml.

### Example 3

### Marker Panels for Assignment of Therapy in SIRS

Using the methods described in PCT application no. US03/41426, filed Dec. 23, 2003, exemplary panels for risk stratification is SIRS were identified. Starting with a large number of potential markers, an iterative procedure was applied. In this procedure, individual threshold concentrations for the markers are not used as cutoffs per se, but are used as values to which the assay values for each patient are compared and normalized. Rather, a “window” of assay values between a minimum and maximum marker concentration (calculated as midpoint±midpoint×linear range in the tables below) is determined. Measured marker concentrations above the maximum are assigned a value of 1 and measured marker concentrations below the minimum are assigned a value of 0; measured marker concentrations within the window are linearly interpolated to a value of between 0 and 1. The value is then multiplied by a weighting factor (weight average in the tables below). The absolute values of the weights for all of the individual markers add up to 1. A negative weight for a marker implies that the assay values for the control group are higher than those for the diseased group. A “panel response” is calculated using the midpoint, linear range “window,” and weighting factors. The panel responses for the entire population of “disease group” and “controls” are subjected to ROC and/or correlation analysis, and a panel response cutoff is selected to yield the desired sensitivity and specificity for separating the “disease” and “non-disease” populations. After each set of iterations, the weakest contributors to the equation may be eliminated and the iterative process started again with the reduced number of markers. This process is continued until a minimum number of markers that will still result in acceptable sensitivity and specificity of the panel is obtained.

Using these methods, various panels may be defined, depending upon the identity of the markers selected, the number of markers for the final panel, and the selection of “disease” and “non-disease” populations for performing the optimization. The following exemplary panels provide panel response threholds used to analyse the indicated groups. These represent the panel response value used to best separate the two groups under study. Average ROC areas, sensitivities, and specificities calculated from 100 separate calculated “anneals” (together with the standard deviations of those averages), reported below, are used to determine the particular panel parameters.

1. Optimized to separate EGDT subjects surviving >28 days vs. normal therapy subjects surviving >28 days, 20 markers

| Marker ID | Midpoint | Linear Range | Weight |
|---|---|---|---|
| MMP-9 | 1184.28 | 0.48 | 0.09 |
| IL-6 | 3939.45 | 0.43 | 0.10 |
| IL-1ra | 3856.23 | 0.40 | 0.09 |
| D-Dimer | 11.75 | 0.38 | 0.06 |
| IL-10 | 182.47 | 0.35 | 0.07 |
| CK-BB | 1.98 | 0.59 | 0.06 |
| sFasL | 2.62 | 0.44 | 0.08 |
| MPO | 136.25 | 0.46 | 0.09 |
| CRP | 139.58 | 0.53 | 0.04 |
| VEGF | 2.03 | 0.42 | 0.07 |
| sICAM-1 | 493.67 | 0.34 | 0.06 |
| Calcitonin | 88.68 | 0.40 | 0.05 |
| Caspase-3 | 3.44 | 0.26 | 0.06 |
| BNP₃₋₁₀₈ | 2437.01 | 0.45 | 0.04 |
| BNP | 1324.72 | 0.49 | 0.05 |
| IL-8 | 312.54 | 0.42 | 0.06 |
| IL-1β | 84.52 | 0.76 | 0.05 |
| HMG-1 | 4.18 | 0.56 | 0.03 |
| TNF-α | 226.92 | 0.93 | 0.06 |
| BNP₇₉₋₁₀₈ | 96.53 | 0.72 | 0.06 |

Application to various subject groups:

| | | | | | Normal |
|---|---|---|---|---|---|
| | EGDT alive | EGDT alive | | EGDT alive | therapy alive |
| | v. normal | v. normal | Alive v. | v. EGDT | v. normal |
| | therapy, alive | therapy, dead | dead, entire | dead by | therapy dead |
| | >28 d. | by 28 d. | population | 28 d. | by 28 d. |
| N (disease)/N | 16/16 | 9/8 | 32/17 | 16/8 | 16/9 |
| (nondisease) | | | | | |
| Panel response | 0.205 | 0.314 | 0.259 | 0.237 | 0.294 |
| cutoff | | | | | |
| ROC area | 0.804 | 0.506 | 0.738 | 0.832 | 0.639 |
| Std dev | 0.034 | 0.090 | 0.043 | 0.043 | 0.076 |
| Sensitivity @ | 50.3% | 6.1% | 29.4% | 56.8% | 28.4% |
| 92.5% | | | | | |
| Specificity | | | | | |
| Std dev | 15.10% | 8.40% | 14.60% | 18.00% | 15.00% |
| Sensitivity @ | 58.6% | 8.3% | 40.4% | 54.0% | 19.1% |
| 92.5% | | | | | |
| Specificity | | | | | |
| Std dev | 10.5% | 10.2% | 11.5% | 16.8% | 14.3% |

2. Optimized to separate EGDT subjects surviving >28 days vs. normal therapy subjects surviving >28 days, 15 markers

| Marker ID | Midpoint | Linear Range | Weight |
|---|---|---|---|
| MMP-9 | 966.53 | 0.56 | 0.13 |
| IL-6 | 3920.09 | 0.56 | 0.12 |
| IL-1ra | 10681.38 | 0.45 | 0.08 |
| D-Dimer | 23.37 | 0.48 | 0.06 |
| IL-10 | 310.21 | 0.51 | 0.07 |
| CK-BB | 2.01 | 0.67 | 0.06 |
| sFasL | 3.56 | 0.40 | 0.10 |
| MPO | 85.21 | 0.53 | 0.11 |
| CRP | 148.45 | 0.46 | 0.07 |
| VEGF | 2.71 | 0.38 | 0.11 |
| sICAM-1 | 554.57 | 0.55 | 0.08 |
| Calcitonin | 102.73 | 0.47 | 0.04 |
| Caspase-3 | 3.63 | 0.35 | 0.06 |
| BNP₃₋₁₀₈ | 3895.88 | 0.74 | 0.05 |
| BNP | 1732.06 | 0.62 | 0.03 |

Application to various subject groups:

| | | | | | Normal |
|---|---|---|---|---|---|
| | EGDT alive | EGDT alive | | EGDT alive | therapy alive |
| | v. normal | v. normal | Alive v. | v. EGDT | v. normal |
| | therapy, alive | therapy, dead | dead, entire | dead by | therapy dead |
| | >28 d. | by 28 d. | population | 28 d. | by 28 d. |
| N (disease)/N | 29/27 | 20/14 | 56/34 | 29/14 | 27/20 |
| (nondisease) | | | | | |
| Panel response | 0.223 | 0.323 | 0.269 | 0.253 | 0.299 |
| cutoff | | | | | |
| ROC area | 0.796 | 0.590 | 0.652 | 0.818 | 0.489 |
| Std dev | 0.026 | 0.056 | 0.036 | 0.033 | 0.056 |
| Sensitivity @ | 46.3% | 12.9% | 12.6% | 48.6% | 9.0% |
| 92.5% | | | | | |
| Specificity | | | | | |
| Std dev | 8.50% | 9.90% | 7.30% | 12.90% | 7.20% |
| Sensitivity @ | 65.2% | 23.4% | 20.7% | 59.6% | 4.0% |
| 92.5% | | | | | |
| Specificity | | | | | |
| Std dev | 7.9% | 9.0% | 6.1% | 11.8% | 2.4% |

3. Optimized to separate EGDT subjects surviving >28 days vs. normal therapy subjects surviving >28 days, 11 markers

| Marker ID | Midpoint | Linear Range | Weight |
|---|---|---|---|
| MMP-9 | 1142.79 | 0.52 | 0.12 |
| IL-6 | 4296.18 | 0.47 | 0.18 |
| IL-1ra | 13801.80 | 0.40 | 0.12 |
| D-Dimer | 31.03 | 0.39 | 0.07 |
| IL-10 | 357.48 | 0.54 | 0.05 |
| CK-BB | 2.73 | 0.69 | 0.08 |
| sFasL | 3.96 | 0.51 | 0.10 |
| MPO | 125.75 | 0.54 | 0.13 |
| CRP | 118.28 | 0.51 | 0.07 |
| VEGF | 3.11 | 0.49 | 0.13 |
| sICAM-1 | 568.31 | 0.57 | 0.08 |

Application to various subject groups:

| | | | | | Normal |
|---|---|---|---|---|---|
| | EGDT alive | EGDT alive | | EGDT alive | therapy alive |
| | v. normal | v. normal | Alive v. | v. EGDT | v. normal |
| | therapy, alive | therapy, dead | dead, entire | dead by | therapy dead |
| | >28 d. | by 28 d. | population | 28 d. | by 28 d. |
| N (disease)/N | 30/31 | 21/14 | 61/35 | 30/14 | 31/21 |
| (nondisease) | | | | | |
| Panel response | 0.230 | 0.351 | 0.274 | 0.247 | 0.308 |
| cutoff | | | | | |
| ROC area | 0.813 | 0.578 | 0.620 | 0.813 | 0.449 |
| Std dev | 0.024 | 0.055 | 0.030 | 0.031 | 0.045 |
| Sensitivity @ | 47.7% | 15.2% | 4.9% | 43.1% | 2.2% |
| 92.5% | | | | | |
| Specificity | | | | | |
| Std dev | 9.60% | 9.00% | 4.60% | 12.00% | 3.30% |
| Sensitivity @ | 65.1% | 21.8% | 18.1% | 56.8% | 3.6% |
| 92.5% | | | | | |
| Specificity | | | | | |
| Std dev | 7.8% | 7.8% | 5.7% | 12.8% | 2.4% |

4. Optimized to separate EGDT subjects surviving >28 days vs. normal therapy subjects surviving >28 days, 8 markers

| Marker ID | Midpoint | Linear Range | Weight |
|---|---|---|---|
| MMP-9 | 1142.79 | 0.52 | 0.12 |
| IL-6 | 4296.18 | 0.47 | 0.18 |
| IL-1ra | 13801.80 | 0.40 | 0.12 |
| D-Dimer | 31.03 | 0.39 | 0.07 |
| CK-BB | 2.73 | 0.69 | 0.08 |
| sFasL | 3.96 | 0.51 | 0.10 |
| MPO | 125.75 | 0.54 | 0.13 |
| VEGF | 3.11 | 0.49 | 0.13 |

Application to various subject groups:

| | | | | | Normal |
|---|---|---|---|---|---|
| | EGDT alive | EGDT alive | | EGDT alive | therapy alive |
| | v. normal | v. normal | Alive v. | v. EGDT | v. normal |
| | therapy, alive | therapy, dead | dead, entire | dead by | therapy dead |
| | >28 d. | by 28 d. | population | 28 d. | by 28 d. |
| N (disease)/N | 42/38 | 27/22 | 80/49 | 42/22 | 38/27 |
| (nondisease) | | | | | |
| Panel response | 0.179 | 0.281 | 0.197 | 0.188 | 0.224 |
| cutoff | | | | | |
| ROC area | 0.719 | 0.598 | 0.629 | 0.771 | 0.478 |
| Std dev | 0.024 | 0.040 | 0.033 | 0.034 | 0.042 |
| Sensitivity @ | 32.9% | 19.5% | 8.8% | 34.0% | 2.3% |
| 92.5% | | | | | |
| Specificity | | | | | |
| Std dev | 5.10% | 8.30% | 4.40% | 9.60% | 2.80% |
| Sensitivity @ | 37.4% | 17.8% | 19.4% | 42.1% | 6.2% |
| 92.5% | | | | | |
| Specificity | | | | | |
| Std dev | 6.2% | 7.5% | 6.7% | 7.5% | 3.4% |

5. Optimized to separate EGDT alive >28 days vs. EGDT dead by 28 days, 20 markers

| Marker ID | Midpoint | Linear Range | Weight |
|---|---|---|---|
| MMP-9 | 923.92 | 0.43 | 0.07 |
| IL-6 | 2441.65 | 0.40 | 0.09 |
| IL-1ra | 3632.46 | 0.38 | 0.08 |
| D-Dimer | 8.75 | 0.44 | 0.10 |
| IL-10 | 270.88 | 0.38 | 0.07 |
| CK-BB | 1.49 | 0.52 | 0.07 |
| sFasL | 1.93 | 0.47 | 0.07 |
| MPO | 52.17 | 0.35 | 0.06 |
| CRP | 136.58 | 0.49 | 0.08 |
| VEGF | 0.98 | 0.52 | 0.07 |
| sICAM-1 | 717.17 | 0.32 | 0.08 |
| Calcitonin | 93.96 | 0.50 | 0.06 |
| Caspase-3 | 3.86 | 0.24 | 0.10 |
| BNP₃₋₁₀₈ | 5696.30 | 0.52 | 0.06 |
| BNP | 2557.61 | 0.55 | 0.06 |
| IL-8 | 279.36 | 0.41 | 0.12 |
| IL-1β | 74.48 | 0.39 | 0.06 |
| HMG-1 | 7.18 | 0.50 | 0.04 |
| TNF-α | 447.42 | 0.86 | 0.07 |
| BNP₇₉₋₁₀₈ | 111.26 | 0.53 | 0.05 |

Application to various subject groups:

| | | | | | Normal |
|---|---|---|---|---|---|
| | EGDT alive | EGDT alive | | EGDT alive | therapy alive |
| | v. normal | v. normal | Alive v. | v. EGDT | v. normal |
| | therapy, alive | therapy, dead | dead, entire | dead by | therapy dead |
| | >28 d. | by 28 d. | population | 28 d. | by 28 d. |
| N (disease)/N | 16/16 | 9/8 | 32/17 | 16/8 | 16/9 |
| (nondisease) | | | | | |
| Panel response | 0.257 | 0.399 | 0.363 | 0.377 | 0.329 |
| cutoff | | | | | |
| ROC area | 0.563 | 0.742 | 0.847 | 0.964 | 0.748 |
| Std dev | 0.058 | 0.129 | 0.034 | 0.022 | 0.073 |
| Sensitivity @ | 18.3% | 19.3% | 55.4% | 99.4% | 38.2% |
| 92.5% | | | | | |
| Specificity | | | | | |
| Std dev | 9.60% | 19.30% | 14.40% | 3.70% | 17.10% |
| Sensitivity @ | 27.1% | 55.6% | 51.2% | 93.8% | 32.7% |
| 92.5% | | | | | |
| Specificity | | | | | |
| Std dev | 8.8% | 17.1% | 13.6% | 3.1% | 16.1% |

6. Optimized to separate EGDT alive >28 days vs. EGDT dead by 28 days, 15 markers

| Marker ID | Midpoint | Linear Range | Weight |
|---|---|---|---|
| MMP-9 | 749.42 | 0.46 | 0.09 |
| IL-6 | 2316.43 | 0.51 | 0.10 |
| IL-1ra | 9607.05 | 0.45 | 0.07 |
| D-Dimer | 11.80 | 0.52 | 0.12 |
| IL-10 | 236.38 | 0.51 | 0.10 |
| CK-BB | 1.75 | 0.52 | 0.08 |
| sFasL | 1.48 | 0.35 | 0.08 |
| MPO | 175.24 | 0.41 | 0.07 |
| CRP | 145.78 | 0.35 | 0.07 |
| VEGF | 1.04 | 0.61 | 0.07 |
| sICAM-1 | 539.18 | 0.27 | 0.09 |
| IL-1β | 71.65 | 0.38 | 0.08 |
| IL-8 | 232.14 | 0.34 | 0.16 |
| BNP₃₋₁₀₈ | 7551.77 | 0.29 | 0.07 |
| BNP | 3436.47 | 0.48 | 0.06 |

Application to various subject groups:

| | | | | | Normal |
|---|---|---|---|---|---|
| | EGDT alive | EGDT alive | | EGDT alive | therapy alive |
| | v. normal | v. normal | Alive v. | v. EGDT | v. normal |
| | therapy, alive | therapy, dead | dead, entire | dead by | therapy dead |
| | >28 d. | by 28 d. | population | 28 d. | by 28 d. |
| N (disease)/N | 30/28 | 21/14 | 58/35 | 30/14 | 28/21 |
| (nondisease) | | | | | |
| Panel response | 0.292 | 0.389 | 0.350 | 0.357 | 0.336 |
| cutoff | | | | | |
| ROC area | 0.627 | 0.729 | 0.762 | 0.943 | 0.615 |
| Std dev | 0.044 | 0.054 | 0.026 | 0.012 | 0.046 |
| Sensitivity @ | 29.0% | 19.1% | 26.7% | 92.6% | 17.8% |
| 92.5% | | | | | |
| Specificity | | | | | |
| Std dev | 9.30% | 10.80% | 8.50% | 10.60% | 6.90% |
| Sensitivity @ | 17.9% | 50.8% | 25.4% | 92.3% | 10.8% |
| 92.5% | | | | | |
| Specificity | | | | | |
| Std dev | 10.1% | 9.9% | 10.0% | 2.1% | 5.9% |

7. Optimized to separate EGDT alive >28 days vs. EGDT dead by 28 days, 11 markers.

| Marker ID | Midpoint | Linear Range | Weight |
|---|---|---|---|
| MMP-9 | 817.00 | 0.38 | 0.09 |
| IL-6 | 2527.65 | 0.55 | 0.11 |
| IL-1ra | 11207.14 | 0.54 | 0.10 |
| D-Dimer | 12.22 | 0.47 | 0.14 |
| IL-10 | 247.84 | 0.50 | 0.12 |
| CK-BB | 1.97 | 0.47 | 0.10 |
| sFasL | 1.36 | 0.59 | 0.09 |
| IL-1β | 79.85 | 0.37 | 0.09 |
| CRP | 163.02 | 0.32 | 0.11 |
| IL-8 | 215.64 | 0.26 | 0.20 |
| sICAM-1 | 528.65 | 0.23 | 0.10 |

Application to various subject groups:

| | | | | | Normal |
|---|---|---|---|---|---|
| | EGDT alive | EGDT alive | | EGDT alive | therapy alive |
| | v. normal | v. normal | Alive v. | v. EGDT | v. normal |
| | therapy, alive | therapy, dead | dead, entire | dead by | therapy dead |
| | >28 d. | by 28 d. | population | 28 d. | by 28 d. |
| N (disease)/N | 31/31 | 21/16 | 62/37 | 31/16 | 31/21 |
| (nondisease) | | | | | |
| Panel response | 0.296 | 0.412 | 0.349 | 0.357 | 0.337 |
| cutoff | | | | | |
| ROC area | 0.635 | 0.723 | 0.760 | 0.947 | 0.602 |
| Std dev | 0.046 | 0.047 | 0.026 | 0.012 | 0.047 |
| Sensitivity @ | 32.6% | 18.8% | 20.8% | 93.5% | 10.1% |
| 92.5% | | | | | |
| Specificity | | | | | |
| Std dev | 7.40% | 10.50% | 8.00% | 6.10% | 8.40% |
| Sensitivity @ | 14.9% | 46.7% | 26.8% | 92.1% | 12.2% |
| 92.5% | | | | | |
| Specificity | | | | | |
| Std dev | 8.7% | 9.6% | 11.0% | 3.0% | 7.7% |

8. Optimized to separate EGDT alive >28 days vs. EGDT dead by 28 days, 8 markers

| Marker ID | Midpoint | Linear Range | Weight |
|---|---|---|---|
| MMP-9 | 1014.25 | 0.46 | 0.06 |
| IL-8 | 433.62 | 0.55 | 0.32 |
| IL-1ra | 15163.51 | 0.46 | 0.06 |
| D-Dimer | 21.45 | 0.57 | 0.17 |
| CK-BB | 3.94 | 0.51 | 0.14 |
| sFasL | 2.31 | 0.45 | 0.10 |
| CRP | 157.98 | 0.40 | 0.10 |
| sICAM-1 | 608.27 | 0.52 | 0.13 |

Application to various subject groups:

| | | | | | Normal |
|---|---|---|---|---|---|
| | EGDT alive | EGDT alive | | EGDT alive | therapy alive |
| | v. normal | v. normal | Alive v. | v. EGDT | v. normal |
| | therapy, alive | therapy, dead | dead, entire | dead by | therapy dead |
| | >28 d. | by 28 d. | population | 28 d. | by 28 d. |
| N (disease)/N | 50/45 | 29/28 | 95/57 | 50/28 | 45/29 |
| (nondisease) | | | | | |
| Panel response | 0.235 | 0.360 | 0.279 | 0.297 | 0.277 |
| cutoff | | | | | |
| ROC area | 0.590 | 0.719 | 0.719 | 0.867 | 0.574 |
| Std dev | 0.031 | 0.029 | 0.015 | 0.013 | 0.027 |
| Sensitivity @ | 20.8% | 19.9% | 17.0% | 62.5% | 7.9% |
| 92.5% | | | | | |
| Specificity | | | | | |
| Std dev | 5.50% | 8.60% | 8.30% | 10.10% | 5.90% |
| Sensitivity @ | 12.1% | 43.8% | 30.2% | 74.4% | 14.7% |
| 92.5% | | | | | |
| Specificity | | | | | |
| Std dev | 5.0% | 9.0% | 7.4% | 7.2% | 5.3% |

9. Optimized to all treatments alive >28 days vs. all treatments dead by 28 days, 8 markers

| Marker ID | Midpoint | Linear Range | Weight |
|---|---|---|---|
| BNP | 1772.08 | 0.46 | 0.10 |
| IL-8 | 752.64 | 0.58 | 0.27 |
| IL-1ra | 23192.41 | 0.39 | 0.11 |
| D-Dimer | 27.29 | 0.56 | 0.08 |
| CK-BB | 5.92 | 0.69 | 0.10 |
| sFasL | 3.13 | 0.62 | 0.13 |
| CRP | 139.34 | 0.51 | 0.10 |
| Caspase-3 | 4.73 | 0.26 | 0.18 |

Application to various subject groups:

| | | | | | Normal |
|---|---|---|---|---|---|
| | EGDT alive | EGDT alive | | EGDT alive | therapy alive |
| | v. normal | v. normal | Alive v. | v. EGDT | v. normal |
| | therapy, alive | therapy, dead | dead, entire | dead by | therapy dead |
| | >28 d. | by 28 d. | population | 28 d. | by 28 d. |
| N (disease)/N | 55/45 | 32/28 | 100/60 | 55/28 | 45/32 |
| (nondisease) | | | | | |
| Panel response | 0.131 | 0.222 | 0.159 | 0.158 | 0.164 |
| cutoff | | | | | |
| ROC area | 0.565 | 0.586 | 0.765 | 0.826 | 0.702 |
| Std dev | 0.030 | 0.042 | 0.018 | 0.027 | 0.032 |
| Sensitivity @ | 9.3% | 13.8% | 41.5% | 46.4% | 35.1% |
| 92.5% | | | | | |
| Specificity | | | | | |
| Std dev | 3.20% | 6.20% | 6.60% | 8.80% | 10.10% |
| Sensitivity @ | 9.9% | 20.1% | 45.6% | 57.9% | 22.8% |
| 92.5% | | | | | |
| Specificity | | | | | |
| Std dev | 5.6% | 7.3% | 5.7% | 9.9% | 8.8% |

As demonstrated by the foregoing tables, prognostic panels can be defined using a number of different marker combinations. Depending on the selection of “diseased” and “nondiseased” populations, the resulting panels can provide additional prognostic information, depending upon the treatment regimen. As described herein, the average ROC area provides an indication of how well the two groups under study may be discriminated using the particular panel (defined by the markers and their associated parameters). A plurality of panel response thresholds can be calculated from the same panel (or from different subsets of markers in the same panel), each threshold providing different information. For example, as SIRS, sepsis, severe sepsis, septic shock, and MODS represent different, but related, clinical states, thresholds can be established to provide mortality data for each clinical state. Alternatively, one threshold can provide prognostic information, another threshold can provide diagnostic information, and/or another threshold can provide treatment assignment.

### Example 4

### Ruling In and Out Treatment Regimens

As discussed in Rivers et al., N. Engl. J. Med. 345: 1368-77, 2001, page 1376, certain types of treatment that can be quite beneficial in certain sepsis patients can be deleterious in others. The panels of the present invention can be used to identify when such treatments might have variable risk for subjects, and to select subjects for assignment to particular treatment groups. In this regard, a panel was optimized to separate EGDT alive >28 days vs. EGDT dead by 28 days using the following markers: IL-8, D-dimer, caspase-3, sICAM-1, IL-1ra, IL-6, and CRP. A cutoff was established at the knee of the ROC curve provided by this panel. Segregating EGDT subjects and subjects receiving conventional therapy on their individual panel response values relative to the knee value provides the following data:

| | Panel response < Knee | | Panel response ≧ Knee | |
|---|---|---|---|---|
| Therapy | Conventional | EGDT | Conventional | EGDT |
| Alive (28 d) | 28 | 46 | 22 | 8 |
| Dead (28 d) | 11 | 3 | 17 | 28 |
| % Alive | 72% | 94% | 56% | 22% |

It is apparent from this table that EGDT is beneficial in those subjects having a panel response <the knee value (Mental-Haenszel Chi-square p=0.005), and deleterious in those subjects having a panel response ≧the knee value (Mental-Haenszel Chi-square p=0.0027). Improved outcomes (e.g., improved survival) should be obtainable by assigning subjects to a particular therapy (e.g., conventional or EGDT) based on the individual panel response for that subject.

In contrast, a second panel was optimized to separate conventional therapy subjects alive >28 days vs. conventional therapy subjects dead by 28 days using the same markers, and a cutoff was established at the knee of the ROC curve provided by this panel. Segregating EGDT subjects and subjects receiving conventional therapy on their individual panel response values relative to the knee value provides the following data:

| | Panel response < Knee | | Panel response ≧ Knee | |
|---|---|---|---|---|
| Therapy | Conventional | EGDT | Conventional | EGDT |
| Alive (28 d) | 46 | 49 | 4 | 5 |
| Dead (28 d) | 14 | 21 | 14 | 10 |
| % Alive | 77% | 70% | 22% | 33% |

This panel provides prognostic information on both populations, but does not provide a statistically significant basis for assigning subjects to a particular therapy.

### Example 5

### Use of Individual Markers

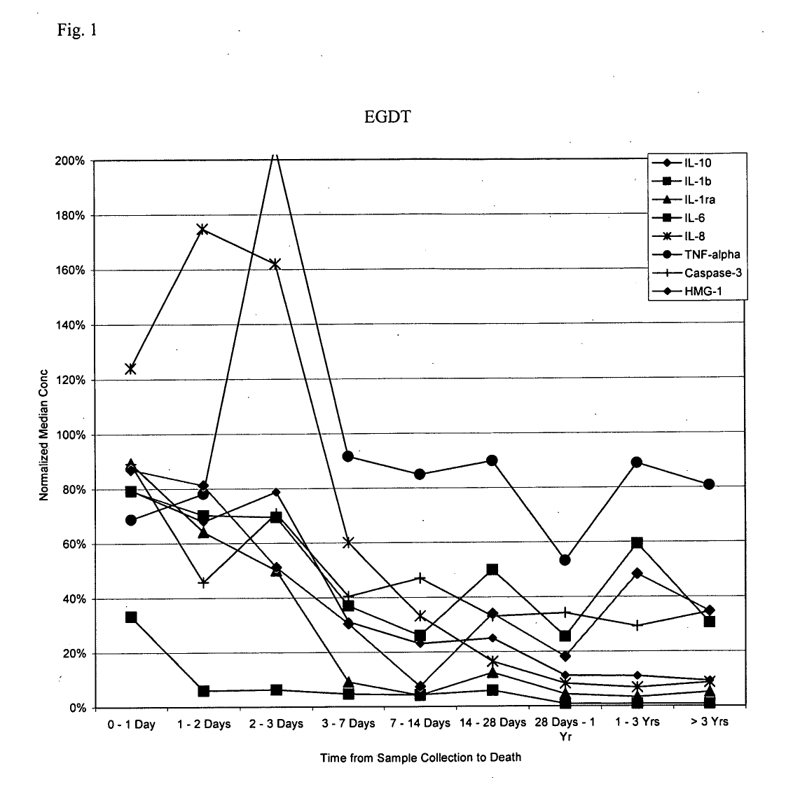

In addition to their use in panels, the various markers described herein, and particularly the markers used in the foregoing examples, may also be used individually to provide prognostic and course-of-treatment information. FIGS. 1 and 2 show levels of various markers in subjects (normalized to the median concentration of an individual marker so they may be plotted together), relative to the time of death. As can be see, these markers individyally provide prognostic data, particularly in the time 0-7, and more particularly 0-3, days before death. But predictive value may be obtained at even longer time horizons> Considering IL-6 for example, the following data was obtained:

| | n | mean IL-6 | median IL-6 | Std. Dev. |
|---|---|---|---|---|
| Death ≦28 days | | | | |
| Conventional Therapy | 41 | 3994.81 | 1194.35 | 4501.93 |
| EGDT | 37 | 3301.80 | 890.09 | 3959.09 |
| Alive >28 days | | | | |
| Conventional Therapy | 64 | 2750.15 | 398.10 | 3817.64 |
| EGDT | 73 | 1344.14 | 157.11 | 2636.39 |

For patients that survived at least 28 days, the mean IL-6 data is significantly lower (Wilcoxon test) in subject receiving EGDT as compared to conventional therapy, and also in patients that survived at least 28 days as compared to those who died in both treatment groups.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1. A method for assigning a therapy regimen and/or assigning a prognosis to a subject diagnosed with or suspected of suffering from SIRS, sepsis, severe sepsis, septic shock, or MODS, comprising:
performing an assay method on a sample obtained from said subject, wherein said assay method provides one or more detectable signals related to the presence or amount of one or more subject-derived markers independently selected from the group consisting of markers related to blood pressure regulation, markers related to inflammation, markers related to apoptosis, and markers related to coagulation and hemostasis, or markers related to said subject-derived markers, optionally further comprising one or more detectable signals related to the presence or amount of one or more subject-derived markers of tissue injury; and
correlating the signal(s) obtained from said assay method to ruling in or out a therapy regimen for said subject and/or assigning a prognosis to said subject.

2. A method according to claim 1, wherein the method rules in or out an assignment of said subject to early goal-directed therapy.

3. A method according to claim 1, wherein the correlating step comprises comparing one or more subject-derived marker concentrations to a predetermined threshold level for a particular marker of interest.

4. A method according to claim 1, wherein the correlating step comprises determining the concentration of each of a plurality of subject-derived markers, calculating a single panel response value based on the concentration of each of said plurality of subject-derived markers, and comparing the panel response value to one or more predetermined threshold levels for said panel response value.

5. A method according to claim 1, wherein the correlating step comprises comparing one or more subject-derived marker concentrations to a predetermined threshold level for a particular marker of interest and determining the concentration of each of a plurality of subject-derived markers, calculating a single panel response value based on the concentration of each of said plurality of subject-derived markers, and comparing the panel response value to a predetermined threshold level for said panel response value.

6. A method according to claim 1, wherein said one or more subject-derived markers comprise at least one marker selected from the group consisting of matrix metalloproteinase 9 (MMP-9), interleukin-1β (IL-1β), interleukin-6 (IL-6), interleukin-8 (IL-8), IL-8₆₋₇₇, interleukin-10 (IL-10), interleukin-22 (IL-22), IL-1 receptor agonist (IL-1ra), CXCL6, CXCL13, CXCL16, CCL8, CCL20, CCL23, CCL26, D-dimer, HMG-1, tumor necrosis factor-α (TNF-α), B-type natriuretic protein (BNP), A-type natriuretic protein (ANP), C-type natriuretic protein (BNP), C-reactive protein (CRP), caspase-3, calcitonin, procalcitonin₃₋₁₁₆, soluble DPP-IV, soluble FAS ligand (sFasL), creatine kinase-BB (CK-BB), vascular endothelial growth factor (VEGF), myeloperoxidase (MPO), and soluble intercellular adhesion molecule-1 (sICAM-1), or one or more markers related to said subject-derived markers.

7. A method according to claim 6, wherein said one or more subject-derived markers comprise at least one marker related to BNP selected from the group consisting of NT-proBNP, proBNP, BNP₇₉₋₁₀₈, and BNP₃₋₁₀₈.

8. A method according to claim 1, wherein the correlating step comprises determining the concentration of each of a plurality of subject-derived markers, wherein the plurality of markers comprise at least one interleukin or one or more markers related thereto.

9. A method according to claim 1, wherein the plurality of markers comprise at least one marker related to inflammation, and at least one marker related to coagulation and hemostasis or one or more markers related thereto.

10. A method according to claim 1, wherein the plurality of markers comprise at least one marker related to inflammation, and at least one marker related to blood pressure regulation, or one or more markers related thereto.

11. A method according to claim 1, wherein the plurality of markers comprise at least one marker related to blood pressure regulation, at least one marker related to inflammation, and at least one marker related to coagulation and hemostasis, or one or more markers related thereto.

12. A method according to claim 1, wherein the sample is from a human.

13. A method according to claim 1, wherein the sample is selected from the group consisting of blood, serum, urine, cerebrospinal fluid, and plasma.

14. A method according to claim 1, wherein the assay method comprises an immunoassay.

15. A method according to claim 1, wherein the assay method comprises mass spectrometry.

16. A method according to claim 1, wherein said one or more subject-derived markers comprise one or more markers related to blood pressure regulation selected from the group consisting of ANP, BNP, a marker related to BNP, CNP, urotensin II, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, renin, and urodilatin, or one or more markers related thereto.

17. A method according to claim 1, wherein said one or more subject-derived markers comprise one or more markers related to inflammation selected from the group consisting of acute phase reactants, vascular cell adhesion molecule, intercellular adhesion molecule-1, intercellular adhesion molecule-2, intercellular adhesion molecule-3, CRP, HMG-1, IL-1β, IL-6, IL-8, IL-8₆₋₇₇, IL-1ra, MCP-1; caspase-3, lipocalin-type prostaglandin D synthase, mast cell tryptase, eosinophil cationic protein, KL-6, haptoglobin, TNF-α, TNF-β, TREM-1, fibronectin, macrophage migration inhibitory factor, and VEGF, or one or more markers related thereto.

18. A method according to claim 17, wherein said one or more subject-derived markers comprise one or more acute phase reactants selected from the group consisting of hepcidin, HSP-60, HSP-65, HSP-70, sFasL, asymmetric dimethylarginine, matrix metalloproteins 11, 3, and 9, defensin HBD 1, defensin HBD 2, serum amyloid A, oxidized LDL, insulin like growth factor, TNF-β, an inter-α-inhibitor, e-selectin, glutathione-S-transferase, hypoxia-inducible factor-1α, inducible nitric oxide synthase, intracellular adhesion molecule, lactate dehydrogenase, monocyte chemoattractant peptide-1, n-acetyl aspartate, prostaglandin E2, receptor activator of nuclear factor ligand, TNF receptor superfamily member 1A, and cystatin C, or one or more markers related thereto.

19. A method according to claim 1, wherein said one or more subject-derived markers comprise one or more markers related to coagulation and hemostasis selected from the group consisting of plasmin, fibrinogen, D-dimer, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, von Willebrand factor, tissue factor, and thrombus precursor protein, or one or more markers related thereto.

20. A method according to claim 1, wherein said one or more subject-derived markers comprise one or more markers selected from the group consisting of CRP, HMG-1, caspase-3, creatine kinase-BB, MMP-9, IL-1β, IL-1ra, IL-6, IL-8, TNFα, MIF, MCP-1, BNP, CNP, pro-BNP, pro-CNP, NT-pro-BNP, tissue factor, von Willebrand factor, vWF-A1, vWF-integrin binding domain, and vWF-A3, or one or more markers related thereto.

21. A method according to claim 1, wherein said one or more subject-derived markers comprise BNP or a marker related to BNP.

22. A method according to claim 21, wherein said one or more subject-derived markers further comprise one or more markers selected from the group consisting of CRP, HMG-1, HSP-60, IL-1ra, MMP-9, an interleukin, CK-BB, sICAM-1, caspase-3, tissue factor, TNF-α, sFasL, MPO, VEGF, D-dimer, and MCP-1, or one or more markers related thereto.

23. A method according to claim 1, wherein the method rules in or out one or more treatments for inclusion in a therapy regimen selected from the group consisting of administration of intravenous antibiotic therapy, maintenance of a central venous pressure of 8-12 mm Hg, administration of crystalloids and/or colloids, maintenance of a mean arterial pressure of ≧65 mm Hg, administration of one or more vasopressors, administration of one or more vasodilators, administration of one or more corticosteroids, administration of recombinant activated protein C, maintenance of a central venous oxygen saturation of ≧70%, administration of transfused red blood cells to a hematocrit of at least 30%, administration of one or more inotropics, and administration of mechanical ventilation.
